(19) United States
(12) Patent Application Publication
(10) Pub. No.: US 2006/0241038 A1
(43) Pub. Date: Oct. 26, 2006

(54) Therapeutic agent for Abeta related disorders

(75) Inventors: Hideki Watanabe, Tsukuba-shi (JP); Francois Bernier, Tsukuba-shi (JP); Takehiko Miyagawa, Tsukuba-shi (JP)

Correspondence Address: DARBY & DARBY P.C., P. O. BOX 5257, NEW YORK, NY, 10150-5257, US

(73) Assignee: Eisai Co., Ltd., Tokyo (JP)

(21) Appl. No.: 11/111,504

(22) Filed: Apr. 20, 2005

(51) Int. Cl. A61K 38/54

(52) U.S. Cl. 514/12

(57) ABSTRACT

The present invention provides a pharmaceutical composition comprising at least one member selected from a compound capable of enhancing Aβ37 production, a compound capable of inhibiting Aβ40 and Aβ42 production and enhancing Aβ37 production, and salts of the compounds and hydrates thereof.

### FIELD OF THE INVENTION

The present invention relates to the utility of a compound capable of enhancing Aβ37 production, a compound capable of inhibiting Aβ40 and Aβ42 production and enhancing Aβ37 production, a salt thereof, a hydrate thereof or a combination thereof as a pharmaceutical composition for treating Aβ-based diseases such as Alzheimer's disease and Down's syndrome.

### BACKGROUND OF THE INVENTION

Alzheimer's disease (AD) or senile dementia of the Alzheimer's type (SDAT) is a neurodegenerative disease associated with progressive dementia symptoms. Therapeutic agents mainly used for these diseases are agents for symptom amelioration, as typified by acetylcholinesterase inhibitors. For this reason, there has been a strong social demand for the development of inhibitors of symptom progression. Some theories have been proposed for the cause of AD or SDAT, including the amyloid hypothesis focusing on abnormal accumulation of amyloid β protein (Aβ), one of the major components of senile plaques, as well as the tau theory focusing on neurofibrillary tangle formation induced by abnormal phosphorylation of tau. Aβ is a peptide composed of around 40 amino acids, which is produced by processing of amyloid precursor protein (APP) through cleavage at the β- and γ-sites with β- and γ-secretases, respectively (1). The Aβ peptide is also produced in healthy people and there are several species including Aβ37, Aβ38, Aβ39, Aβ40 and Aβ42 depending on the length of their amino acid sequence (C-terminal), with Aβ40 being known as a major species (2). Previous studies have indicated that Aβ42 is strongly hydrophobic and has a propensity to aggregate (i.e., to form a β-sheet structure) (3), and that Aβ42 accumulation occurs in the early stages of AD, SDAT or Down's syndrome and is followed by Aβ40 accumulation (4). It is also reported that all of APP, presenilin 1 (PS1) and presenilin 2 (PS2), which are found to be mutated in familial Alzheimer's disease (FAD), enhance Aβ42 production (5a, 5b). These findings suggest a strong correlation between Aβ (particularly Aβ42) and AD or SDAT onset. It is also believed that Aβ will induce tau phosphorylation and neurofibrillary tangle formation because the formation of neurofibrillary tangles is stimulated by intracerebral infusion of Aβ into tau transgenic mice (6) or in APP/tau double-transgenic mice (7).

Therapeutic agents for AD or SDAT proposed on the basis of the amyloid hypothesis include Aβ production inhibitors, Aβ aggregation inhibitors and Aβ degradation/clearance enhancers. As Aβ production inhibitors, compounds having a γ-secretase-inhibiting effect have been found previously (8a, 8b). However, in addition to APP, other proteins (e.g., Notch) are also reported as substrates of γ-secretase (9), and it is reported that existing γ-secretase inhibitors are always associated with an inhibitory effect against Notch processing. Since Notch plays an important role in cell differentiation, it is concerned that the inhibition of Notch processing may induce various side effects (10a, 10b). Also, the results obtained with genetically modified animals suggest that APP-C100 (or 99), a C-terminal fragment of APP produced by β-secretase cleavage and accumulating upon inhibition of γ-site cleavage, has cell toxicity in itself (11). Moreover, the APP intracellular domain (AICD), which is produced by γ-secretase cleavage, is being suggested to have a possibility of migrating into the nucleus and inducing some signaling event, as in the case of the Notch intracellular domain (NICD) (12); existing γ-secretase inhibitors are feared not only to cause Notch-induced side effects, but also to have a risk of developing side effects resulting from the accumulation of APP C-terminal fragments.

In 2001, some nonsteroidal anti-inflammatory drugs (NSAIDs), including ibuprofen, were reported to selectively inhibit Aβ42 production (13, 14). These compounds have a selective inhibitory effect against Aβ42 and also enhance Aβ38 production. Moreover, these compounds are found to create alienation in APP/Notch processing, suggesting a possibility of discovering γ-secretase inhibitors free from any Notch-inhibiting effect. Some NSAIDs are also reported to inhibit the formation of amyloid plaques in APP transgenic mice. However, their inhibitory activity against Aβ42 production is as low as several tens of μM to several hundreds of μM; the inhibitory effect against Aβ42 production alone is not sufficient to explain the effectiveness of these compounds in animal models (15).

(1) The profile of soluble amyloid β protein in cultured cell media. R. Wong, D. Sweeney, S. E. Gandy et al., J. Biol. Chem., 271(50), 31894-31902, 1996

(2) Highly conserved and disease-specific patterns of carboxyterminally truncated Aβ peptides 1-37/38/39 in addition to 1-40/42 in Alzheimer's disease and patients with chronic neuroinflammation. J. Wiltfang, H. Esselmann, M. Bibl et al., J. Neurochem., 81, 481-496, 2002

(3) The carboxy terminus of the beta amyloid protein is critical for the seeding of amyloid formation: implications for the pathogenesis of Alzheimer's disease. Jarrett J T, Berger E P, Lansbury P T Jr. Biochemistry, 32(18), 4693-7, 1999

(4) Visualization of Aβ42(43) and Aβ40 in senile plaques with end-specific Aβ-monoclonals: evidence that an initially deposited Aβ species is Aβ42(43). T. Iwatsubo, A. Odaka, N. Suzuki et al., Neuron, 13, 45-53, 1994

(5a) Familial Alzheimer's disease-Linked presenilin 1 variants elevate Aβ1-42/1-40 ratio in vitro and in vivo. D. R. Borchelt, G. Thinakaran, C. B. Eckman et al., Neuron, 17, 1005-1013, 1996

(5b) Mutation of the beta-amyloid precursor protein in familial Alzheimer's disease increases beta-protein production. M. Citron, T. Oltersdorf, C. Haass et al., Nature, 360, 672-674, 1992

(6) Formation of neurofibrillary tangles in P301L tau transgenic mice induced by Ab42 fibrils. J. Götx, F. Chen, J. van Dorpe et al., Science, 293, 1491-1495, 2001

(7) Enhanced Neurofibrillary degeneration in transgenic mice expressing mutant tau and APP. J. Lewis, D. W. Dickson, W. Lin et al., Science, 293, 1487-1491, 2001

(8a) Functional gamma-secretase inhibitors reduce beta-amyloid peptide levels in brain. H. F. Dovey, V. John, J. P. Anderson et al., J. Neurochem., 76, 173-181, 2001

(8b) A substrate-based difluoro ketone selectively inhibits Alzheimer's γ-secretase activity. M. S. Wolfe, M. Citron, T. S. Diehl et al. J. Med. Chem., 41, 6-9, 1998

(9) Notch and amyloid precursor protein are cleaved by similar γ-secretase(s). W. T. Kimberly, W. P. Esler, W. Ye and et al., Biochemistry, 42, 137-144, 2003

(10a) γ-secretase inhibitors repress thymocyte development. B. K. Hadland, N. R. Manley, D. Su et al. P.N.A.S., 98, 7487-7491, 2001

(10b) Chronic treatment with the γ-secretase inhibitor LY-411, 575 inhibits Aβ production and alters lymphopoiesis and intestinal cell differentiation. G. T. Wong, D. Manfra, F. M. Poulet et al., J. Biol. Chem., 279, 12876-12882, 2004

(11) Age-Dependent Neuronal and Synaptic Degeneration in Mice Transgenic for the C Terminus of the Amyloid Precursor Protein. M. L. Oster-Granite, D. L. McPhie, J. Greenan and R. L. Neve, J. Neurosci., 16(21), 6732-6741, 1996

(12) The γ-secretase-cleaved C-terminal fragment of amyloid precursor protein mediates signaling to the nucleus. Y. Gao and S. W. Pimplikar, P.N.A.S., 98, 14979-14984, 2001

(13) A subset of NSAIDs lower amyloidogenic Abeta42 independently of cyclooxygenase activity. S. Weggen, J. L. Eriksen, P. Das et al., Nature, 414, 212-216, 2001

(14) International Publication No. WO01/78721

(15) NSAIDS and enantiomers of flurbiprofen target γ-secretase and lower Aβ42 in vivo. J. L. Eriksen, S. A. Sagi, T. E. Smith, et al., J. Clin. Invest., 112, 440-449, 2003

### SUMMARY OF THE INVENTION

The object of the present invention is to provide a pharmaceutical composition based on a new concept for treating Aβ-based diseases such as Alzheimer's disease and Down's syndrome.

In view of the previous findings, the inventors of the present invention have believed that since amyloid plaques would be formed through Aβ40 accumulation surrounding Aβ42 cores, it is desirable to find a compound capable of inhibiting not only the production of Aβ42, but also the production of the major product Aβ40. In addition, Aβ37 and Aβ38 have been known for their presence, but there has been no report on their effects. Unexpectedly, the inventors of the present invention have now found, ahead of others, that Aβ37 and Aβ38 are extremely less toxic to cells than Aβ42 and that Aβ37 and Aβ38 have an inhibitory effect against Aβ42 aggregation. These findings suggest a possibility that enhanced production of Aβ37 inhibits cell damage and/or amyloid plaque formation caused by Aβ40 and Aβ42 (hereinafter also referred to as “Aβ40/42.”See below.). In view of the foregoing, the inventors of the present invention have made a hypothesis that a compound capable of enhancing Aβ37 production or a compound capable of inhibiting Aβ40/42 production and enhancing Aβ37 production is much safer and more efficient in inhibiting amyloid accumulation when compared to existing Aβ42 production inhibitors, thus enabling the provision of a novel therapeutic agent for Alzheimer's disease. Based on this hypothesis, the inventors of the present invention have made extensive and intensive efforts.

As a result, the inventors of the present invention have succeeded in finding compounds that have an effect of inhibiting Aβ40/42 production and enhancing Aβ37 production. From these results, it appears that compounds characterized by enhancing Aβ37 production, or compounds characterized by not only inhibiting Aβ40/42 production, but also enhancing production of Aβ37, which is less toxic to cells and exerting an inhibitory effect against Aβ42 aggregation, independently of their chemical structure are much safer and more efficient in inhibiting amyloid accumulation when compared to existing Aβ42 production inhibitors. Moreover, since Aβ37 and Aβ38 are extremely less toxic to cells than Aβ40/42 and have an inhibitory effect against Aβ42 aggregation, in another embodiment of the present invention, Aβ37 and Aβ38 are believed to inhibit amyloid accumulation. Accordingly, the inventors of the present invention have clarified that these compounds as well as Aβ37 and Aβ38 effectively serve as active ingredients of therapeutic agents based on a new concept for treating Aβ-based diseases such as Alzheimer's disease and Down's syndrome, and have completed the present invention.

Namely, the present invention is as follows.

(1) A method for inhibiting Aβ40 and Aβ42 production, which comprises using at least one member selected from the group consisting of a compound capable of enhancing Aβ37 production in the living body or a part thereof, and a salt of the compound and hydrates thereof to enhance Aβ37 production.

(2) A method for inhibiting Aβ40 and Aβ42 production and enhancing Aβ37 production, which comprises using at least one member selected from the group consisting of a compound capable of inhibiting Aβ40 and Aβ42 production and enhancing Aβ37 production in the living body or a part thereof, and a salt of the compound and hydrates thereof.

(3) A method for inhibiting Aβ aggregation, which comprises allowing Aβ37 and/or Aβ38 to act on Aβ42 in the living body or a part thereof.

(4) A method for inhibiting Aβ aggregation, which comprises using at least one member selected from the group consisting of a compound capable of enhancing Aβ37 production in the living body or a part thereof, and a salt of the compound and hydrates thereof to enhance Aβ37 production.

(5) A method for inhibiting Aβ aggregation, which comprises using at least one member selected from the group consisting of a compound capable of inhibiting Aβ40 and Aβ42 production and enhancing Aβ37 production in the living body or a part thereof, and a salt of the compound and hydrates thereof.

(6) A method for preventing nerve cell death, which comprises allowing Aβ37 and/or Aβ38 to act on Aβ42 in the living body or a part thereof.

(7) A method for preventing nerve cell death, which comprises using at least one member selected from the group consisting of a compound capable of enhancing Aβ37 production in the living body or a part thereof, and a salt of the compound and hydrates thereof to enhance Aβ37 production.

(8) A method for preventing nerve cell death, which comprises using at least one member selected from the group consisting of a compound capable of inhibiting Aβ40 and Aβ42 production and enhancing Aβ37 production in the living body or a part thereof, and a salt of the compound and hydrates thereof.

(9) The method according to any one of (1) to (8) above, wherein the part of the living body is the brain.

(10) An Aβ aggregation inhibitor which comprises at least one member selected from the group consisting of a compound capable of enhancing Aβ37 production, a compound capable of inhibiting Aβ40 and Aβ42 production and enhancing Aβ37 production, and salts of the compounds and hydrates thereof.

(11) A nerve cell death inhibitor which comprises at least one member selected from the group consisting of a compound capable of enhancing Aβ37 production, a compound capable of inhibiting Aβ40 and Aβ42 production and enhancing Aβ37 production, and salts of the compounds and hydrates thereof.

(12) A pharmaceutical composition which comprises at least one member selected from the group consisting of a compound capable of enhancing Aβ37 production, a compound capable of inhibiting Aβ40 and Aβ42 production and enhancing Aβ37 production, and salts of the compounds and hydrates thereof.

(13) The pharmaceutical composition according to (12) above, which is used for treating an Aβ-based disease.

(14) The pharmaceutical composition according to (13) above, wherein the Aβ-based disease is any one selected from the group consisting of Alzheimer's disease, senile dementia of the Alzheimer's type, mild cognitive impairment, senile dementia, Down's syndrome and amyloidosis.

(15) An Aβ aggregation inhibitor which comprises at least one member selected from the group consisting of the following peptides (a) and (b), and fragments thereof:
(a) a peptide which contains the amino acid sequence shown in any one of SEQ ID NO: 12, SEQ ID NO: 14, SEQ ID NO: 16, SEQ ID NO: 18, SEQ ID NO: 20 and SEQ ID NO: 22; and
(b) a peptide which contains an amino acid sequence derived from the amino acid sequence shown in any one of SEQ ID NO: 12, SEQ ID NO: 14, SEQ ID NO: 16, SEQ ID NO: 18, SEQ ID NO: 20 and SEQ ID NO: 22 by deletion, substitution or addition, or a combination thereof, of one or several amino acids and which has an inhibitory activity against Aβ aggregation.

(16) A nerve cell death inhibitor which comprises at least one member selected from the group consisting of the following peptides (a) and (b), and fragments thereof:
(a) a peptide which contains the amino acid sequence shown in any one of SEQ ID NO: 12, SEQ ID NO: 14, SEQ ID NO: 16, SEQ ID NO: 18, SEQ ID NO: 20 and SEQ ID NO: 22; and
(b) a peptide which contains an amino acid sequence derived from the amino acid sequence shown in any one of SEQ ID NO: 12, SEQ ID NO: 14, SEQ ID NO: 16, SEQ ID NO: 18, SEQ ID NO: 20 and SEQ ID NO: 22 by deletion, substitution or addition, or a combination thereof, of one or several amino acids and which has an inhibitory activity against Aβ aggregation.

(17) A pharmaceutical composition which comprises at least one member selected from the group consisting of the following peptides (a) and (b), and fragments thereof:
(a) a peptide which contains the amino acid sequence shown in any one of SEQ ID NO: 12, SEQ ID NO: 14, SEQ ID NO: 16, SEQ ID NO: 18, SEQ ID NO: 20 and SEQ ID NO: 22; and
(b) a peptide which contains an amino acid sequence derived from the amino acid sequence shown in any one of SEQ ID NO: 12, SEQ ID NO: 14, SEQ ID NO: 16, SEQ ID NO: 18, SEQ ID NO: 20 and SEQ ID NO: 22 by deletion, substitution or addition, or a combination thereof, of one or several amino acids and which has an inhibitory activity against Aβ aggregation.

(18) The pharmaceutical composition according to (17) above, which is used for treating an Aβ-based disease.

(19) The pharmaceutical composition according to (18) above, wherein the Aβ-based disease is any one selected from the group consisting of Alzheimer's disease, senile dementia of the Alzheimer's type, mild cognitive impairment, senile dementia, Down's syndrome and amyloidosis.

(20) An Aβ aggregation inhibitor which comprises a polynucleotide encoding at least one member selected from the group consisting of the following peptides (a) and (b), and fragments thereof:
(a) a peptide which contains the amino acid sequence shown in any one of SEQ ID NO: 12, SEQ ID NO: 14, SEQ ID NO: 16, SEQ ID NO: 18, SEQ ID NO: 20 and SEQ ID NO: 22; and
(b) a peptide which contains an amino acid sequence derived from the amino acid sequence shown in any one of SEQ ID NO: 12, SEQ ID NO: 14, SEQ ID NO: 16, SEQ ID NO: 18, SEQ ID NO: 20 and SEQ ID NO: 22 by deletion, substitution or addition, or a combination thereof, of one or several amino acids and which has an inhibitory activity against Aβ aggregation.

(21) An Aβ aggregation inhibitor which comprises at least one member selected from the group consisting of the following polynucleotides (a) and (b):
(a) a polynucleotide which contains the nucleotide sequence shown in any one of SEQ ID NO: 11, SEQ ID NO: 13, SEQ ID NO: 15, SEQ ID NO: 17, SEQ ID NO: 19 and SEQ ID NO: 21; and
(b) a polynucleotide which hybridizes, under stringent conditions, to a polynucleotide consisting of a nucleotide sequence complementary to a polynucleotide consisting of the nucleotide sequence shown in any one of SEQ ID NO: 11, SEQ ID NO: 13, SEQ ID NO: 15, SEQ ID NO: 17, SEQ ID NO: 19 and SEQ ID NO: 21 and which encodes a peptide having an inhibitory activity against Aβ aggregation.

(22) A nerve cell death inhibitor which comprises a polynucleotide encoding at least one member selected from the group consisting of the following peptides (a) and (b), and fragments thereof:
(a) a peptide which contains the amino acid sequence shown in any one of SEQ ID NO: 12, SEQ ID NO: 14, SEQ ID NO: 16, SEQ ID NO: 18, SEQ ID NO: 20 and SEQ ID NO: 22; and
(b) a peptide which contains an amino acid sequence derived from the amino acid sequence shown in any one of SEQ ID NO: 12, SEQ ID NO: 14, SEQ ID NO: 16, SEQ ID NO: 18, SEQ ID NO: 20 and SEQ ID NO: 22 by deletion, substitution or addition, or a combination thereof, of one or several amino acids and which has an inhibitory activity against Aβ aggregation.

(23) A nerve cell death inhibitor which comprises at least one member selected from the group consisting of the following polynucleotides (a) and (b):
(a) a polynucleotide which contains the nucleotide sequence shown in any one of SEQ ID NO: 11, SEQ ID NO: 13, SEQ ID NO: 15, SEQ ID NO: 17, SEQ ID NO: 19 and SEQ ID NO: 21; and
(b) a polynucleotide which hybridizes, under stringent conditions, to a polynucleotide consisting of a nucleotide sequence complementary to a polynucleotide consisting of the nucleotide sequence shown in any one of SEQ ID NO: 11, SEQ ID NO: 13, SEQ ID NO: 15, SEQ ID NO: 17, SEQ ID NO: 19 and SEQ ID NO: 21 and which encodes a peptide having an inhibitory activity against Aβ aggregation.

(24) A pharmaceutical composition which comprises a polynucleotide encoding at least one member selected from the group consisting of the following peptides (a) and (b), and fragments thereof:
(a) a peptide which contains the amino acid sequence shown in any one of SEQ ID NO: 12, SEQ ID NO: 14, SEQ ID NO: 16, SEQ ID NO: 18, SEQ ID NO: 20 and SEQ ID NO: 22; and
(b) a peptide which contains an amino acid sequence derived from the amino acid sequence shown in any one of SEQ ID NO: 12, SEQ ID NO: 14, SEQ ID NO: 16, SEQ ID NO: 18, SEQ ID NO: 20 and SEQ ID NO: 22 by deletion, substitution or addition, or a combination thereof, of one or several amino acids and which has an inhibitory activity against Aβ aggregation.

(25) A pharmaceutical composition which comprises at least one member selected from the group consisting of the following polynucleotides (a) and (b):
(a) a polynucleotide which contains the nucleotide sequence shown in any one of SEQ ID NO: 11, SEQ ID NO: 13, SEQ ID NO: 15, SEQ ID NO: 17, SEQ ID NO: 19 and SEQ ID NO: 21; and
(b) a polynucleotide which hybridizes, under stringent conditions, to a polynucleotide consisting of a nucleotide sequence complementary to a polynucleotide consisting of the nucleotide sequence shown in any one of SEQ ID NO: 11, SEQ ID NO: 13, SEQ ID NO: 15, SEQ ID NO: 17, SEQ ID NO: 19 and SEQ ID NO: 21 and which encodes a peptide having an inhibitory activity against Aβ aggregation.

(26) The pharmaceutical composition according to (24) or (25) above, which is used for treating an Aβ-based disease.

(27) The pharmaceutical composition according to (26) above, wherein the Aβ-based disease is any one selected from the group consisting of Alzheimer's disease, senile dementia of the Alzheimer's type, mild cognitive impairment, senile dementia, Down's syndrome and amyloidosis.

(28) A method for treating an Aβ-based disease, which comprises administering to a mammal in need of treatment of the disease, an effective amount of at least one member selected from the group consisting of a compound capable of enhancing Aβ37 production, a compound capable of inhibiting Aβ40 and Aβ42 production and enhancing Aβ37 production, and salts of the compounds and hydrates thereof.

(29) A method for treating an Aβ-based disease, which comprises administering to a mammal in need of treatment of the disease, an effective amount of the pharmaceutical composition according to at least one selected from the group consisting of (12), (13), (14), (17), (18), (19), (24), (25), (26) and (27) above.

(30) The method according to (28) or (29) above, wherein the Aβ-based disease is any one selected from the group consisting of Alzheimer's disease, senile dementia of the Alzheimer's type, mild cognitive impairment, senile dementia, Down's syndrome and amyloidosis.

(31) The method according to (28) or (29) above, wherein the mammal is a human.

(32) A method for identifying a compound capable of enhancing Aβ37 production, which comprises:
(a) contacting a candidate compound with a biological composition;
(b) measuring the amount of Aβ37 in the biological composition contacted with the candidate compound and the amount of Aβ37 in a biological composition not contacted with the candidate compound;
(c) selecting a candidate compound that produces an increase in the amount of Aβ37 in the biological composition contacted with the candidate compound when compared to the amount of Aβ37 in the biological composition not contacted with the candidate compound; and
(d) identifying the candidate compound obtained in (c) above as a compound capable of enhancing Aβ37 production.

(33) A method for identifying a compound capable of inhibiting Aβ40 and Aβ42 production and enhancing Aβ37 production, which comprises:
(a) contacting a candidate compound with a biological composition;
(b) measuring the amounts of Aβ40, Aβ42 and Aβ37 in the biological composition contacted with the candidate compound and the amounts of Aβ40, Aβ42 and Aβ37 in a biological composition not contacted with the candidate compound;
(c) selecting a candidate compound that causes reductions in the amounts of Aβ40 and Aβ42 and also produces an increase in the amount of Aβ37 in the biological composition contacted with the candidate compound when compared to the amounts of Aβ40, Aβ42 and Aβ37 in the biological composition not contacted with the candidate compound; and
(d) identifying the candidate compound obtained in (c) above as a compound capable of inhibiting Aβ40 and Aβ42 production and enhancing Aβ37 production.

(34) A method for screening a compound capable of enhancing Aβ37 production, which comprises:
(a) contacting a candidate compound with a biological composition;
(b) measuring the amount of Aβ37 in the biological composition contacted with the candidate compound and the amount of Aβ37 in a biological composition not contacted with the candidate compound;
(c) selecting a candidate compound that produces an increase in the amount of Aβ37 in the biological composition contacted with the candidate compound when compared to the amount of Aβ37 in the biological composition not contacted with the candidate compound; and
(d) identifying the candidate compound obtained in (c) above as a compound capable of enhancing Aβ37 production.

(35) A method for screening a compound capable of inhibiting Aβ40 and Aβ42 production and enhancing Aβ37 production, which comprises:
(a) contacting a candidate compound with a biological composition;
(b) measuring the amounts of Aβ40, Aβ42 and Aβ37 in the biological composition contacted with the candidate compound and the amounts of Aβ40, Aβ42 and Aβ37 in a biological composition not contacted with the candidate compound;
(c) selecting a candidate compound that causes reductions in the amounts of Aβ40 and Aβ42 and also produces an increase in the amount of Aβ37 in the biological composition contacted with the candidate compound when compared to the amounts of Aβ40, Aβ42 and Aβ37 in the biological composition not contacted with the candidate compound; and
(d) identifying the candidate compound obtained in (c) above as a compound capable of inhibiting Aβ40 and Aβ42 production and enhancing Aβ37 production.

(36) The method according to any one of (32) to (35) above, wherein the biological composition comprises β-amyloid precursor protein-expressing cells.

(37) The method according to any one of (32) to (35) above, wherein the biological composition comprises mammalian cells.

(38) The method according to any one of (32) to (35) above, wherein the biological composition comprises nerve cells.

(39) A pharmaceutical composition which comprises at least one member selected from the group consisting of a compound capable of enhancing Aβ37 production, a compound capable of inhibiting Aβ40 and Aβ42 production and enhancing Aβ37 production, and salts of the compounds and hydrates thereof, as well as at least one member selected from the group consisting of a cholinesterase-inhibiting substance, an NMDA receptor antagonist and an AMPA receptor antagonist.

(40) The pharmaceutical composition according to (39) above, wherein the cholinesterase-inhibiting substance is donepezil or a salt thereof.

(41) The pharmaceutical composition according to (39) above, wherein the NMDA receptor antagonist is memantine.

(42) The pharmaceutical composition according to (39) above, wherein the AMPA receptor antagonist is talampanel.

(43) The pharmaceutical composition according to any one of (39) to (42) above, which is a therapeutic agent for an Aβ-based disease.

(44) The pharmaceutical composition according to (43) above, wherein the Aβ-based disease is any one selected from the group consisting of Alzheimer's disease, senile dementia of the Alzheimer's type, mild cognitive impairment, senile dementia, Down's syndrome and amyloidosis.

(45) A method for treating an Aβ-based disease, which comprises administering to a mammal in need of treatment of the disease, an effective amount of at least one member selected from the group consisting of a compound capable of enhancing Aβ37 production, a compound capable of inhibiting Aβ40 and Aβ42 production and enhancing Aβ37 production, and salts of the compounds and hydrates thereof, as well as an effective amount of at least one member selected from the group consisting of a cholinesterase-inhibiting substance, an NMDA receptor antagonist and an AMPA receptor antagonist.

(46) The method according to (45) above, wherein the cholinesterase-inhibiting substance is donepezil or a salt thereof.

(47) The method according to (45) above, wherein the NMDA receptor antagonist is memantine.

(48) The method according to (45) above, wherein the AMPA receptor antagonist is talampanel.

(49) The method according to any one of (45) to (48) above, which is a therapeutic agent for an Aβ-based disease.

(50) The method according to (49) above, wherein the Aβ-based disease is any one selected from the group consisting of Alzheimer's disease, senile dementia of the Alzheimer's type, mild cognitive impairment, senile dementia, Down's syndrome and amyloidosis.

(51) The method according to any one of (45) to (50) above, wherein the mammal is a human.

(52) A kit which comprises at least one member selected from the group consisting of a compound capable of enhancing Aβ37 production, a compound capable of inhibiting Aβ40 and Aβ42 production and enhancing Aβ37 production, and salts of the compounds and hydrates thereof, as well as at least one member selected from the group consisting of a cholinesterase-inhibiting substance, an NMDA receptor antagonist and an AMPA receptor antagonist.

(53) The kit according to (52) above, wherein the cholinesterase-inhibiting substance is donepezil or a salt thereof.

(54) The kit according to (52) above, wherein the NMDA receptor antagonist is memantine.

(55) The kit according to (52) above, wherein the AMPA receptor antagonist is talampanel.

(56) The inhibitor according to (15) above, wherein the peptides (a) and (b) and fragments thereof are in the form of a salt or a hydrate thereof.

(57) The inhibitor according to (16) above, wherein the peptides (a) and (b) and fragments thereof are in the form of a salt or a hydrate thereof.

(58) The pharmaceutical composition according to (17) above, wherein the peptides (a) and (b) and fragments thereof are in the form of a salt or a hydrate thereof.

(59) The inhibitor according to (20) or (21) above, wherein the polynucleotide(s) is/are in the form of a salt or a hydrate thereof.

(60) The inhibitor according to (22) or (23) above, wherein the polynucleotide(s) is/are in the form of a salt or a hydrate thereof.

(61) The pharmaceutical composition according to (24) or (25) above, wherein the polynucleotide(s) is/are in the form of a salt or a hydrate thereof.

### BRIEF DESCRIPTION OF THE DRAWINGS

FIG. 1: Results of circular dichroism (CD) measurement for Aβ1-37, Aβ1-38, Aβ1-40 and Aβ1-42 (10 μM each)

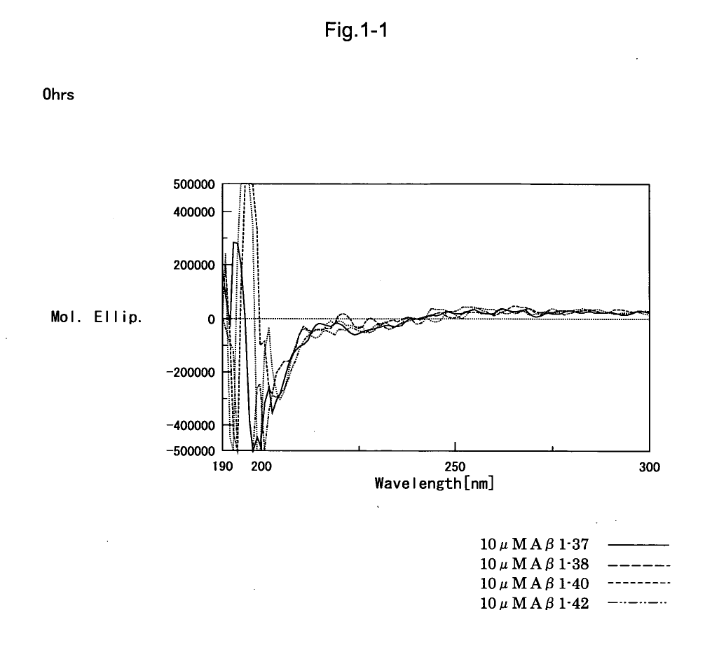

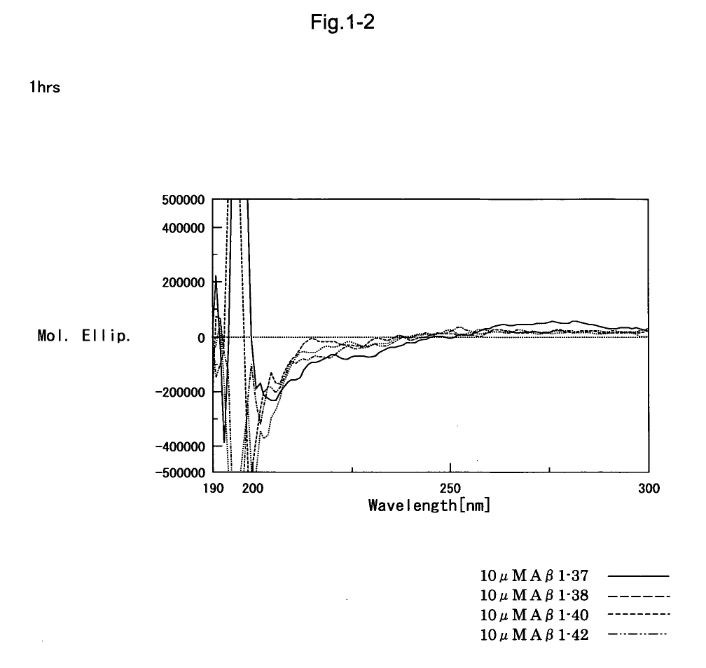

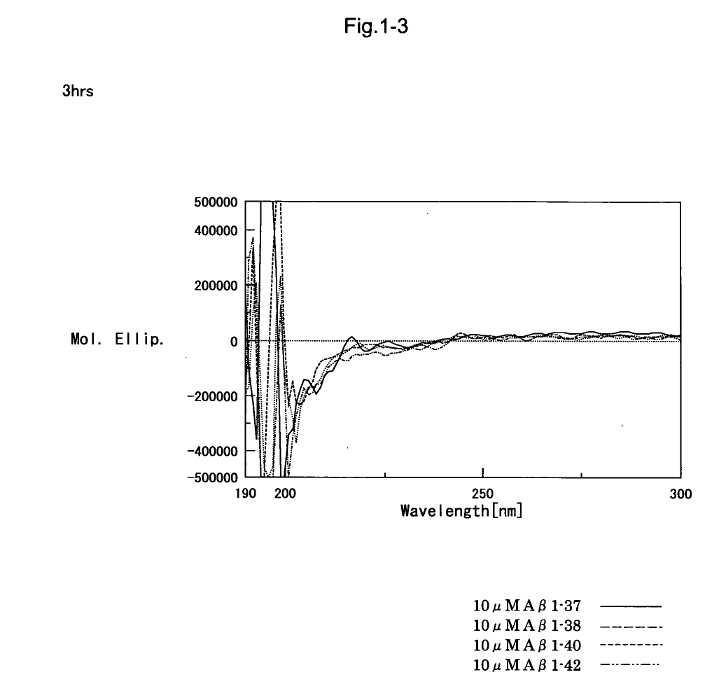

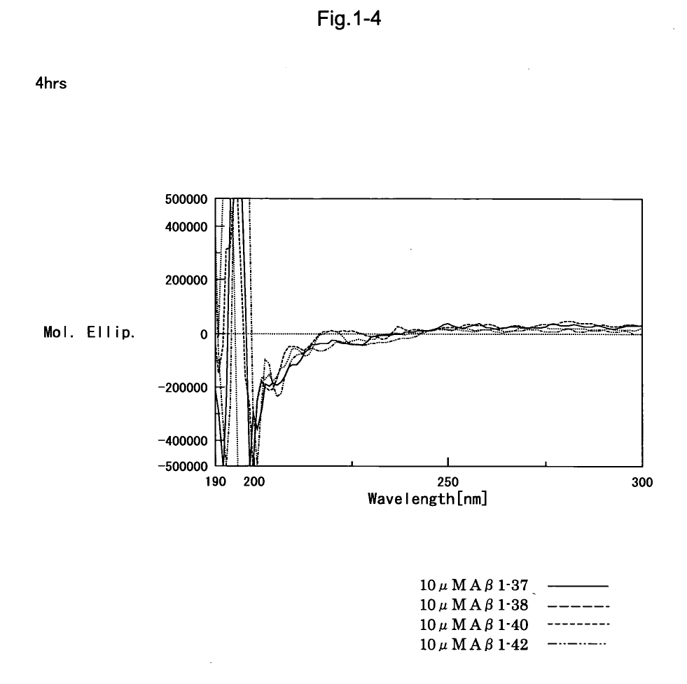

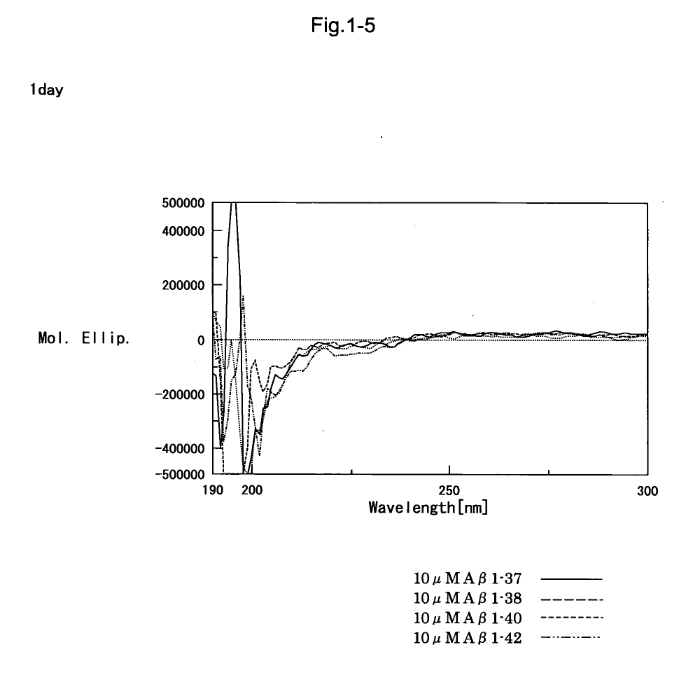

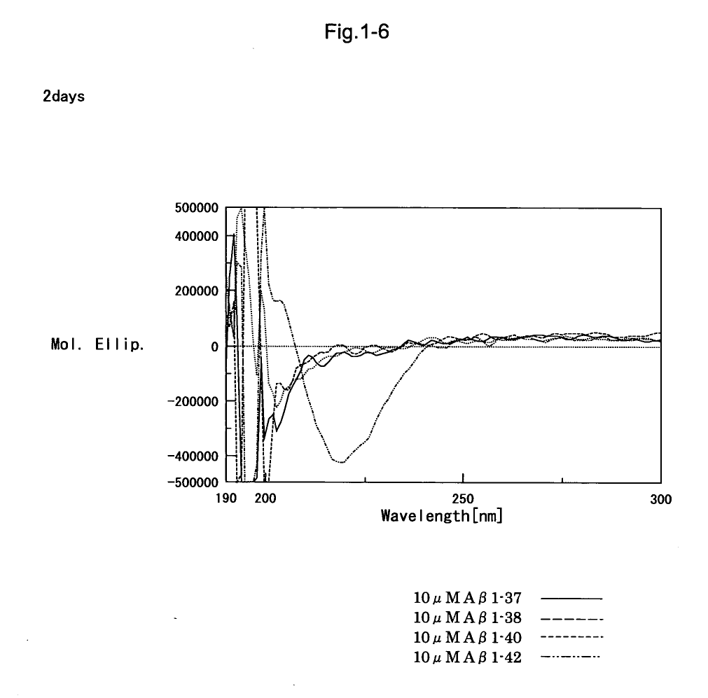

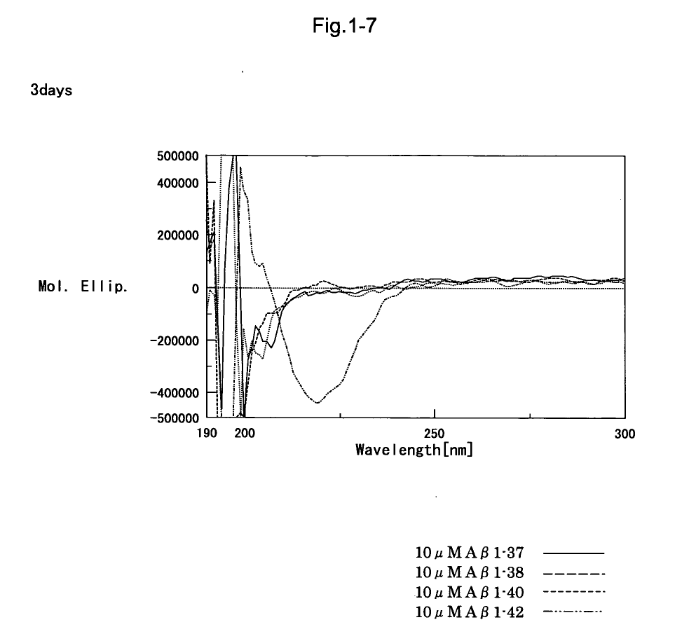

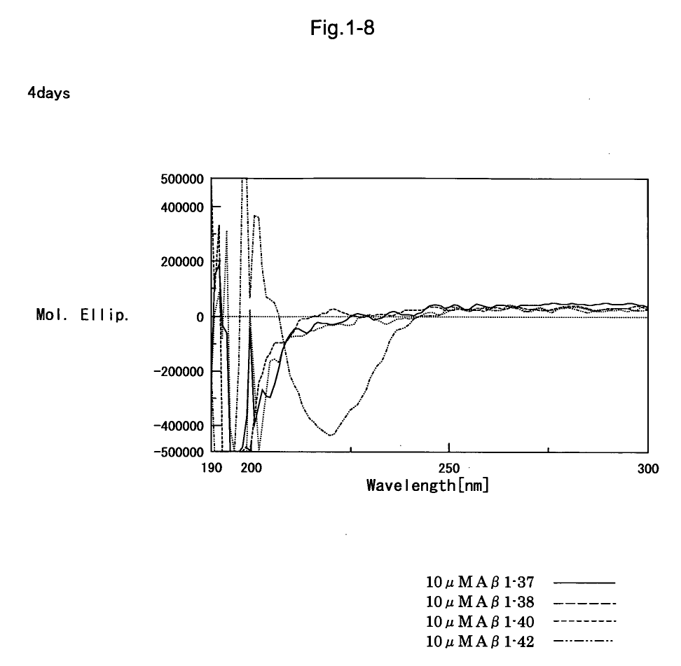

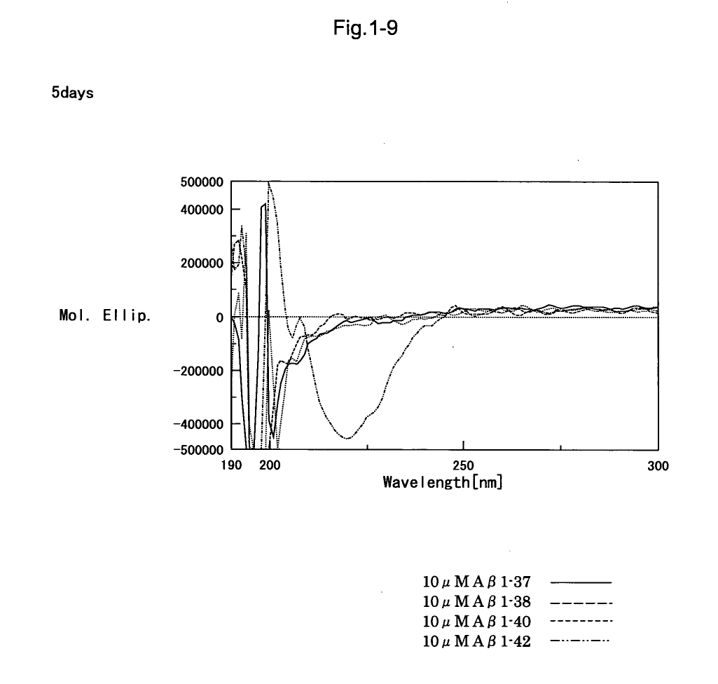

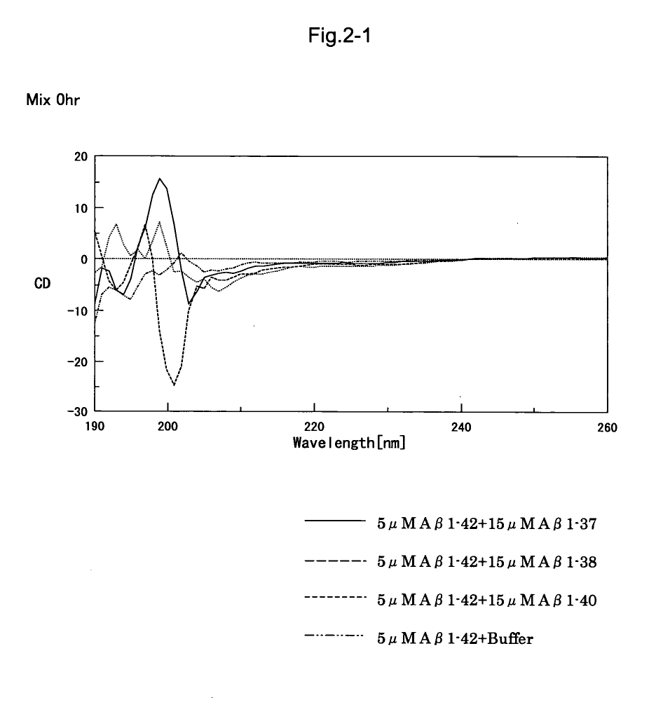

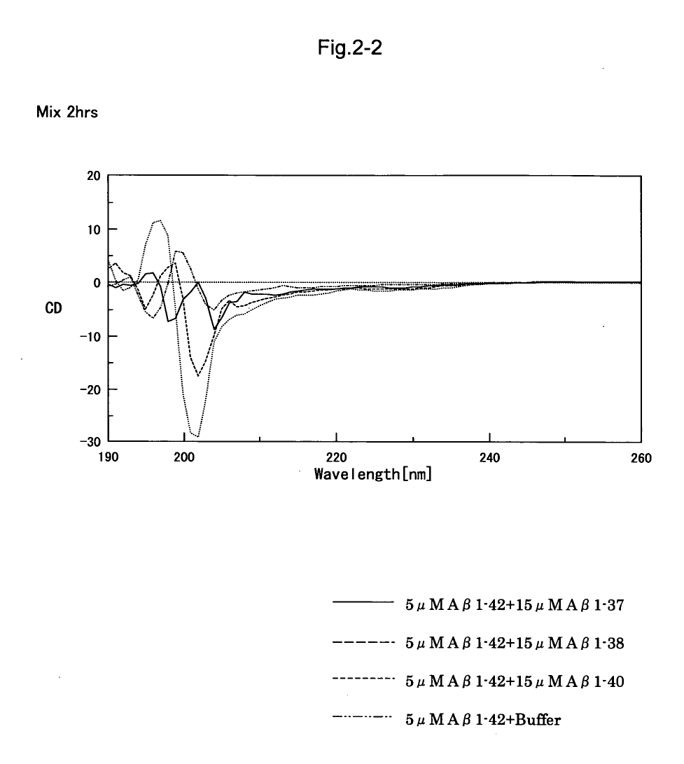

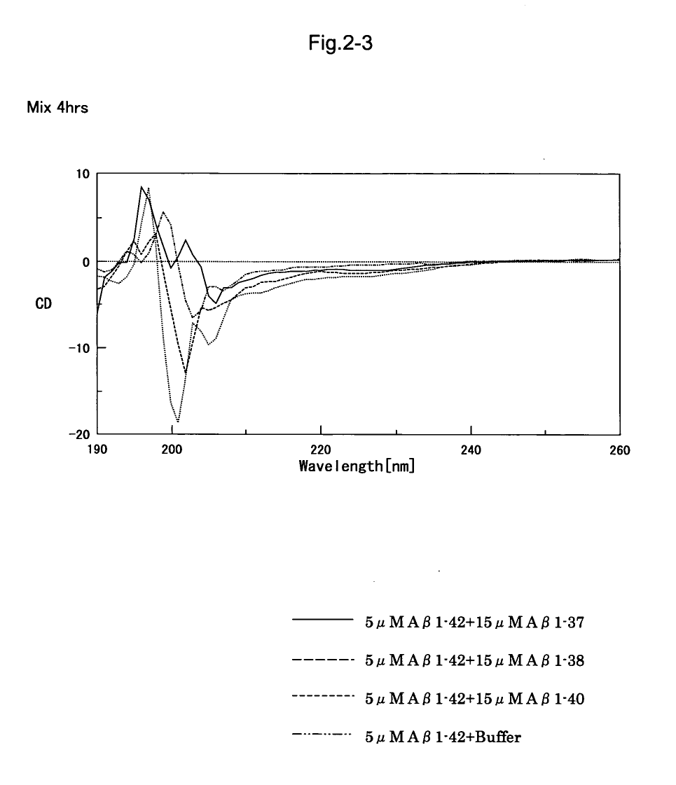

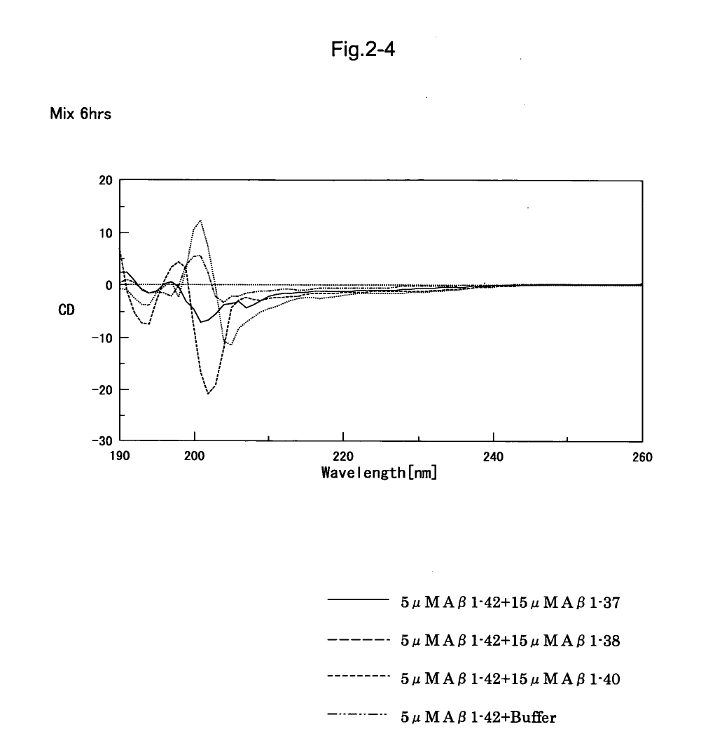

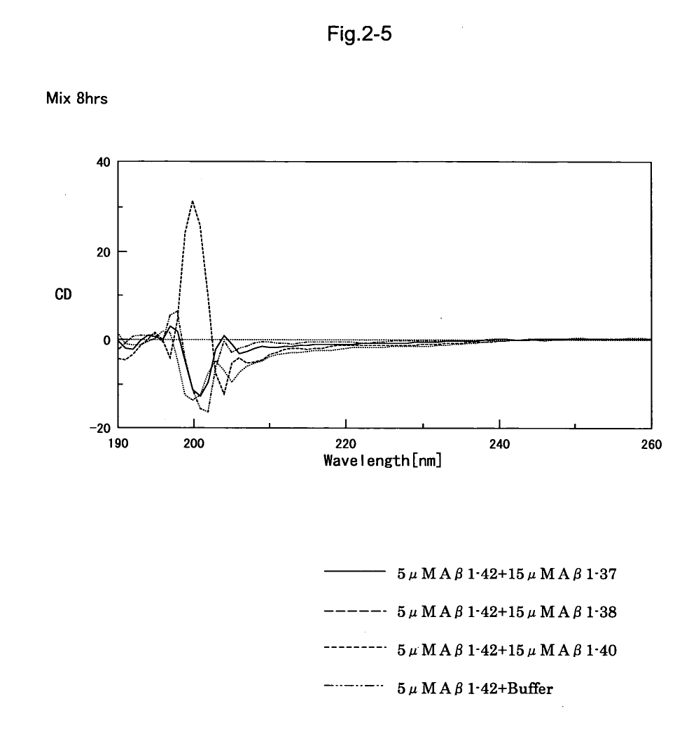

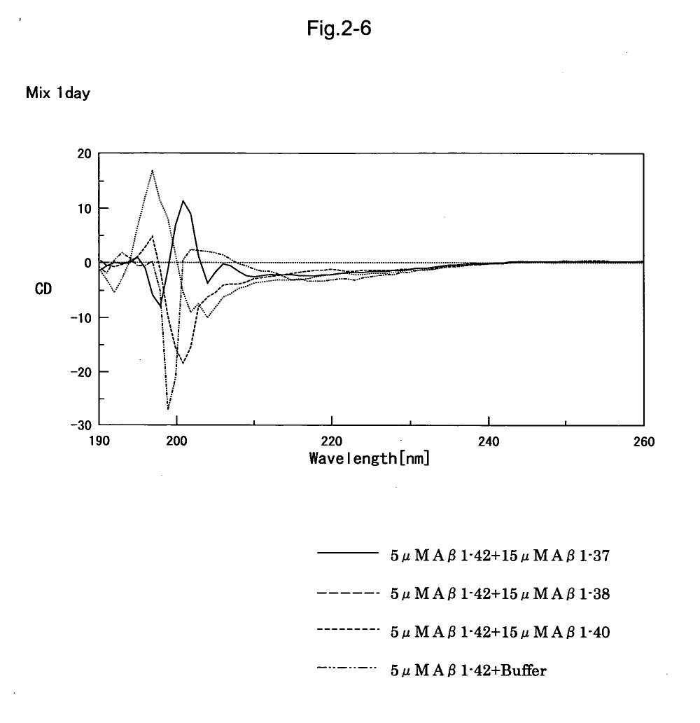

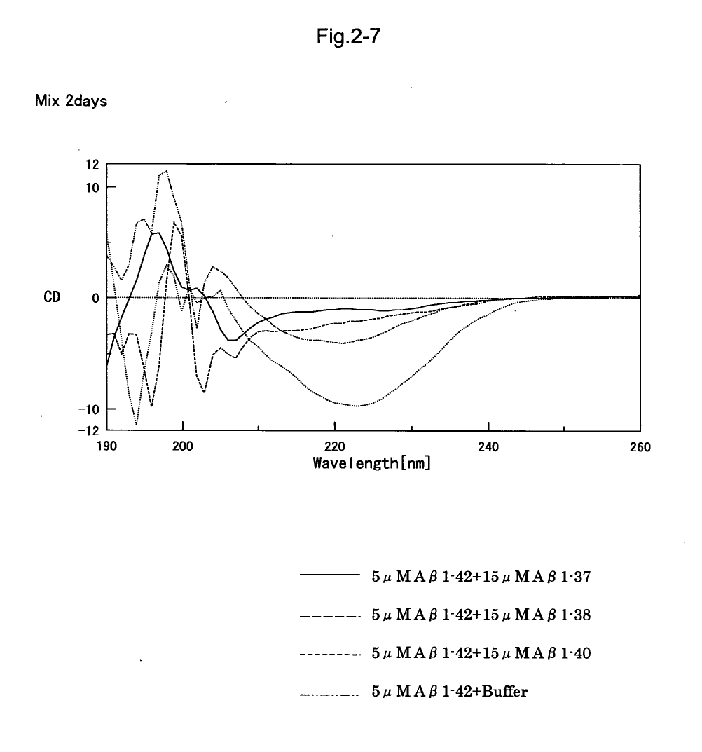

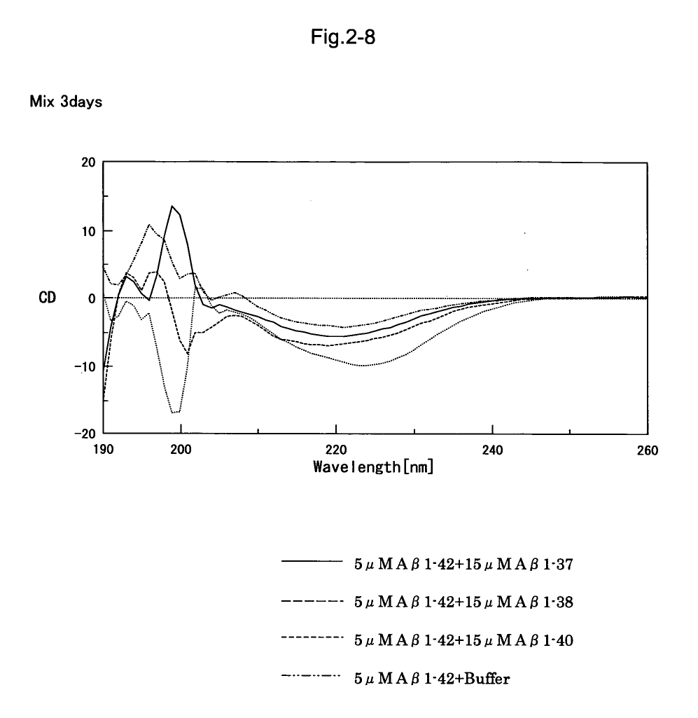

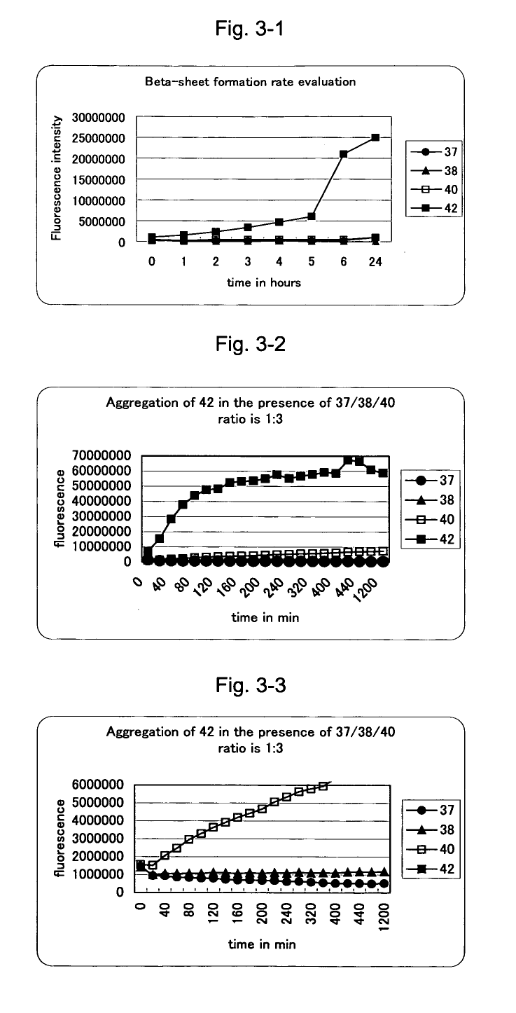

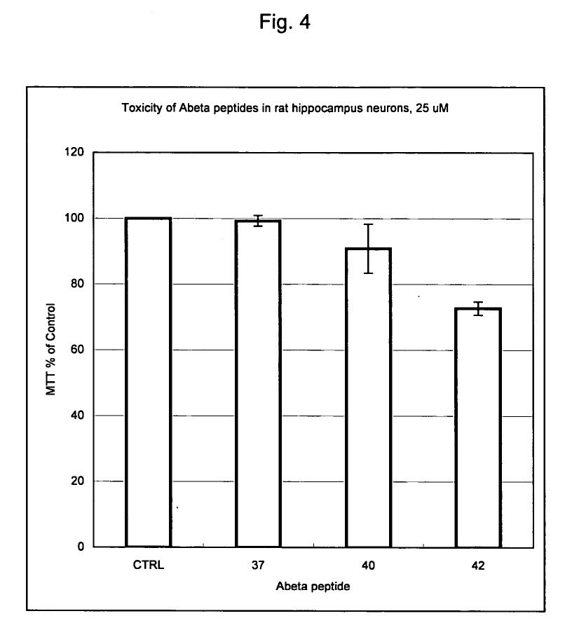

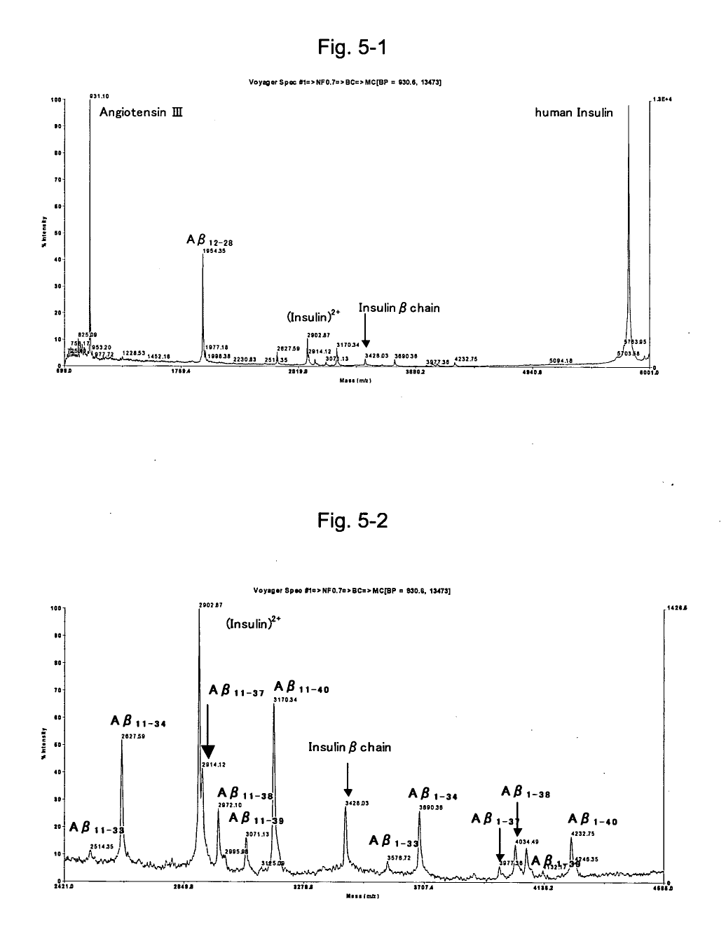

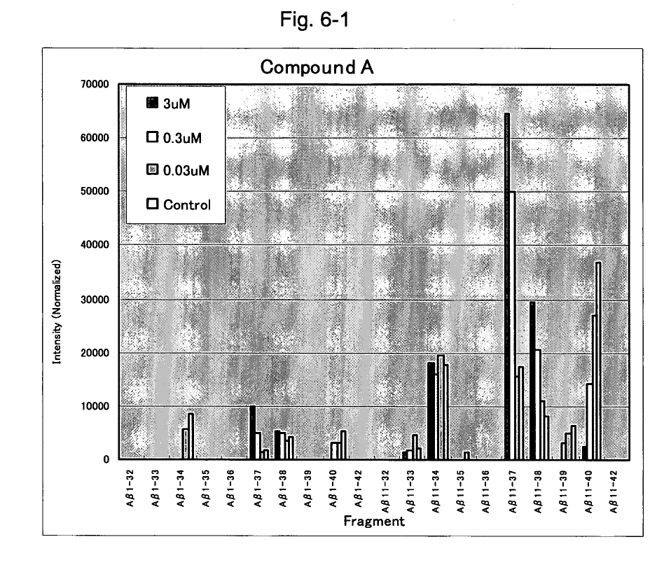

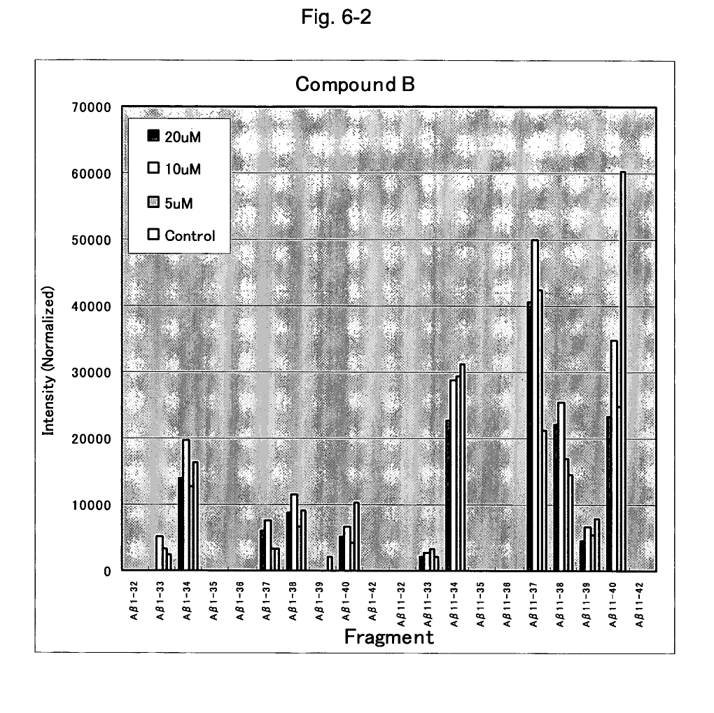

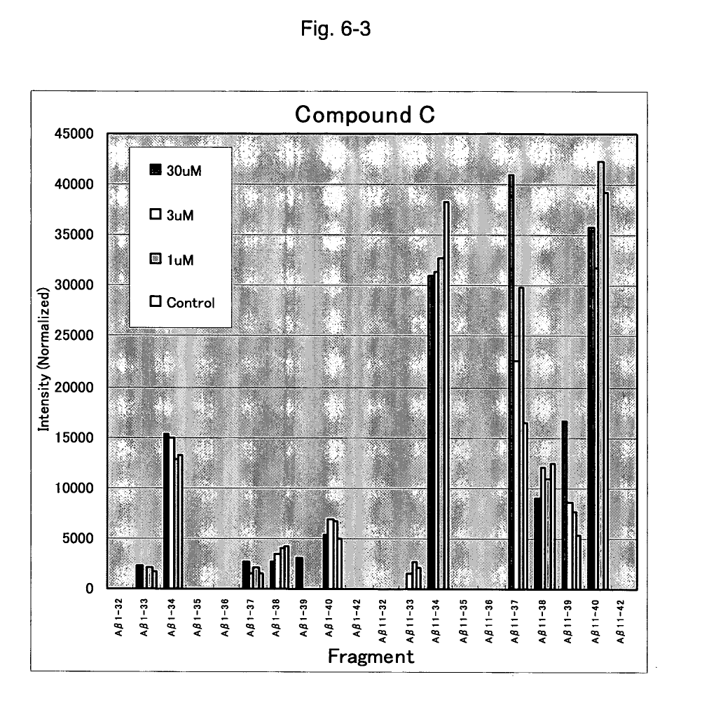

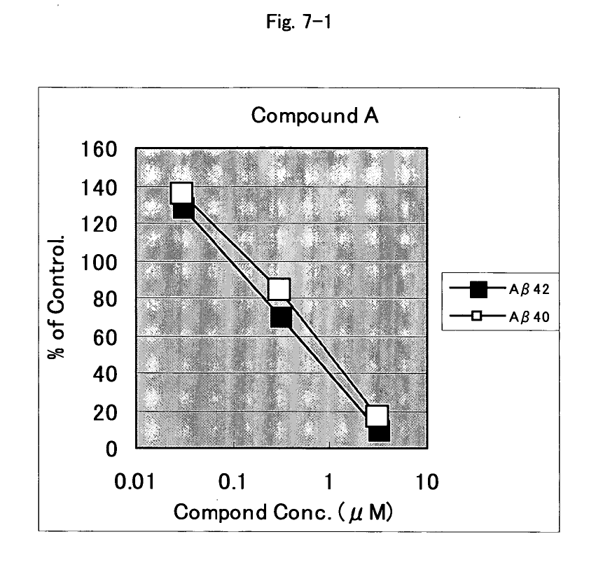

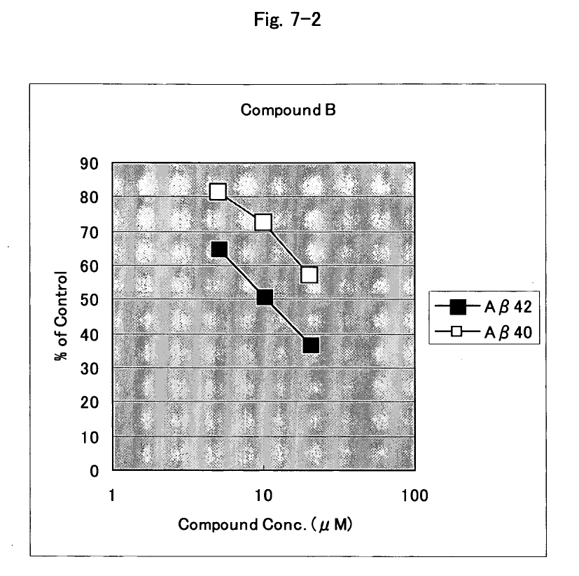

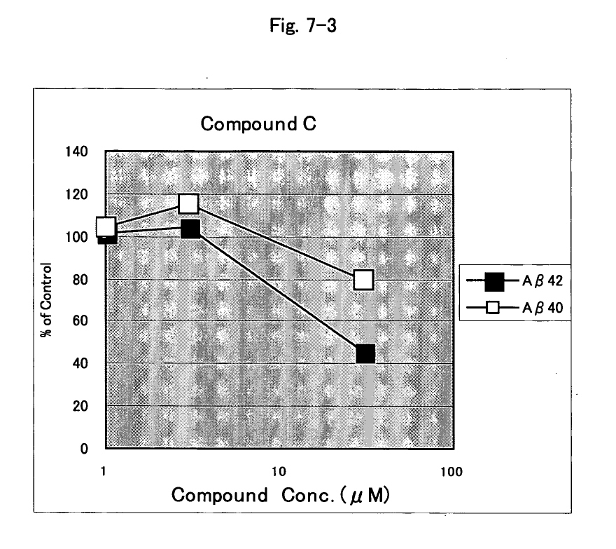

The vertical axis represents the degree of circular polarization and the horizontal axis represents the wavelength for measurement. CD spectra were obtained for each Aβ sample immediately after dissolving in a solution of 10 mM HEPES containing 0.9% NaCl (FIG. 1-1) and after 1 hour (FIG. 1-2), after 3 hours (FIG. 1-3), after 4 hours (FIG. 1-4), after 1 day (FIG. 1-5), after 2 days (FIG. 1-6), after 3 days (FIG. 1-7), after 4 days (FIG. 1-8) and after 5 days (FIG. 1-9). The waveform with a minimum around 220 nm wavelength indicates a β-sheet structure. At 1 day after dissolution, none of the Aβ samples was in a β-sheet structure (FIG. 1-5). At 2 days after dissolution, only Aβ1-42 showed a waveform characteristic of β-sheet structure (FIG. 1-6) and remained stable until 5 days after dissolution (FIG. 1-9). Aβ1-37, Aβ1-38 and Aβ1-40 showed no β-sheet structure formation even at 5 days after dissolution (FIG. 1-9).

FIG. 2: Results of CD measurement for Aβ1-42 when mixed with Aβ1-37, Aβ1-38 or Aβ1-40

CD spectra were obtained for 5 μM Aβ1-42 immediately after mixing with 15 μM Aβ1-37, Aβ1-38 or Aβ1-40 (FIG. 2-1) and after 2 hours (FIG. 2-2), after 4 hours (FIG. 2-3), after 6 hours (FIG. 2-4), after 8 hours (FIG. 2-5), after 1 day (FIG. 2-6), after 2 days (FIG. 2-7) and after 3 days (FIG. 2-8). Until 8 hours after mixing, all the Aβ samples were believed to have random structures (FIG. 2-5). From 1 day after dissolution, only Aβ1-42+buffer showed a β-sheet structure (FIG. 2-6). In the sample mixed with Aβ1-40, a CD spectrum indicative of a β-sheet structure was detected after 2 days (FIG. 2-7). In the sample mixed with Aβ1-37 or Aβ1-38, a CD spectrum indicative of a β-sheet structure was detected after 3 days (FIG. 2-8). In particular, it was suggested that Aβ1-37 and Aβ1-38 may have a strong effect of delaying β-sheet structure formation in Aβ1-42 when compared to Aβ1-40.

FIG. 3: Fluorescence intensity of thioflavin T

FIG. 3-1

The vertical axis represents the fluorescence intensity of thioflavin T, i.e., the content of β-sheet structure. The horizontal axis represents the incubation time. Solid square (▪), open square (□), solid triangle (▴) and solid circle (●) represent Aβ1-42, Aβ1-40, Aβ1-38 and Aβ1-37, respectively. In Aβ1-42, the fluorescence intensity of ThT was increased with increasing incubation time, whereas Aβ1-37, Aβ1-38 and Aβ1-40 showed no increase in the fluorescence intensity.

FIG. 3-2

This figure shows the fluorescence intensity of thioflavin T measured for a 1:3 mixture of Aβ1-42 and Aβ1-37, Aβ1-38 or Aβ1-40. The vertical axis represents the fluorescence intensity, i.e., the content of β-sheet structure. The horizontal axis represents the incubation time. Solid square (▪), open square (□), solid triangle (▴) and solid circle (●) represent Aβ1-42+buffer, Aβ1-42+Aβ1-40, Aβ1-42+Aβ1-38 and Aβ1-42+Aβ1-37, respectively.

FIG. 3-3

This figure shows a magnified view of FIG. 3-2 in the fluorescence intensity range between 0 and 6000000.

When compared to Aβ1-42 alone, the formation of β-sheet structure was inhibited in the presence of Aβ1-37, Aβ1-38 or Aβ1-40. The degree of inhibition was greater in the presence of Aβ1-37 and Aβ1-38 than in the presence of Aβ1-40. These results were well correlated with the results of CD analysis for β-sheet structure.

FIG. 4: Nerve cell toxicity of Aβ

The vertical axis represents MTT activity, expressed as a percentage of the control group (Aβ-untreated group). A smaller value means lower MTT activity and hence higher cell toxicity. Aβ1-42 showed a decrease in MTT activity, whereas Aβ1-37 showed no decrease.

FIG. 5: Results of MALDI-TOF/MS analysis for Aβ in rat fetal brain-derived nerve cell culture medium

FIG. 5-1

This figure shows the results of MALDI-TOF/MS analysis for each Aβ fragment in nerve cell culture supernatant in the absence of a test compound. The vertical axis represents the intensity and the horizontal axis represents the molecular weight. All mass data detected were corrected for the mass of human insulin and angiotensin III (5807.6 and 931.1, respectively), which were added as standards. The normalization of the detected Aβ intensity between samples was performed assuming that the detected intensity of internal standard Aβ12-28 was the same in all samples.

FIG. 5-2

This figure shows a magnified view of FIG. 5-1 in the molecular weight range between 2421 and 4565.

FIG. 6: Effects of individual compounds on Aβ fragments

The intensity of individual peaks was scored based on their area and normalized to the intensity of internal standard Aβ12-28 before being compared. The vertical axis represents the intensity of each Aβ fragment and individual columns represent the concentrations of a test compound added. The figure indicated that Aβ37 production was enhanced in a manner dependent on the concentration of the test compound.

FIG. 6-1: Compound A

FIG. 6-2: Compound B (CAS#501907-79-5)

FIG. 6-3: Compound C (CAS#670250-40-5)

FIG. 7: Quantitative results of ELISA assay for Aβ in rat fetal brain-derived nerve cell culture medium

The vertical axis represents the concentration of Aβ in a medium, expressed as a percentage of the control group (drug-untreated group), and the horizontal axis represents the concentration of a test compound added. Open square (□) and solid square (▪) represent Aβ40 and Aβ42, respectively. The figure indicated that both Aβ40 and Aβ42 production were inhibited in a manner dependent on the concentration of the test compound.

FIG. 7-1: Compound A

FIG. 7-2: Compound B (CAS#501907-79-5)

FIG. 7-3: Compound C (CAS#670250-40-5)

### DETAILED DESCRIPTION OF THE INVENTION

1. Summary of the Present Invention

In Aβ-based diseases, it has been found that Aβ40/42 accumulation induces the formation of amyloid plaques and causes various symptoms of the diseases. The present invention is based on the inventors' findings that enhanced production of Aβ37 prevents γ-secretase-mediated production of Aβ40/42 from APP and that Aβ37 and Aβ38 have an inhibitory effect against Aβ42 aggregation. Namely, the present invention relates to the therapeutic utility of a compound capable of enhancing Aβ37 production in the living body or a part thereof, or a compound capable of inhibiting Aβ40/42 production and enhancing Aβ37 production in the living body or a part thereof, or Aβ37 or Aβ38 in treating Aβ-based diseases such as Alzheimer's disease and Down's syndrome.

(1) Method for Inhibiting Aβ40/42 Production Characterized by Enhancing Aβ37 Production

The inventors of the present invention have clarified that enhanced production of Aβ37 prevents Aβ40/42 production. Thus, the present invention provides a method for inhibiting Aβ40/42 production, characterized by enhancing Aβ37 production in the living body or a part thereof. In the above method of the present invention, it is possible to use at least one member selected from the group consisting of a compound capable of enhancing Aβ37 production, and its salt and hydrates thereof, which are contemplated as being within the present invention.

The present invention also provides a method for identifying or screening such a compound capable of enhancing Aβ37 production. The above method of the present invention may be accomplished by comparing the amount of Aβ37 produced in the presence or absence of a candidate compound.

(2) Method for Inhibiting Aβ40/42 Production and Enhancing Aβ37 Production

The present invention provides a method for inhibiting Aβ40/42 production and enhancing Aβ37 production in the living body or a part thereof. In the above method of the present invention, it is possible to use at least one member selected from the group consisting of a compound capable of inhibiting Aβ40/42 production and enhancing Aβ37 production, and its salt and hydrates thereof, which are contemplated as being within the present invention.

The present invention also provides a method for identifying or screening such a compound capable of inhibiting Aβ40/42 production and enhancing Aβ37 production. The above method of the present invention may be accomplished by comparing the amount of each Aβ produced in the presence or absence of a candidate compound.

(3) Method for Inhibiting Aβ Aggregation

The inventors of the present invention have clarified that Aβ37 and Aβ38 are extremely less toxic to cells than Aβ40/42 and that Aβ37 and Aβ38 have an inhibitory effect against Aβ42 aggregation. Thus, the present invention provides a method for inhibiting Aβ aggregation, characterized by allowing Aβ37 and/or Aβ38 to act on Aβ40/42 in the living body or a part thereof.

The present invention also provides a method for inhibiting Aβ aggregation, characterized by enhancing Aβ37 or Aβ38 (preferably Aβ37) production in the living body or a part thereof. The present invention further provides a method for inhibiting Aβ aggregation, characterized by inhibiting Aβ40/42 production and enhancing Aβ37 or Aβ38 (preferably Aβ37) production in the living body or a part thereof. In the above methods of the present invention, it is possible to use at least one member selected from the group consisting of a compound capable of enhancing Aβ37 production, a compound capable of inhibiting Aβ40/42 production and enhancing Aβ37 production, and salts of the compounds and hydrates thereof. Aβ aggregation inhibitors containing these compounds are also contemplated as being within the present invention.

(4) Method for Preventing Nerve Cell Death

Previous studies have indicated that Aβ aggregation induces Aβ deposition on nerve cells and causes nerve cell death. The inventors of the present invention have clarified that enhanced production of Aβ37 inhibits Aβ40/42 production associated with such aggregation toxicity and that Aβ37 and Aβ38 have an inhibitory effect against Aβ42 aggregation. These effects prevent nerve cell death induced by Aβ aggregation. Thus, the present invention provides a method for preventing nerve cell death, characterized by allowing Aβ37 and/or Aβ38 to act on Aβ40/42 in the living body or a part thereof.

The present invention also provides a method for preventing nerve cell death, characterized by enhancing Aβ37 or Aβ38 (preferably Aβ37) production in the living body or a part thereof. The present invention further provides a method for preventing nerve cell death, characterized by inhibiting Aβ40/42 production and enhancing Aβ37 or Aβ38 (preferably Aβ37) production in the living body or a part thereof. In the above methods of the present invention, it is possible to use at least one member selected from the group consisting of a compound capable of enhancing Aβ37 production, a compound capable of inhibiting Aβ40/42 production and enhancing Aβ37 production, and salts of the compounds and hydrates thereof. Nerve cell death inhibitors containing these compounds are also contemplated as being within the present invention.

(5) Method for Treating Aβ-Based Diseases

The present invention provides a method for treating an Aβ-based disease. In the present invention, “treating an Aβ-based disease” includes preventing, slowing or reversing the progression of the disease. The treatment method of the present invention may be accomplished by administering to a mammal in need of treatment of the disease, an effective amount of a pharmaceutical composition containing at least one member selected from the group consisting of a compound capable of enhancing Aβ37 production, a compound capable of inhibiting Aβ40/42 production and enhancing Aβ37 production, and salts of the compounds and hydrates thereof. Such a pharmaceutical composition used for Aβ-based diseases is also contemplated as being within the present invention. Such a pharmaceutical composition is very effective in treating Aβ-based diseases because the compound(s) contained therein has an effect of enhancing Aβ37 production or an effect of enhancing Aβ37 production and inhibiting Aβ40/42 production, and also Aβ37 has an inhibitory effect against Aβ42 aggregation.

Alternatively, the method for treating an Aβ-based disease may be accomplished by administering to a mammal in need of treatment of the disease, an effective amount of a pharmaceutical composition containing at least one member selected from the group consisting of Aβ37, Aβ38, a polynucleotide encoding Aβ37 or Aβ38, and their salts and hydrates thereof. Such a pharmaceutical composition used for Aβ-based diseases is also contemplated as being within the present invention. Such a pharmaceutical composition is very effective in treating Aβ-based diseases because the contained Aβ37, Aβ38, a polynucleotide encoding Aβ37 or Aβ38, and their salts and hydrates thereof have an inhibitory effect against Aβ aggregation.

(6) Combination Therapy

The present invention may be accomplished by administering an effective amount of a pharmaceutical composition containing at least one member selected from the group consisting of a compound capable of enhancing Aβ37 production, a compound capable of inhibiting Aβ40/42 production and enhancing Aβ37 production, and salts of the compounds and hydrates thereof, as well as an effective amount of a pharmaceutical composition containing at least one member selected from the group consisting of a cholinesterase (ChE)-inhibiting substance, an NMDA (N-methyl-D-aspartate) receptor antagonist and an AMPA (α-amino-3-hydroxy-5-methyl-4-isoxazole propionic acid) receptor antagonist, which are administered separately or as a single pharmaceutical composition (blended formulation) containing these two ingredients. The present invention includes a method for treating an Aβ-based disease by combination therapy. Pharmaceutical compositions or kits used for combination therapy of Aβ-based diseases are also contemplated as being within the present invention. Such a pharmaceutical composition or kit is effective as a therapeutic agent for Aβ-based diseases. Moreover, such a kit may be used for detecting or predicting the effectiveness of a pharmaceutical composition for use in combination therapy, or may be used in a method for identifying or screening a compound suitable for a pharmaceutical composition for use in combination therapy.

The present invention will be described in more detail below.

As used herein, the term “living body” means a mammal and the term “part of the living body” encompasses various organs of the mammal, including the central nervous system (particularly brain, spinal cord) as well as living body-derived tissues, body fluids (including, e.g., blood, cerebrospinal fluid, lympha, saliva) or cells. Living body-derived cells also include cultured cells such as primary cultured cells and cultured cell lines.

As used herein, the term “mammal” means any animal which can be classified as a mammal, including human or non-human mammals (e.g., mouse, rat, hamster, guinea pig, rabbit, pig, dog, horse, cattle, monkey). Preferably, the mammal intended herein is a human.

As used herein, the term “APP” means β-amyloid precursor protein (βAPP). In the case of humans, it refers to a peptide that is encoded by the gene of the same name located in the long arm of chromosome 21 and that contains the Aβ region in its C-terminal segment.

APP is known to have isotypes. Table 1 shows the Accession numbers or Swiss prot Isoform IDs of human, mouse and rat APP isotypes, which are registered with GenBank or Swiss Prot. A representative isotype differs among species. For example, a representative isotype is APP770 amino acid isotype in humans, APP695 amino acid isotype in mice, and APP770 amino acid isotype in rats.

TABLE 1

| | GenBank accession number | | |
|---|---|---|---|
| | | Amino acid | |
| | cDNA sequence | sequence | (Swiss prot |
| Isotype | (SEQ ID NO) | (SEQ ID NO) | Isoform ID) |
| Human APP695 | NM_201414 | NP_958817 | (P05067-4) |
| | (SEQ ID NO: 1) | (SEQ ID NO: 2) | |
| Human APP751 | NM_201413 | NP_958816 | (P05067-8) |
| | (SEQ ID NO: 3) | (SEQ ID NO: 4) | |
| Human APP770 | NM_000484 | NP_000475 | (P05067-1) |
| | (SEQ ID NO: 5) | (SEQ ID NO: 6) | |
| | | | P05067 |
| Mouse APP695 | NM_007471 | NP_031497 | (P12023-2) |
| | (SEQ ID NO: 7) | (SEQ ID NO: 8) | |
| Mouse APP751 | n/a | n/a | (P12023-3) |
| Mouse APP770 | AY267348 | AAP23169 | (P12023-1) |
| | | | P12023 |
| Rat APP695 | n/a | n/a | (P08592-2) |
| Rat APP751 | n/a | n/a | (P08592-7) |
| Rat APP770 | NM_019288 | NP_062161 | (P08592-1) |
| | (SEQ ID NO: 9) | (SEQ ID NO: 10) | |
| | | | P08592 |

As used herein, the term “Aβ” means β-amyloid protein, amyloid β protein, β-amyloid peptide, amyloid β peptide or amyloid beta. For example, Aβ refers to any peptide composed of about 33 to about 44 amino acid residues in human APP695 amino acid isotype, which preferably contains all or part of amino acid residues at positions 597 to 640 of APP and which is produced from APP by N-terminal proteolysis and subsequent C-terminal proteolysis.

As used herein, the term “γ-secretase” means an enzyme or a complex of multiple molecules that cleaves (degrades) APP within its transmembrane region to drive Aβ production.

When expressed herein as “Aβ37”, “Aβ38”, “Aβ40” and “Aβ42”, they mean AβX-37, AβX-38, AβX-40 and AβX-42 (wherein X is an integer of 1 to 17), respectively. Since X is preferably 1 or 11, X represents 1 or 11 unless otherwise specified.

More specifically, as used herein, the term “Aβ37” refers to a peptide that is derived from Aβ37 composed of 37 amino acid residues by deletion of the N-terminal X-1 residues, i.e., that covers amino acids X to 37. The term “Aβ40/42” means Aβ40 and Aβ42. Aβ40 refers to a peptide that is derived from Aβ40 composed of 40 amino acid residues by deletion of the N-terminal X-1 residues, i.e., that covers amino acids X to 40. Aβ42 refers to a peptide that is derived from Aβ42 composed of 42 amino acid residues by deletion of the N-terminal X-1 residues, i.e., that covers amino acids X to 42. X represents an integer of 1 to 17 and, unless otherwise specified, X is 1 or 11.

As used herein, the phrase “enhance Aβ37 production” or “enhancing Aβ37 production” means an effect of increasing the level of Aβ37 production.

As used herein, the phrase “inhibiting Aβ40/42 production” means an effect of decreasing (reducing) the level of Aβ40/42 production or stopping Aβ40/42 production.

As used herein, the phrase of effect of “inhibiting Aβ40/42 production and enhancing Aβ37 production” means an effect of not only decreasing (reducing) the level of Aβ40/42 production or stopping Aβ40/42 production, but also increasing the level of Aβ37 production.

As used herein, the term “compound capable of enhancing Aβ37 production” may refer to any compound as long as it has an effect of enhancing Aβ37 production.

As used herein, the term “compound capable of inhibiting Aβ40/42 production and enhancing Aβ37 production” may refer to any compound as long as it has an effect of inhibiting Aβ40/42 production and enhancing Aβ37 production.

As used herein, the phrases “inhibiting production” and “inhibited production” or “enhancing production” and “enhanced production” mean reproducible changes in production levels. For example, the change in production level may be any value as long as it means an increase or decrease of, for example, 1%, 5%, 10%, 20%, 40%, or 40% or more.

As used herein, the term “compound” refers to one or more compounds contained in, e.g., expression products of gene libraries, natural or synthetic low-molecular compound libraries, nucleic acids (oligo DNAs, oligo RNAs), natural or synthetic peptide libraries, antibodies, substances released from bacteria (including substances released by bacterial metabolism), cell (microorganism, plant cell or animal cell) extracts, cell (microorganism, plant cell or animal cell) culture supernatants, purified or partially purified peptides, marine organisms, plant- or animal-derived extracts, soil, and random phage peptide display libraries. Such a compound may be either a novel or a known compound. Moreover, such a compound may be modified by existing chemical means, physical means and/or biochemical means. For example, it may be subjected to direct chemical modification (e.g., acylation, alkylation, esterification, amidation) or random chemical modification to convert into a structural analog. Such a compound may also be one that is identified by, e.g., pharmacophore search for the compound or computer-aided structure comparison programs.

As used herein, the term “derivative” means a compound obtained by partial alteration of the original compound. The term “derivative” also includes products obtained by addition reaction.

As used herein, the term “salt” refers to a pharmaceutically acceptable salt, which is not limited in any way as long as a pharmaceutically acceptable salt can be formed with the compound or its equivalent used in the present invention, the peptide or its equivalent used in the present invention or the polynucleotide or its equivalent used in the present invention serving as a therapeutic agent for Aβ-based diseases. More specifically, preferred examples include halogenated hydroacid salts (e.g., hydrofluoride salt, hydrochloride salt, hydrobromide salt, hydroiodide salt), inorganic acid salts (e.g., sulfate salt, nitrate salt, perchlorate salt, phosphate salt, carbonate salt, bicarbonate salt), organic carboxylic acid salts (e.g., acetate salt, oxalate salt, maleate salt, tartrate salt, fumarate salt, citrate salt), organic sulfonic acid salts (e.g., methanesulfonate salt, trifluoromethanesulfonate salt, ethanesulfonate salt, benzenesulfonate salt, toluenesulfonate salt, camphorsulfonate salt), amino acid salts (e.g., aspartate salt, glutamate salt), quaternary amine salts, alkali metal salts (e.g., sodium salt, potassium salt), and alkaline earth metal salts (e.g., magnesium salt, calcium salt).

2. Method for Inhibiting Aβ40/42 Production Characterized by Enhancing Aβ37 Production

The present invention provides a method for inhibiting Aβ40/42 production, characterized by enhancing Aβ37 production in the living body or a part thereof. The above method may be accomplished by using at least one member selected from the group consisting of a compound capable of enhancing Aβ37 production, and its salt and hydrates thereof. The method of the present invention is based on a mechanism in which enhanced production of Aβ37 results in inhibition of Aβ40/42 production. The compound capable of enhancing Aβ37 production achieves inhibition of Aβ40/42 production as a result of enhanced production of Aβ37.

As used herein, the phrase “the compound or its equivalent used in the present invention” is intended to comprise at least one member selected from the group consisting of the above compound capable of enhancing Aβ37 production, and its salt and hydrates thereof.

Namely, the compound or its equivalent used in the present invention comprises at least one member selected from:
(i) a compound capable of enhancing Aβ37 production;
(ii) a salt of (i) above;
(iii) a hydrate of (i) above;
(iv) a hydrate of (ii) above; and
(v) a combination of (i) to (iv) above.

Preferred examples of the compound or its equivalent used in the present invention include at least one member selected from the group consisting of the compounds and their derivatives described in the Example section below, and their salts and hydrates thereof. Compounds specifically exemplified include:
(E)-N-biphenyl-3-ylmethyl-3-[3-methoxy-4-(4-methylimidazol-1-yl)phenyl]-acrylamide (hereinafter referred to as “Compound A”);
a compound designated CAS#501907-79-5 (hereinafter referred to as “Compound B”); and
a compound designated CAS#670250-40-5 (hereinafter referred to as “Compound C”).

The compound or its equivalent used in the present invention is characterized in that it has an effect of enhancing Aβ37 production and, as a result, inhibits Aβ40/42 production. The strength of its effect of enhancing Aβ37 production or inhibiting Aβ40/42 production will not affect the utility of the present invention.

The compound or its equivalent used in the present invention may further have an effect of enhancing Aβ38 or Aβ39 production.

The compound or its equivalent used in the present invention, i.e., at least one member selected from the group consisting of a compound capable of enhancing Aβ37 production, and its salt and hydrates thereof may be prepared by known manufacturing procedures if it is a known compound, may be obtained by known extraction or purification procedures if it is a naturally-occurring compound, or may be purchased if it is commercially available. Moreover, derivatives and other forms of known compounds may be modified by chemical means, physical means and/or biochemical means.

Compounds A, B and C mentioned above may be prepared by, but not limited to, such as the procedures described in the Example section.

The present invention also provides a method for identifying or screening such a compound capable of enhancing Aβ37 production. The above method of the present invention may be accomplished by comparing the amount of Aβ37 in the presence or absence of a candidate compound. The details of the identification or screening method will be described later in “6. Method for identifying or screening the compound or its equivalent used in the present invention.”

3. Method for Inhibiting Aβ40/42 Production and Enhancing Aβ37 Production

The present invention provides a method for inhibiting Aβ40/42 production and enhancing Aβ37 production. The above method may be accomplished by using at least one member selected from the group consisting of a compound capable of inhibiting Aβ40/42 production and enhancing Aβ37 production, and its salt and hydrates thereof.

As used herein, the phrase “the compound or its equivalent used in the present invention” is intended to comprise at least one member selected from the group consisting of the above compound capable of inhibiting Aβ40/42 production and enhancing Aβ37 production, and its salt and hydrates thereof.

Namely, the compound or its equivalent used in the present invention comprises, in addition to at least one member selected from the group consisting of a compound capable of enhancing Aβ37 production, and its salt and hydrates thereof (i.e., (i) to (v) listed above in “2. Method for inhibiting Aβ40/42 production characterized by enhancing Aβ37 production”), at least one member selected from:
(vi) a compound capable of inhibiting Aβ40/42 production and enhancing Aβ37 production;
(vii) a salt of (vi) above;
(viii) a hydrate of (vi) above;
(ix) a hydrate of (vii) above; and
(x) a combination of (vi) to (ix) above.

Preferred examples of the compound or its equivalent used in the present invention include at least one member selected from the group consisting of the compounds and their derivatives described in the Example section below, and their salts and hydrates thereof. Compounds specifically exemplified include:
(E)-N-biphenyl-3-ylmethyl-3-[3-methoxy-4-(4-methylimidazol-1-yl)phenyl]-acrylamide (hereinafter referred to as “Compound A”);
a compound designated CAS#501907-79-5 (hereinafter referred to as “Compound B”); and
a compound designated CAS#670250-40-5 (hereinafter referred to as “Compound C”).

The compound or its equivalent used in the present invention is characterized in that it has an effect of inhibiting Aβ40/42 production and enhancing Aβ37 production. The strength of its effect of inhibiting Aβ40/42 production and the strength of its effect of enhancing Aβ37 production will not affect the utility of the present invention.

The compound or its equivalent used in the present invention may further have an effect of enhancing Aβ38 or Aβ39 production.

The compound or its equivalent used in the present invention, i.e., at least one member selected from the group consisting of a compound capable of inhibiting Aβ40/42 production and enhancing Aβ37 production, and its salt and hydrates thereof may be prepared by known manufacturing procedures if it is a known compound, may be obtained by known extraction or purification procedures if it is a naturally-occurring compound, or may be purchased if it is commercially available. Moreover, derivatives and other forms of known compounds may be modified by chemical means, physical means and/or biochemical means.

Compounds A, B and C mentioned above may be prepared by, but not limited to, such as the procedures described in the Example section.

The present invention also provides a method for identifying or screening such a compound capable of inhibiting Aβ40/42 production and enhancing Aβ37 production. The above method of the present invention may be accomplished by comparing the amount of each Aβ in the presence or absence of a candidate compound. The details of the identification or screening method will be described later in “6. Method for identifying or screening the compound or its equivalent used in the present invention.”

4. Method for Inhibiting Aβ Aggregation

As described above, the inventors of the present invention have clarified that Aβ37 and Aβ38 are extremely less toxic to cells than Aβ40/42 and that Aβ37 and Aβ38 have an inhibitory effect against Aβ42 aggregation. Thus, the present invention provides a method for inhibiting Aβ aggregation, characterized by allowing Aβ37 or Aβ38 to act on Aβ42 in the living body or a part thereof. In the above method, Aβ37 or Aβ38 which is allowed to act on Aβ42 may be endogenous one produced by the action of the compound or its equivalent used in the present invention or may be exogenous one. The compound or its equivalent used in the present invention may further have an effect of enhancing Aβ38 or Aβ39 production. Moreover, it is also possible to allow Aβ37 and Aβ38 to simultaneously act on Aβ42.

The above phrase “allowing Aβ37 or Aβ38 to act on Aβ42” means that Aβ42 is treated with Aβ37 or Aβ38. Procedures for this treatment are not limited, and any procedure can be selected for this purpose. By way of example, Aβ42 may be contacted with Aβ37 or Aβ38, or alternatively, Aβ42 may be placed together with Aβ37 or Aβ38 in a single system (e.g., in a single test tube).

As used herein, “endogenous” Aβ37 or Aβ38 refers to Aβ37 or Aβ38 derived from the living body or a part thereof, or alternatively refers to Aβ37, Aβ38, a salt thereof, a hydrate thereof or a combination thereof, which is produced in the living body or a part thereof.

As described above, Aβ37 or Aβ38 produced in the living body or a part thereof by the action of the compound or its equivalent used in the present invention, which is capable of enhancing Aβ37 production, is also included in endogenous Aβ37 or Aβ38. In the above method, it is therefore possible to use the compound or its equivalent used in the present invention, which may be used as an Aβ aggregation inhibitor. As described above, Aβ37 or Aβ38 produced in the living body or a part thereof by the action of the compound or its equivalent used in the present invention, which is capable of inhibiting Aβ40/42 production and enhancing Aβ37 production, is also included in endogenous Aβ37 or Aβ38. In the above method, it is therefore possible to use the compound or its equivalent used in the present invention, which may be used as an Aβ aggregation inhibitor.

As used herein, “exogenous” Aβ37 or Aβ38 refers to Aβ37, Aβ38, a salt thereof, a hydrate thereof or a combination thereof, which is derived from any origin other than the living body or produced elsewhere other than the living body. It also includes embodiments where Aβ37 or Aβ38 is prepared from a polynucleotide encoding Aβ37 or Aβ38, a salt thereof, a hydrate thereof or a combination thereof. The details of exogenous Aβ37 or Aβ38 will be described later in “7. Aβ37 or Aβ38.”

Since enhanced production of Aβ37 or Aβ38 inhibits Aβ aggregation, the present invention also includes a method for inhibiting Aβ aggregation, characterized by enhancing Aβ37 or Aβ38 (preferably Aβ37) production in the living body or a part thereof. Likewise, since enhanced production of Aβ37 or Aβ38 inhibits Aβ aggregation, the present invention also includes a method for inhibiting Aβ aggregation, characterized by inhibiting Aβ40/42 production and enhancing Aβ37 or Aβ38 (preferably Aβ37) production in the living body or a part thereof. In the above methods of the present invention, it is possible to use the compound or its equivalent used in the present invention, i.e., at least one member selected from the group consisting of a compound capable of enhancing Aβ37 production, a compound capable of inhibiting Aβ40/42 production and enhancing Aβ37 production, and salts of the compounds and hydrates thereof. The compound or its equivalent used in the present invention may further have an effect of enhancing Aβ38 or Aβ39 production.

The present invention includes an Aβ aggregation inhibitor containing the compound or its equivalent used in the present invention and an Aβ aggregation inhibitor containing the above exogenous Aβ37 or Aβ38, both inhibitors being used in the method for inhibiting Aβ aggregation.

5. Method for Preventing Nerve Cell Death

Previous studies have indicated that Aβ aggregation induces Aβ deposition on nerve cells and causes nerve cell death. The inventors of the present invention have clarified that enhanced production of Aβ37 inhibits Aβ40/42 production associated with such aggregation toxicity and that Aβ37 and Aβ38 have an inhibitory effect against Aβ42 aggregation. These effects prevent nerve cell death induced by Aβ aggregation. Thus, the present invention provides a method for preventing nerve cell death, characterized by allowing Aβ37 or Aβ38 to act on Aβ42 in the living body or a part thereof. In the above method, Aβ37 or Aβ38 which is allowed to act on Aβ42 may be endogenous one produced by the action of the compound or its equivalent used in the present invention or may be exogenous one. The compound or its equivalent used in the present invention may further have an effect of enhancing Aβ38 or Aβ39 production. Moreover, it is also possible to allow Aβ37 and Aβ38 to simultaneously act on Aβ42.

The above phrase “allowing Aβ37 or Aβ38 to act on Aβ42” means that Aβ42 is treated with Aβ37 or Aβ38. Procedures for this treatment are not limited, and any procedure can be selected for this purpose. By way of example, Aβ42 may be contacted with Aβ37 or Aβ38, or alternatively, Aβ42 may be placed together with Aβ37 or Aβ38 in a single system (e.g., in a single test tube).

As described above, Aβ37 or Aβ38 produced in the living body or a part thereof by the action of the compound or its equivalent used in the present invention, which is capable of enhancing Aβ37 production, is also included in endogenous Aβ37 or Aβ38. In the above method, it is therefore possible to use the compound or its equivalent used in the present invention, which may be used as a nerve cell death inhibitor. As described above, Aβ37 or Aβ38 produced in the living body or a part thereof by the action of the compound or its equivalent used in the present invention, which is capable of inhibiting Aβ40/42 production and enhancing Aβ37 production, is also included in endogenous Aβ37 or Aβ38. In the above method, it is therefore possible to use the compound or its equivalent used in the present invention, which may be used as a nerve cell death inhibitor.

The details of exogenous Aβ37 or Aβ38 will be described later in “7. Aβ37 or Aβ38.”

Since enhanced production of Aβ37 or Aβ38 inhibits Aβ aggregation, the present invention also includes a method for preventing nerve cell death, characterized by enhancing Aβ37 or Aβ38 (preferably Aβ37) production in the living body or a part thereof. Likewise, since enhanced production of Aβ37 or Aβ38 inhibits Aβ aggregation, the present invention also includes a method for preventing nerve cell death, characterized by inhibiting Aβ40/42 production and enhancing Aβ37 or Aβ38 (preferably Aβ37) production in the living body or a part thereof. In the above methods of the present invention, it is possible to use the compound or its equivalent used in the present invention, i.e., at least one member selected from the group consisting of a compound capable of enhancing Aβ37 production, a compound capable of inhibiting Aβ40/42 production and enhancing Aβ37 production, and salts of the compounds and hydrates thereof. The compound or its equivalent used in the present invention may further have an effect of enhancing Aβ38 or Aβ39 production.

The present invention includes a nerve cell death inhibitor containing the compound or its equivalent used in the present invention and a nerve cell death inhibitor containing the above exogenous Aβ37 or Aβ38, both inhibitors being used in the method for preventing nerve cell death.

The nerve cells mentioned above include cells of the central nervous system, such as brain-derived nerve cells, preferably brain cortex-derived nerve cells. More preferably, these cells are of mammalian origin. Likewise, brain cortex-derived primary cultured nerve cells are also among the intended nerve cells.

6. Method for Identifying or Screening the Compound or its Equivalent used in the Present Invention

Among members of the compound or its equivalent used in the present invention, a compound capable of enhancing Aβ37 production can also be obtained by identifying its effect of enhancing Aβ37 production using standard procedures for identification or screening in the art, as shown below. Likewise, among members of the compound or its equivalent used in the present invention, a compound capable of inhibiting Aβ40/42 production and enhancing Aβ37 production can also be obtained by identifying its effect of inhibiting Aβ40/42 production and enhancing Aβ37 production using standard procedures for identification or screening, as shown below. For these procedures, various identification or screening techniques can be adapted as appropriate, ranging from small-scale techniques for handling a small number of candidate compounds to large-scale techniques for handling a large number of candidate compounds.

To confirm whether or not a candidate compound has an effect of enhancing Aβ37 production or an effect of inhibiting Aβ40/42 production and enhancing Aβ37 production, a biological composition may be treated with the candidate compound, and the presence or absence of or changes in the amounts of individual Aβs, which are proteolysis products of APP, may be measured and compared in the presence or absence of the candidate compound. For example, the presence or absence of or changes in the amounts of individual Aβs may be measured using standard antibody assays, such as immunoprecipitation, ELISA (enzyme-linked immunosorbent assay), Western blotting and radioimmunoassay. Alternatively, immunoprecipitation may be combined with MALDI-TOF or MALDI-TOF/MS. In these assays, antibody molecules may be labeled for direct detection (using, e.g., a radioisotope, an enzyme, a fluorescent agent, a chemiluminescent agent) or may be used in combination with a secondary antibody or reagent which detects binding (e.g., a combination of biotin and horseradish peroxidase-conjugated avidin, a secondary antibody conjugated with a fluorescent compound such as fluorescein, rhodamine or Texas Red). Alternatively, each Aβ may also be quantified using known techniques, for example, by MALDI-TOF/MS (described later) using a calibration curve prepared with internal standards.

Namely, the present invention provides the methods illustrated in (1) and (2) below, which are hereinafter also referred to as “the identification method of the present invention” or “the screening method of the present invention.”

(1) The present invention provides the following method for identifying or screening a compound capable of enhancing Aβ37 production.

A method for identifying or screening a compound capable of enhancing Aβ37 production, which comprises:
(a) contacting a candidate compound with a biological composition;
(b) measuring the amount of Aβ37 in the biological composition contacted with the candidate compound and the amount of Aβ37 in a biological composition not contacted with the candidate compound;
(c) selecting a candidate compound that produces an increase in the amount of Aβ37 in the biological composition contacted with the candidate compound when compared to the amount of Aβ37 in the biological composition not contacted with the candidate compound; and
(d) identifying the candidate compound obtained in (c) above as a compound capable of enhancing Aβ37 production.

The above method of the present invention may be accomplished by comparing the amount of Aβ37 produced in the presence or absence of a candidate compound.

(2) The present invention provides the following method for identifying or screening a compound capable of inhibiting Aβ40/42 production and enhancing Aβ37 production.

A method for identifying or screening a compound capable of inhibiting Aβ40/42 production and enhancing Aβ37 production, which comprises:
(a) contacting a candidate compound with a biological composition;
(b) measuring the amounts of Aβ40/42 and Aβ37 in the biological composition contacted with the candidate compound and the amounts of Aβ40/42 and Aβ37 in a biological composition not contacted with the candidate compound;
(c) selecting a candidate compound that causes a reduction in the amount of Aβ40/42 and also produces an increase in the amount of Aβ37 in the biological composition contacted with the candidate compound when compared to the amounts of Aβ40/42 and Aβ37 in the biological composition not contacted with the candidate compound; and
(d) identifying the candidate compound obtained in (c) above as a compound capable of inhibiting Aβ40/42 production and enhancing Aβ37 production.

The above method of the present invention may be accomplished by comparing the amount of each Aβ (e.g., Aβ37, Aβ40, Aβ42) produced in the presence or absence of a candidate compound.

As used herein, the term “contacting” means that a candidate compound and a biological composition are reacted with each other in order to produce Aβ37 in the biological composition by the action of the candidate compound.

The phrase “the amount of Aβ37 in a biological composition not contacted with the candidate compound” or “the amounts of Aβ40/42 and Aβ37 in a biological composition not contacted with the candidate compound” means serving as a control.

The phrase “produce an increase” means that the amount of Aβ37 in a biological composition is increased by contact with a candidate compound when compared to a control.

The phrase “cause a reduction” means that the amount of Aβ40/42 in a biological composition is reduced by contact with a candidate compound when compared to a control.

The term “biological composition” means any composition containing γ-secretase and APP, including reconstructed cell-free systems, cells, transgenic non-human animals engineered to overexpress APP (hereinafter referred to as “APP transgenic non-human animal”) and non-transgenic non-human animals. Cells in this context may be nerve cells including cells of the central nervous system, such as brain-derived nerve cells, preferably brain cortex-derived nerve cells, and more preferably brain cortex-derived primary cultured nerve cells. These cells are preferably of mammalian origin. The details of how to prepare such primary cultured nerve cells and APP transgenic non-human animals will be described later. The term “APP-expressing cells” means cells endogenously expressing APP or cells forced to express APP.

For the purpose of the present invention, γ-secretase and APP may be either endogenous or exogenous. Endogenous γ-secretase and APP mean those derived from the living body or a part thereof, which may remain contained in the living body or a part thereof or may be γ-secretase and APP fractions of cell lysate. The cell lysate may be prepared from γ-secretase- and APP-containing cells, for example, by solubilization with a hypotonic solution or a detergent, or by ultrasonic disruption or physical disruption. In some cases, the cell lysate may be subjected to a purification means such as a column. Exogenous γ-secretase or APP means γ-secretase- or APP-expressing cells engineered to express β-secretase or APP using each vector containing a polynucleotide encoding each molecule constituting γ-secretase or a vector containing a polynucleotide encoding APP. Alternatively, it means a γ-secretase or APP fraction of cell lysate from these γ-secretase- or APP-expressing cells. The cell lysate may be prepared from γ-secretase- and APP-containing cells, for example, by solubilization with a hypotonic solution or a detergent, or by ultrasonic disruption or physical disruption. In some cases, the cell lysate may be subjected to a purification means such as a column. The vector(s) used for this purpose may be transfected into cells to induce transient gene expression, or may be integrated into the cellular genome to ensure stable gene expression. Host cells to be transfected with such a vector may be those capable of gene expression. Examples of mammalian cells include Chinese hamster ovary (CHO) cells, fibroblasts and human glioma cells.

Preparation of Primary Cultured Nerve Cells

As described above, in the present invention, it is possible to use, as a biological composition, nerve cells including cells of the central nervous system, such as brain-derived nerve cells, preferably brain cortex-derived nerve cells, and more preferably brain cortex-derived primary cultured nerve cells. The preparation of brain cortex-derived primary cultured nerve cells will be illustrated below, but is not limited to this example.

After pregnant animals (e.g., rats, mice) are anesthetized with ether or the like, fetuses (16 to 21 days of embryonic age) are aseptically extracted from the pregnant animals. Brains are extracted from the above fetuses and immersed in ice-cold L-15 medium. Brain cortices are collected under a stereoscopic microscope. Pieces of each brain region are enzymatically treated in an enzyme solution containing trypsin and DNase to disperse cells. The enzymatic reaction is stopped by addition of horse serum or the like. After centrifugation, the supernatant is removed and a medium is added to cell pellets. The medium used for this purpose may be, for example, a serum-free medium developed for long-term maintenance of hippocampal cell culture and/or central nervous system cell culture (e.g., adult nerve cell culture), which may be supplemented with auxiliary reagents to ensure longer-term survival of the nerve cells (Brewer, G. J., J. Neurosci. Methods, 71, 45, 1997, Brewer, G. J., et al., J. Neurosci. Res., 35, 567, 1993). For example, preferred is Neurobasal™ medium (Invitrogen Corporation) supplemented with 1% to 5%, preferably 2% of auxiliary reagents, and more preferred is Neurobasal™ medium supplemented with 2% B-27 supplement (Invitrogen Corporation), 0.5 mM L-glutamine, Antibiotics and Antimycotics as auxiliary reagents (hereinafter also referred to as “Neurobasal/B27”). Particularly preferred is Neurobasal™ medium supplemented with 2% B-27 supplement, 25 μM 2-mercaptoethanol (2-ME), 0.5 mM L-glutamine, Antibiotics and Antimycotics (hereinafter also referred to as “Neurobasal/B27/2ME”). The cells are dispersed again by pipetting and then filtered to remove cell aggregates, thereby obtaining a nerve cell suspension. The nerve cell suspension is diluted with the medium and the cells are seeded in culture plates at a uniform density. After the cells are cultured for 1 day under given conditions (e.g., in an incubator atmosphere of 5% CO₂, 95% air and 37° C.), the medium is entirely replaced by fresh Neurobasal/B27/2ME mentioned above.

Transgenic Non-Human Animal Model

As described above, in the present invention, it is possible to use an APP transgenic non-human animal as a biological composition. Namely, whether or not a candidate compound or the compound or its equivalent used in the present invention has an effect of enhancing Aβ37 production or an effect of inhibiting Aβ40/42 production and enhancing Aβ37 production may be confirmed by screening using an APP transgenic non-human animal model. APP transgenic non-human animal models are well known in the art, exemplified by Tg2576 mice described in J. Neurosci. 21(2), 372-381, 2001 and J. Clin. Invest., 112, 440-449, 2003. Namely, an example will be given below of test procedures using Tg2576 mice. By measuring the amount of each Aβ in the brain, cerebrospinal fluid or serum of Tg2576 mice receiving a γ-secretase inhibitor N-[N-(3,5-difluorophenacetyl)-L-alanyl]-S-phenylglycine t-butyl ester or a candidate compound, the compound or its equivalent used in the present invention, etc. (J. Pharmacol. Exp. Ther. 305, 864-871, 2003), it is possible to evaluate whether or not the above compound has an effect of enhancing Aβ37 production or an effect of inhibiting Aβ40/42 production and enhancing Aβ37 production.

In the present invention, APP transgenic non-human animals may be of any species, including mouse, rat, guinea pig, hamster, rabbit, dog, cat, goat, cattle or horse.

Non-Transgenic Non-Human Animal Model

As described above, in the present invention, it is possible to use a non-transgenic non-human animal model as a biological composition. Namely, whether or not a candidate compound or the compound or its equivalent used in the present invention has an effect of enhancing Aβ37 production or an effect of inhibiting Aβ40/42 production and enhancing Aβ37 production may be confirmed by screening using non-transgenic non-human animals. By way of example, there is a report of a method for measuring the amount of Aβ in the cerebrospinal fluid of guinea pigs receiving simvastatin (PNAS, 98, 5856-5861, 2001) or a method for measuring the amount of Aβ40 in the cerebrospinal fluid of rats receiving a γ-secretase inhibitor LY411575 (JPET, 313, 902-908, 2005). Thus, in accordance with these methods, by measuring the amount of each Aβ in the brain, cerebrospinal fluid or blood of a non-transgenic non-human animal model (e.g., guinea pig, mouse, rat) receiving a candidate compound or the compound or its equivalent used in the present invention, it is possible to evaluate whether or not the candidate compound has an effect of enhancing Aβ37 production or an effect of inhibiting Aβ40/42 production and enhancing Aβ37 production.

To illustrate the identification or screening method of the present invention, an example using MALDI-TOF/MS will be given below.

Analysis of Aβ3 by MALDI-TOF/MS [Matrix-Associated Laser Desorption Ionization-Time of Flight/Mass Spectrometry]

In this specification, MALDI-TOF/MS may be performed as described in, e.g., Rong Wang, David Sweeney, Sammuel E. Gangy, Sangram S. Sisodia, J. of Biological Chemistry, 271, (50), 31894-31902, 1996, Takeshi Ikeuchi, Georgia Dolios, Seong-Hun Kim, Rong Wang, Sangram S. Sisodia, J. of Biological Chemistry, 278, (9), 7010-7018, 2003, Sascha Weggen, Jason L. Erikson, Pritam Das, Sarah Sagi, Rong Wang, Claus U. Pietrzik, Kirk A. Findlay, Tawnya E. Smith, Michael P. Murphy, Thomas Bulter, David E. Kang, Numa Marquez-sterling, Todd E. Golde, Edward H. Koo, Nature, 414, 212-216, 2001, and Masayasu Okochi, et al., Idenshi Igaku (Gene Medicine), Vol. 7 (1), 12-16, 2003. More specifically, MALDI-TOF/MS may be performed as follows.

For MALDI-TOF/MS analysis, it is possible to use cells of the central nervous system, preferably brain-derived cells, preferably brain cortex-derived nerve cells, and more preferably brain cortex-derived primary cultured nerve cells. Brains are extracted from non-human animals and nerve cells may be prepared from the extracted brains in a routine manner. The medium used for this purpose may be, for example, a serum-free medium developed for long-term maintenance of hippocampal cell culture and/or central nervous system cell culture (e.g., adult nerve cell culture), which may be supplemented with auxiliary reagents to ensure longer-term survival of the nerve cells (Brewer, G. J., J. Neurosci. Methods, 71, 45, 1997, Brewer, G. J., et al., J. Neurosci. Res., 35, 567, 1993). An example is Neurobasal™ medium (Invitrogen Corporation) supplemented with, e.g., 1% to 5%, preferably 2% of auxiliary reagents and 10 to 30 μM, preferably 25 μM of 2-mercaptoethanol (2-ME). Preferred is Neurobasal™ medium (Invitrogen Corporation) supplemented with 2% B-27 supplement (Invitrogen Corporation), 25 μM 2-mercaptoethanol (2-ME), 0.5 mM L-glutamine, Antibiotics and Antimycotics (Neurobasal/B27/2ME). For use in assay, the above medium is preferably free from 2-ME (Neurobasal/B27). Several days after culturing the cells, the medium is removed and replaced by Neurobasal/B27. Then, a candidate compound in a vehicle (e.g., an aprotic polar solvent, preferably DMSO (dimethyl sulfoxide)) is diluted with Neurobasal/B-27 and added to the cells and mixed then. The final concentration of the vehicle (e.g., an aprotic polar solvent, preferably DMSO) is preferably kept at 1% or below. On the other hand, the control group may receive the vehicle (e.g., an aprotic polar solvent, preferably DMSO) alone. After culturing for several days in the presence of the candidate compound or vehicle (e.g., an aprotic polar solvent, preferably DMSO), the whole volume of the medium may be used as a MALDI-TOF/MS sample. Cell survival may be evaluated in a known manner, for example, by MTT assay described later.

Next, Aβ fragments may be collected, e.g., by immunoprecipitation. Each sampled culture supernatant is collected and centrifuged to sediment cell fragments. The supernatant may be supplemented with, as an internal standard, synthetic Aβ (e.g., synthetic Aβ12-28 (Bachem)) available to those skilled in the art. The supernatant is further supplemented with a desired anti-Aβ antibody (preferably an anti-Aβ monoclonal antibody, such as a clone under the name 4G8, Signet Laboratories, Inc) available to those skilled in the art, followed by addition of and mixing with a Protein G- and/or Protein A-conjugated water-insoluble resin such as sepharose or agarose (e.g., Protein G plus Protein A Agarose), which has been blocked with BSA or the like according to routine procedures. The anti-Aβ antibody used for this purpose may be an anti-human Aβ monoclonal antibody. Although a commercially available anti-human Aβ monoclonal antibody may be used, those skilled in the art will be able to readily prepare such an antibody. For example, animals (e.g., mice) are first immunized once to three times using a sequence common to Aβs as an antigen, and immunocompetent cells are collected from the animals and immortalized by cell fusion or other techniques to give monoclonal antibody-producing cells. The resulting monoclonal antibody-producing cells are administered intraperitoneally to nude mice or the like. A monoclonal antibody of interest can be obtained from the collected peritoneal fluid, but antibody preparation is not limited to the above procedures. An Aβ sequence used as an antigen can be appropriately selected by those skilled in the art. In addition, a peptide segment used as an antigen may be, but not limited to, a peptide which is naturally occurring, synthesized with an automatic synthesizer, prepared from APP in a biochemical manner, or commercially available.

The immunoprecipitated Aβ fragments may be obtained by collection using a Protein G- and/or Protein A-conjugated water-insoluble resin (e.g., Protein G plus Protein A Agarose), washing in a routine manner and elution of Aβs. The elution may be accomplished as follows: after washing with ion exchanged water, as much fluid as possible is removed and Aβs are eluted with, for example, a solution containing 0.2% NOG, 2.4% trifluoroacetic acid (TFA; PIERCE) and 48.7% acetonitrile (HPLC grade, Wako Pure Chemical Industries, Ltd.). However, the elution is not limited to the above procedures and those skilled in the art will be able to elute Aβs on the basis of known techniques.

Each Aβ eluate and a matrix solution are spotted at the same position on a sample plate for mass spectrometry and air-dried at room temperature, followed by analysis with a mass spectrometer. The matrix solution used for this purpose may be a solution commonly used for MALDI-TOS/MS, such as prepared by dissolving α-cyano-4-hydroxy-cinnamic acid (CHCA; BRUKER DALTONICS) into 0.2% NOG, 0.1% TFA and 33% acetonitrile at a saturating concentration and then adding thereto insulin and angiotensin III as mass standards. In addition to these substances, any peptide or compound may be used as a mass standard as long as its molecular weight is outside of the molecular weight range (about 3,000 to 4514) of each Aβ to be analyzed. For example, insulin may be replaced by ω-Agatoxin TK (molecular weight: 5273.0), human Adrenomedullin 2 (molecular weight: 5100.7) or the like, and angiotensin III may be replaced by Angiotensin II (molecular weight: 1046.2), human Endokinin D (molecular weight: 1574.8) or the like.

Those skilled in the art will be able to readily conduct Aβ measurement by MALDI-TOF/MS in accordance with operational procedures for measuring instruments. Examples of instruments available for use include, but are not limited to, a Voyager-DE (Applied Biosystems), an AXIMA (SHIMADZU BIOTECH), a ultraflex TOF/TOF (Bruker Daltonics), an Ettan MALDI-ToF Pro (amershambiosciences), and a prOTOF2000 (PerkinElmer).

All mass data detected by MALDI-TOF/MS may be corrected for the theoretical mass values of the mass standards. As a result of MALDI-TOF/MS, individual peaks may be processed by software to identify their corresponding peptides from databases. Moreover, the intensity of each detected peak is outputted as a numerical value and this value may be normalized to the internal standards. The numerical values thus obtained can be used as the measured levels of peptides corresponding to individual peaks.

If the amount of Aβ37 is increased in the presence of a candidate compound when compared to that in the presence of a vehicle (e.g., an aprotic polar solvent, preferably DMSO) alone, the candidate compound can be identified as a compound capable of enhancing Aβ37 production.

Alternatively, if the amount of Aβ40/42 is reduced and the amount of Aβ37 is increased in the presence of a candidate compound when compared to those in the presence of a vehicle (e.g., an aprotic polar solvent, preferably DMSO) alone, the candidate compound can be identified as a compound capable of inhibiting Aβ40/42 production and enhancing Aβ37 production.

Analysis of Aβ and Procedures for Analyzing Aggregation Ability of Aβ

As described above, the compound or its equivalent used in the present invention is capable of inhibiting Aβ aggregation. Thus, in this specification, the aggregation ability of Aβ in the presence of a candidate compound or the compound or its equivalent used in the present invention may be detected, determined or analyzed by the following procedures for Aβ analysis.

Aβ aggregation can be examined as changes in the circular dichroism (CD) spectrum at 215 to 260 nm induced by formation of β-sheet structure in Aβ. The CD spectrum at 215 to 260 nm is decreased when Aβ forms an α-helix or β-sheet structure. In particular, the formation of β-sheet structure is known to cause a decrease in the CD spectrum around 220 nm.

In another embodiment, for simple examination of Aβ aggregation in a solution, thioflavin T (ThT) may be used for fluorescence measurement of Aβ. An Aβ-containing solution may be supplemented with 1 to 100 μmol/L, preferably 1 to 20 μmol/L, and more preferably 10 μmol/L of ThT, and then immediately measured for fluorescence at an excitation wavelength of 450 nm and an emission wavelength of 490 nm (Wall J., Schell M., Murphy C., Hrncic R., Stevens F. J., Solomon A. (1999) Thermodynamic instability of human lambda 6 light chains: correlation with fibrillogenicity. Biochemistry. 38(42), 14101-14108). In these cases, the compound capable of inhibiting Aβ aggregation is a compound which prevents a decrease in the CD spectrum around 220 nm or which prevents an increase in the fluorescence intensity of ThT in the presence of ThT when compared to the absence of a candidate compound or the compound or its equivalent used in the present invention. In the identification or screening method of the present invention, a decrease in the aggregation ability of Aβ can be used as an index.

Procedures for Detecting Cell Toxicity of Aβ

When converted into a β-sheet structure, Aβ tends to form Aβ fiber aggregates and is known to show toxicity. As described above, the compound or its equivalent used in the present invention is capable of inhibiting Aβ aggregation. Namely, a candidate compound or the compound or its equivalent used in the present invention can inhibit the formation of β-sheet structure and hence can reduce the cell toxicity of Aβ. Thus, in the present invention, to detect a reduction in Aβ toxicity induced by a candidate compound or the compound or its equivalent used in the present invention, Aβ may be added to cells of the central nervous system, preferably brain-derived cells, preferably brain cortex-derived nerve cells, and more preferably brain cortex-derived primary cultured nerve cells, or glia cells such as astrocytes, or established cell lines such as PC12, followed by detection using known techniques for measuring cell damage (e.g., MTT assay, or cell damage assay using LDH level, alamar blue or trypan blue as an index). Aβ to be added to these cells may be either full-length Aβ or each Aβ (Aβ37, Aβ38, Aβ40 or Aβ42). Aβ of any length may be used as long as its sequence can form a β-sheet structure. Moreover, Aβ used for this purpose may be naturally-occurring, completely synthetic, or partially synthetic (i.e., partially derived from naturally-occurring Aβ). Naturally-occurring Aβ may be obtained in a manner known in the art. Each Aβ to be added to the cells may be, for example, dissolved in a 10 mM NaOH solution at 100 μg/ml and, after 5 minutes, diluted with phosphate buffered saline (PBS) to 500 μM.

MTT assay allows comparison and evaluation of cell survival activity by measuring cell toxicity in the above primary cultured nerve cells in the presence of each Aβ by using MTT and then calculating the ratio relative to the control group (Aβ-untreated group). For exarmple, a solution of thiazolyl blue tetrazolium bromide (MTT; SIGMA) is added at a final concentration of 0.4 to 0.8 mg/ml to the medium of primary cultured nerve cells grown for 1 to 3 days. After culturing at 37° C. for 20 minutes to 1 hour, the medium is removed, and the cells are solubilized in a vehicle (e.g., an aprotic polar solvent, preferably DMSO) and measured for their absorbance (550 nm). The medium used for this purpose is preferably Neurobasal/B27/2ME, by way of example.

The compound or its equivalent used in the present invention obtained by the identification or screening method of the present invention has an effect of enhancing Aβ37 production or an effect of inhibiting Aβ40/42 production and enhancing Aβ37 production; it is useful for treatment of Aβ-based diseases such as Alzheimer's disease and Down's syndrome.

7. Exogenous Aβ37 or Aβ38

(1) Aβ37 and Aβ38

The present invention provides an Aβ aggregation inhibitor and a nerve cell death inhibitor, each of which comprises at least one member selected from the group consisting of Aβ37, Aβ38, and their salts and hydrates thereof. Namely, the present invention provides an Aβ aggregation inhibitor and a nerve cell death inhibitor, each of which comprises Aβ37, Aβ38, a mutant thereof, a fragment thereof, a salt thereof, a hydrate thereof or a combination thereof (hereinafter also referred to as “the peptide or its equivalent used in the present invention”). In the peptide or its equivalent used in the present invention, Aβ37 is preferably a peptide containing the amino acid sequence shown in any one of SEQ ID NO: 12, 14 or 16, and more preferably a peptide consisting of the amino acid sequence shown in any one of SEQ ID NO: 12, 14 or 16. Likewise, in the peptide or its equivalent used in the present invention, Aβ38 is preferably a peptide containing the amino acid sequence shown in any one of SEQ ID NO: 18, 20 or 22, and more preferably a peptide consisting of the amino acid sequence shown in any one of SEQ ID NO: 18, 20 or 22. Aβ37 and Aβ38 may be in the form of a salt or a hydrate thereof, and these salt and hydrate forms are contemplated as being within the peptide or its equivalent used in the present invention.

| | |
|---|---|
| Human-type Aβ37 | |
| DAEFRHDSGYEVHHQKLVFFAEDVGSNKGAIIGLMV | (SEQ ID NO:12) |
| G | |
| Mouse-type Aβ37 | |
| DAEFGHDSGFEVRHQKLVFFAEDVGSNKGAIIGLMV | (SEQ ID NO:14) |
| G | |
| Rat-type Aβ37 | |
| DAEFGHDSGFEVRHQKLVFFAEDVGSNKGAIIGLMV | (SEQ ID NO:16) |
| G | |
| Human-type Aβ38 | |
| DAEFRHDSGYEVHHQKLVFFAEDVGSNKGAIIGLMV | (SEQ ID NO:18) |
| GG | |
| Mouse-type Aβ38 | |
| DAEFGHDSGFEVRHQKLVFFAEDVGSNKGAIIGLMV | (SEQ ID NO:20) |
| GG | |
| Rat-type Aβ38 | |
| DAEFGHDSGFEVRHQKLVFFAEDVGSNKGAIIGLMV | (SEQ ID NO:22) |
| GG | |

As used herein, the term “mutant” means a peptide containing substantially the same amino acid sequence as Aβ37 or Aβ38, preferably means a peptide containing substantially the same amino acid sequence as a peptide consisting of the amino acid sequence shown in any one of SEQ ID NO: 12, 14 or 16 or a peptide consisting of the amino acid sequence shown in any one of SEQ ID NO: 18, 20 or 22. Such a mutant is included in the peptide or its equivalent used in the present invention. Such a mutant may be in the form of a salt or a hydrate thereof, and these salt and hydrate forms are also contemplated as being within the mutant.

As used herein, the phrase “peptide containing substantially the same amino acid sequence” means a peptide which consists of an amino acid sequence derived from Aβ37 or Aβ38 (preferably a peptide consisting of the amino acid sequence shown in any one of SEQ ID NO: 12, 14 or 16, or a peptide consisting of the amino acid sequence shown in any one of SEQ ID NO: 18, 20 or 22) by deletion, substitution, insertion or addition, or a combination thereof, of one or more (preferably one or several) amino acids and which has an inhibitory activity against Aβ aggregation. The number of amino acids which may be deleted, substituted, inserted or added is, for example, 1 to 10, preferably 1 to 5, and particularly preferably 1 or 2.

As used herein, the term “substitution” means that one or more amino acid residues are replaced by other chemically equivalent amino acid residues without substantially altering the activity of a peptide. Examples include cases where one hydrophobic residue is replaced by another hydrophobic residue, where one polar residue is replaced by another polar residue having the same charge, etc. Functionally equivalent amino acids which allow these substitutions are known in the art for each amino acid. More specifically, examples of nonpolar (hydrophobic) amino acids include alanine, valine, isoleucine, leucine, proline, tryptophan, phenylalanine and methionine. Examples of polar (neutral) amino acids include glycine, serine, threonine, tyrosine, glutamine, asparagine and cysteine. Examples of positively-charged (basic) amino acids include arginine, histidine and lysine. Likewise, examples of negatively-charged (acidic) amino acids include aspartic acid and glutamic acid.

As used herein, the term “fragment” means a peptide which consists of a partial amino acid sequence of Aβ37 or Aβ38 and which has an inhibitory activity against Aβ aggregation. More specifically, the term “fragment” means a peptide which consists of a partial amino acid sequence of Aβ37 or Aβ38 (preferably a peptide containing the amino acid sequence shown in any one of SEQ ID NO: 12, 14 or 16 or a peptide containing the amino acid sequence shown in any one of SEQ ID NO: 18, 20 or 22, more preferably a peptide consisting of the amino acid sequence shown in any one of SEQ ID NO: 12, 14 or 16 or a peptide consisting of the amino acid sequence shown in any one of SEQ ID NO: 18, 20 or 22) or a mutant thereof and which has an inhibitory activity against Aβ aggregation. In this case, the number of amino acids which constitute such a peptide consisting of a partial amino acid sequence is, for example, 1 to 15, preferably 1 to 10, more preferably 1 to 7, and even more preferably 1 to 5. Such a fragment may be in the form of a salt or a hydrate thereof, and these salt and hydrate forms are also contemplated as being within the fragment. These fragments are included in the peptide or its equivalent used in the present invention.

The peptide or its equivalent used in the present invention further includes both those with and without a sugar chain(s). Thus, as long as these conditions are satisfied, the origin of the peptide or its equivalent used in the present invention is not limited to human, mouse or rat; peptides derived from non-human, non-mouse and non-rat mammals are also included.

The peptide or its equivalent used in the present invention may be obtained in various known manners. The peptide or its equivalent used in the present invention may be naturally-occurring or completely synthetic. Moreover, it may be partially synthetic, i.e., partially derived from naturally-occurring peptides. In the case of naturally-occurring peptides, cells from the living body may be cultured and then separated into cell and supernatant fractions in a known manner, e.g., by centrifugation or filtration, followed by collecting the supernatant fraction. Peptides or their equivalents contained in the culture supernatant may be purified by known separation and purification techniques, which are combined as appropriate. On the other hand, synthetic peptides may be synthesized according to routine techniques, such as liquid- and solid-phase techniques, usually using an automatic synthesizer. Chemically modified products of these peptides may be synthesized in a routine manner.

Alternatively, the peptide or its equivalent used in the present invention may be obtained using genetic engineering procedures and/or biochemical procedures. When using genetic engineering procedures and/or biochemical procedures, the peptide or its equivalent used in the present invention may be obtained by processing of APP through cleavage at the β- and γ-sites with β- and γ-secretases, respectively. More specifically, APP-expressing cells or cell membrane fragments thereof, which are prepared from the living body or APP transgenic non-human animals in a routine manner, may be treated with appropriate proteases, preferably β- and γ-secretases, to produce desired Aβ species. In this case, it is also possible to use the compound or its equivalent used in the present invention to ensure efficient production of the desired peptides or their equivalents mentioned above.

(2) Polynucleotides Encoding Aβ37 and Aβ38

According to another embodiment of the present invention, Aβ37 or Aβ38 may be prepared from a polynucleotide encoding the peptide or its equivalent used in the present invention, i.e., a polynucleotide encoding Aβ37 or Aβ38, a salt thereof, a hydrate thereof or a combination thereof. For example, a polynucleotide encoding the peptide or its equivalent used in the present invention may be introduced into appropriate host cells, and the resulting transformants may be cultured under conditions allowing expression of the polynucleotide, followed by separating and purifying the desired peptide from the culture by techniques commonly used for separation and purification of expressed proteins to prepare Aβ37 or Aβ38 (Sambrook and Russell, Molecular Cloning, 3rd edition, CSHL Press). Alternatively, a polynucleotide encoding the peptide or its equivalent used in the present invention may be applied to the so-called in vitro translation method based on a cell-free system using, e.g., rabbit reticulocyte lysate or E. coli lysate to prepare Aβ37 or Aβ38 (e.g., “Rapid Translation System” (Roche Applied Science), “Proteios” (TOYOBO)).

As used herein, the phrase “polynucleotide encoding Aβ37 or Aβ38” refers to a polynucleotide encoding the peptide or its equivalent used in the present invention, i.e., refers to a polynucleotide encoding:
preferably a peptide containing the amino acid sequence shown in any one of SEQ ID NO: 12, 14 or 16, or a peptide containing the amino acid sequence shown in any one of SEQ ID NO: 18, 20 or 22,
more preferably a peptide consisting of the amino acid sequence shown in any one of SEQ ID NO: 12, 14 or 16, or a peptide consisting of the amino acid sequence shown in any one of SEQ ID NO: 18, 20 or 22,
or a mutant thereof,
or a fragment thereof,
   or a salt of the polynucleotide or a hydrate thereof or a combination thereof (hereinafter also referred to as “the polynucleotide or its equivalent used in the present invention”). As described above, the polynucleotide or its equivalent used in the present invention may be in the form of a salt or a hydrate thereof, and these salt and hydrate forms are also contemplated as being within the polynucleotide or its equivalent used in the present invention.

Such a polynucleotide encoding a peptide consisting of the amino acid sequence shown in any one of SEQ ID NO: 12, 14 or 16 or a peptide consisting of the amino acid sequence shown in any one of SEQ ID NO: 18, 20 or 22 may preferably be a polynucleotide consisting of the nucleotide sequence shown in any one of SEQ ID NO: 11, 13 or 15, a polynucleotide consisting of the nucleotide sequence shown in any one of SEQ ID NO: 17, 19 or 21, or a homolog of the polynucleotide or a salt thereof or a hydrate thereof.

Likewise, a polynucleotide encoding a mutant included in the peptide or its equivalent used in the present invention may preferably be a homolog of a polynucleotide consisting of the nucleotide sequence shown in any one of SEQ ID NO: 11, 13 or 15, a homolog of a polynucleotide consisting of the nucleotide sequence shown in any one of SEQ ID NO: 17, 19 or 21, or a salt of the homolog or a hydrate thereof.

Likewise, a polynucleotide encoding a fragment included in the peptide or its equivalent used in the present invention may preferably be a part of a polynucleotide consisting of the nucleotide sequence shown in any one of SEQ ID NO: 11, 13 or 15, a part of a polynucleotide consisting of the nucleotide sequence shown in any one of SEQ ID NO: 17, 19 or 21, or a salt of the partial polynucleotide or a hydrate thereof.

As used herein, the term “polynucleotide” includes both DNA and RNA.

| | |
|---|---|
| Human-type Aβ37 | |
| GATGCAGAATTCCGACATGACTCAGGATATGAAGTT | (SEQ ID NO:11) |
| CATCATCAAAAATTGGTGTTCTTTGCAGAAGATGTG | |
| GGTTCAAACAAAGGTGCAATCATTGGACTCATGGTG | |
| GGC | |
| Mouse-type Aβ37 | |
| GATGCAGAATTCGGACATGATTCAGGATTTGAAGTC | (SEQ ID NO:13) |
| CGCCATCAAAAACTGGTGTTCTTTGCTGAAGATGTG | |
| GGTTCGAACAAAGGCGCCATCATCGGACTCATGGTG | |
| GGC | |
| Rat-type Aβ37 | |
| GATGCGGAGTTCGGACATGATTCAGGCTTCGAAGTC | (SEQ ID NO:15) |
| CGCCATCAAAAACTGGTGTTCTTTGCAGAAGATGTG | |
| GGTTCAAACAAAGGTGCCATCATTGGACTCATGGTG | |
| GGT | |
| Human-type Aβ38 | |
| GATGCAGAATTCCGACATGACTCAGGATATGAAGTT | (SEQ ID NO:17) |
| CATCATCAAAAATTGGTGTTCTTTGCAGAAGATGTG | |
| GGTTCAAACAAAGGTGCAATCATTGGACTCATGGTG | |
| GGCGGT | |
| Mouse-type Aβ38 | |
| GATGCAGAATTCGGACATGATTCAGGATTTGAAGTC | (SEQ ID NO:19) |
| CGCCATCAAAAACTGGTGTTCTTTGCTGAAGATGTG | |
| GGTTCGAACAAAGGCGCCATCATCGGACTCATGGTG | |
| GGCGGC | |
| Rat-type Aβ38 | |
| GATGCGGAGTTCGGACATGATTCAGGCTTCGAAGTC | (SEQ ID NO:21) |
| CGCCATCAAAAACTGGTGTTCTTTGCAGAAGATGTG | |
| GGTTCAAACAAAGGTGCCATCATTGGACTCATGGTG | |
| GGTGGC | |

As used herein, the term “homolog” means a polynucleotide that hybridizes to a polynucleotide encoding Aβ37 or Aβ38, preferably means a polynucleotide that hybridizes to a polynucleotide consisting of the nucleotide sequence shown in any one of SEQ ID NO: 11, 13 or 15 or a polynucleotide consisting of the nucleotide sequence shown in any one of SEQ ID NO: 17, 19 or 21. Such a homolog may be in the form of a salt or a hydrate thereof, and these salt and hydrate forms are also contemplated as being within the homolog. Such a homolog is included in the polynucleotide or its equivalent used in the present invention.

As used herein, the phrase “polynucleotide that hybridizes” means a polynucleotide which has a nucleotide sequence that hybridizes, under stringent conditions, to a nucleotide sequence complementary to a polynucleotide encoding Aβ37 or Aβ38 (preferably a nucleotide sequence complementary to a polynucleotide consisting of the nucleotide sequence shown in any one of SEQ ID NO: 11, 13 or 15 or a polynucleotide consisting of the nucleotide sequence shown in any one of SEQ ID NO: 17, 19 or 21) and which encodes a peptide having an inhibitory activity against Aβ aggregation. Examples of stringent conditions include “2×SSC, 0.1 % SDS, 50° C.”, “2×SSC, 0.1% SDS, 42° C.” and “1×SSC, 0.1% SDS, 37° C.”; and examples of more stringent conditions include “2×SSC, 0.1% SDS, 65° C.”, “0.5×SSC, 0.1% SDS, 42° C.” and “0.2×SSC, 0.1% SDS, 65° C.” More specifically, in the case of using Rapid-Hyb buffer (Amersham Life Science), the following conditions are considered: pre-hybridization at 68° C. for 30 minutes or longer, hybridization at 68° C. for 1 hour or longer in the presence of probes, followed by washing three times in 2×SSC, 0.1% SDS at room temperature for 20 minutes, three times in 1×SSC, 0.1% SDS at 37° C. for 20 minutes and finally twice in 1×SSC, 0.1% SDS at 50° C. for 20 minutes. Examples of a polynucleotide that hybridizes include those containing a nucleotide sequence sharing a homology of at least 50% or more, preferably 70%, more preferably 80%, even more preferably 90% (e.g., 95% or more) with a polynucleotide consisting of the nucleotide sequence shown in any one of SEQ ID NO: 11, 13, 15, 17, 19 or 21.

The GenBank accession numbers of the above amino acid and nucleotide sequences are as follows: NM_{—}000484 (nucleotide sequence) and NP_{—}000475 (amino acid sequence) for human Aβ37; NM_{—}007471 (nucleotide sequence) and NP_{—}031497 (amino acid sequence) for mouse Aβ37; NM_{—}019288 (nucleotide sequence) and NP_{—}062161 (amino acid sequence) for rat Aβ37; NM_{—}000484 (nucleotide sequence) and NP_{—}000475 (amino acid sequence) for human Aβ38; NM_{—}007471 (nucleotide sequence) and NP_{—}031497 (amino acid sequence) for mouse Aβ38; and NM_{—}019288 (nucleotide sequence) and NP_{—}062161 (amino acid sequence) for rat Aβ38.

The polynucleotide or its equivalent used in the present invention may be, for example, naturally-occurring or completely synthetic. Moreover, it may be partially synthetic, i.e., partially derived from naturally-occurring polynucleotides. Typical procedures for obtaining the polynucleotide or its equivalent used in the present invention involve screening from commercially available libraries or cDNA libraries through techniques commonly used in the art of genetic engineering, for example, by using appropriate DNA probes created on the basis of partial amino acid sequence information.

The peptide or its equivalent used in the present invention or a peptide encoded by the polynucleotide or its equivalent used in the present invention has an inhibitory effect against Aβ aggregation. Such an inhibitory effect against Aβ aggregation can be confirmed by the analysis procedures for the aggregation ability of Aβ described above in “6. Method for identifying or screening the compound or its equivalent used in the present invention.” Aβ aggregation has been found to cause cell death in nerve cells with Aβ deposition. Thus, the peptide or its equivalent used in the present invention or the polynucleotide or its equivalent used in the present invention which encodes the peptide may also be used as an Aβ aggregation inhibitor or a nerve cell death inhibitor.

For use as an Aβ aggregation inhibitor or a nerve cell death inhibitor, the polynucleotide or its equivalent of the present invention may be used alone or may be inserted into an appropriate vector or linked to an additional sequence such as a signal sequence or a polypeptide-stabilizing sequence.

For this purpose, known vectors may be used including adenovirus vector, retrovirus vector, plasmid, phagemid, and cosmid.

8. Pharmaceutical Compositions

The present invention provides a method for treating an Aβ-based disease. The above method may be accomplished by administering to a mammal in need of treatment of the disease, an effective amount of the compound or its equivalent used in the present invention, i.e., at least one member selected from the group consisting of a compound capable of enhancing Aβ37 production, a compound capable of inhibiting Aβ40/42 production and enhancing Aβ37 production, and salts of the compounds and hydrates thereof. The present invention includes a pharmaceutical composition containing, as an active ingredient, the compound or its equivalent used in the present invention, i.e., at least one member selected from the group consisting of a compound capable of enhancing Aβ37 production, a compound capable of inhibiting Aβ40/42 production and enhancing Aβ37 production, and salts of the compounds and hydrates thereof.

Alternatively, the method of the present invention for treating an Aβ-based disease may be accomplished by administering to a mammal in need of treatment of the disease, an effective amount of the peptide or its equivalent used in the present invention, i.e., Aβ37 or Aβ38, preferably a peptide containing the amino acid sequence shown in any one of SEQ ID NO: 12, 14 or 16 or a peptide containing the amino acid sequence shown in any one of SEQ ID NO: 18, 20 or 22, more preferably a peptide consisting of the amino acid sequence shown in any one of SEQ ID NO: 12, 14 or 16 or a peptide consisting of the amino acid sequence shown in any one of SEQ ID NO: 18, 20 or 22, or a mutant of the peptide or a fragment thereof, or a salt thereof or a hydrate thereof or a combination thereof.

Alternatively, the above method may be accomplished by administering to a mammal in need of treatment of the disease, an effective amount of the polynucleotide or its equivalent used in the present invention, i.e., a polynucleotide encoding Aβ37 or Aβ38, a salt thereof, a hydrate thereof or a combination thereof. Aβ37 or Aβ38 used for this purpose is preferably a peptide containing the amino acid sequence shown in any one of SEQ ID NO: 12, 14 or 16 or a peptide containing the amino acid sequence shown in any one of SEQ ID NO: 18, 20 or 22, more preferably a peptide consisting of the amino acid sequence shown in any one of SEQ ID NO: 12, 14 or 16 or a peptide consisting of the amino acid sequence shown in any one of SEQ ID NO: 18, 20 or 22, or a mutant of the peptide or a fragment thereof.

In the above method, the polynucleotide or its equivalent used in the present invention is more preferably a polynucleotide containing the nucleotide sequence shown in any one of SEQ ID NO: 11, 13 or 15 or a polynucleotide containing the nucleotide sequence shown in any one of SEQ ID NO: 17, 19 or 21, even more preferably a polynucleotide consisting of the nucleotide sequence shown in any one of SEQ ID NO: 11, 13 or 15 or a polynucleotide consisting of the nucleotide sequence shown in any one of SEQ ID NO: 17, 19 or 21, or a homolog of the polynucleotide, or a salt thereof or a hydrate thereof or a combination thereof, which may be administered in an effective amount.

The present invention includes a pharmaceutical composition containing, as an active ingredient, the peptide or its equivalent used in the present invention or the polynucleotide or its equivalent used in the present invention.

As used herein, the phrase “the pharmaceutical composition of the present invention” means a pharmaceutical composition containing, as an active ingredient, the compound or its equivalent used in the present invention, the peptide or its equivalent used in the present invention or the polynucleotide or its equivalent used in the present invention.

For use as an active ingredient, the compound or its equivalent used in the present invention, the peptide or its equivalent used in the present invention or the polynucleotide or its equivalent used in the present invention may be in the form of a prodrug.

As used herein, the term “prodrug” means an inactive form of “the active species of a drug” (that means a “drug” in relation to a prodrug), which is chemically modified with the aim of improving bioavailability, reducing side effects, etc. After being absorbed by the body, a prodrug will be metabolized into the active species and will exert its efficacy. Thus, the term “prodrug” refers to any compound, peptide or polynucleotide that has a lower intrinsic activity than the corresponding “drug,” but produces the “drug” substance when administered to a biological system, as a result of spontaneous chemical reactions, enzyme-catalyzed reactions or metabolic reactions. For the above purpose, various types of prodrugs may be exemplified, such as compounds, peptides and polynucleotides and their equivalents derived from those mentioned above by acylation, alkylation, phosphorylation, boration, carbonation, esterification, amidation or urethanization of amino, hydroxyl and/or carboxyl groups. However, these examples are only illustrative and not comprehensive. Those skilled in the art will be able to prepare various other known prodrugs in a known manner from the compounds, peptides, polynucleotides or their equivalents mentioned above. Prodrugs prepared from the compounds, peptides, polynucleotides or their equivalents mentioned above fall within the scope of the present invention.

As used herein, the term “Aβ-based disease” covers a wide variety of diseases including Alzheimer's disease (AD) (see, e.g., Documents 1, 2, 3, 4, 5, 6, 7 and 8); senile dementia of the Alzheimer's type (SDAT), senile dementia (see, e.g., Document 9); frontotemporal dementia (see, e.g., Document 10); Pick's disease (see, e.g., Document 11); Down's syndrome (see, e.g., Documents 12 and 13); cerebrovascular angiopathy (see, e.g., Documents 14, 15, 16 and 17); hereditary cerebral hemorrhage with amyloidosis (Dutch type) (see, e.g., Documents 18, 19, 20 and 21); cognitive impairment (see, e.g., Document 22); memory disorder, learning disability (see, e.g., Documents 23, 24 and 25); amyloidosis, cerebral ischemia (see, e.g., Documents 22, 26 and 27); cerebrovascular dementia (see, e.g., Document 28); ophthalmoplegia (see, e.g., Document 29); multiple sclerosis (see, e.g., Documents 30 and 31); head trauma (see, e.g., Document 32); apraxia (see, e.g., Document 33); prion disease, familial amyloid neuropathy, triplet repeat disease (see, e.g., Documents 34, 35 and 36); Parkinson's disease (see, e.g., Document 37), dementia with Lewy bodies (see, e.g., Documents 38, 39, 40 and 37); Parkinsonism-dementia complex (see, e.g., Documents 41 and 42); frontotemporal dementia-parkinsonism linked to chromosome 17 (see, e.g., Document 43); dementia with argyrophilic grains (see, e.g., Document 44); Niemann-Pick disease (see, e.g., Document 45); amyotrophic lateral sclerosis (see, e.g., Documents 46, 47, 48 and 49); hydrocephalus (see, e.g., Documents 50, 51, 52, 53 and 54); paraparesis (see, e.g., Documents 29, 33, 55 and 56); progressive supranuclear palsy (see, e.g., Documents 40 and 37); cerebral hemorrhage (see, e.g., Documents 57 and 58); convulsion (see, e.g., Document 59); mild cognitive impairment (see, e.g., Documents 60 and 61); and arteriosclerosis (see, e.g., Document 62).

(Document 1) Klein W L, et al., Alzheimer's disease-affected brain: Presence of oligomeric Aβ ligands (ADDLs) suggests a molecular basis for reversible memory loss, Proceedings of the National Academy of Sciences of the USA, Sep. 2, 2003, 100(18), p.10417-10422.

(Document 2) Nitsch R M, et al., Antibodies against β-amyloid slow cognitive decline in Alzheimer's disease, Neuron, May 22, 2003, 38(4), p.547-554.

(Document 3) Jarrett J T, et al., The carboxy terminus of the β amyloid protein is critical for the seeding of amyloid formation: Implications for the pathogenesis of Alzheimers' disease, Biochemistry, May 11, 1993, 32(18), p.4693-4697.

(Document 4) Glenner G G, et al., Alzheimer's disease; initial report of the purification and characterization of a novel cerebrovascular amyloid protein, Biochemical and biophysical research communications, May 16, 1984, 120(3), p.885-890.

(Document 5) Masters C L, et al., Amyloid plaque core protein in Alzheimer disease and Down syndrome, Proceedings of the National Academy of Sciences of the USA, June 1985, 82(12), p.4245-4249.

(Document 6) Gouras G K, et al., Intraneuronal Aβ42 accumulation in human brain, American journal of pathology, January 2000, 156(1), p.15-20.

(Document 7) Scheuner D, et al., Secreted amyloid β-protein similar to that in the senile plaques of Alzheimer's disease is increased in vivo by the presenilin 1 and 2 and APP mutations linked to familial Alzheimer's disease, Nature Medicine, August 1996, 2(8), p.864-870.

(Document 8) Forman M S, et al., Differential effects of the swedish mutant amyloid precursor protein on β-amyloid accumulation and secretion in neurons and nonneuronal cells, The journal of biological chemistry, Dec. 19, 1997, 272(51), p.32247-32253.

(Document 9) Blass J P, Brain metabolism and brain disease: Is metabolic deficiency the proximate cause of Alzheimer dementia? Journal of Neuroscience Research, Dec. 1, 2001, 66(5), p.851-856.

(Document 10) Evin G, et al., Alternative transcripts of presenilin-1 associated with frontotemporal dementia, Neuroreport, Apr. 16, 2002, 13(5), p.719-723.

(Document 11) Yasuhara O, et al., Accumulation of amyloid precursor protein in brain lesions of patients with Pick disease, Neuroscience Letters, Apr. 25, 1994, 171(1-2), p.63-66.

(Document 12) Teller J K, et al., Presence of soluble amyloid β-peptide precedes amyloid plaque formation in Down's syndrome, Nature Medicine, January 1996, 2(1), p.93-95.

(Document 13) Tokuda T, et al., Plasma levels of amyloid β proteins Aβ1-40 and Aβ1-42(43) are elevated in Down's syndrome, Annals of Neurology, February 1997, 41(2), p.271-273.

(Document 14) Hayashi Y, et al., Evidence for presenilin-1 involvement in amyloid angiopathy in the Alzheimer's disease-affected brain, Brain Research, Apr. 13, 1998, 789(2), p.307-314

(Document 15) Barelli H, et al., Characterization of new polyclonal antibodies specific for 40 and 42 amino acid-long amyloid β peptides: their use to examine the cell biology of presenilins and the immunohistochemistry of sporadic Alzheimer's disease and cerebral amyloid angiopathy cases, Molecular Medicine, October 1997, 3(10), p.695-707.

(Document 16) Calhoun M E, et al., Neuronal overexpression of mutant amyloid precursor protein results in prominent deposition of cerebrovascular amyloid, Proceedings of the National Academy of Sciences of the USA, Nov. 23, 1999, 96(24), p.14088-14093.

(Document 17) Dermaut B, et al., Cerebral amyloid angiopathy is a pathogenic lesion in Alzheimer's Disease due to a novel presenilin-1 mutation, Brain, December 2001, 124(12), p.2383-2392.

(Document 18) Cras P, et al., Presenile Alzheimer dementia characterized by amyloid angiopathy and large amyloid core type senile plaques in the APP 692Ala→Gly mutation, Acta Neuropathologica(Berl), September 1998, 96(3), p.253-260.

(Document 19) Herzig M C, et al., Aβ is targeted to the vasculature in a mouse model of hereditary cerebral hemorrhage with amyloidosis, Nature Neuroscience, September 2004, 7(9), p.954-960.

(Document 20) van Duinen S G, et al., Hereditary cerebral hemorrhage with amyloidosis in patients of Dutch origin is related to Alzheimer disease, Proceedings of the National Academy of Sciences of the USA, August 1987, 84(16), p.5991-5994

(Document 21) Levy E, et al., Mutation of the Alzheimer's disease amyloid gene in hereditary cerebral hemorrhage, Dutch type, Science, Jun. 1, 1990, 248(4959), p.1124-1126.

(Document 22) Laws S M, et al., Association between the presenilin-1 mutation Glu318Gly and complaints of memory impairment, Neurobiology of Aging, January-February 2002, 23(1), p.55-58.

(Document 23) Vaucher E, et al., Object recognition memory and cholinergic parameters in mice expressing human presenilin 1 transgenes, Experimental Neurology, June 2002, 175(2), p.398-406.

(Document 24) Morgan D, et al., Aβ peptide vaccination prevents memory loss in an animal model of Alzheimer's disease, Nature, Dec. 21-28, 2000, 408(6815), p.982-985.

(Document 25) Moran P M, et al., Age-related learning deficits in transgenic mice expressing the 751-amino acid isoform of human β-amyloid precursor protein, Proceedings of the National Academy of Sciences of the USA, Jun. 6, 1995, 92(12), p.5341-5345.

(Document 26) Koistinaho M, et al., β-amyloid precursor protein transgenic mice that harbor diffuse Aβ deposits but do not form plaques show increased ischemic vulnerability: Role of inflammation, Proceedings of the National Academy of Sciences of the USA, Feb. 5, 2002, 99(3), p.1610-1615.

(Document 27) Zhang F, et al., Increased susceptibility to ischemic brain damage in transgenic mice overexpressing the amyloid precursor protein, The journal of neuroscience, Oct. 15, 1997, 17(20), p.7655-7661.

(Document 28) Sadowski M, et al., Links between the pathology of Alzheimer's disease and vascular dementia, Neurochemical Research, June 2004, 29(6), p.1257-1266.

(Document 29) O'Riordan S, et al., Presenilin-1 mutation(E280G), spastic paraparesis, and cranial MRI white-matter abnormalities, Neurology, Oct. 8, 2002, 59(7), p.1108- 1110.

(Document 30) Gehrmann J, et al., Amyloid precursor protein (APP) expression in multiple sclerosis lesions, Glia, October 1995 15(2), p.141-51.

(Document 31) Reynolds W F, et al., Myeloperoxidase polymorphism is associated with gender specific risk for Alzheimer's disease, Experimental Neurology, January 1999, 155(1), p.31-41.

(Document 32) Smith D H, et al., Protein accumulation in traumatic brain injury, NeuroMolecular Medicine, 2003, 4(1-2), p.59-72.

(Document 33) Matsubara-Tsutsui M, et al., Molecular evidence of presenilin 1 mutation in familial early onset dementia, American journal of Medical Genetics, Apr. 8, 2002, 114(3), p.292-298.

(Document 34) Kirkitadze M D, et al., Paradigm shifts in Alzheimer's disease and other neurodegenerative disorders: the emerging role of oligomeric assemblies, Journal of Neuroscience Research, Sep. 1, 2002, 69(5), p.567-577.

(Document 35) Evert B O, et al., Inflammatory genes are upreglulated in expanded ataxin-3-expressing cell lines and spinocerebellar ataxia type 3 brains, The Journal of Neuroscience, Aug. 1, 2001, 21(15), p.5389-5396.

(Document 36) Mann D M, et al., Deposition of amyloid(A4) protein within the brains of persons with dementing disorders other than Alzheimer's disease and Down's syndrome, Neuroscience Letters, Feb. 5, 1990, 109(1-2), p.68-75.

(Document 37) Primavera J, et al., Brain accumulation of amyloid-β in Non-Alzheimer Neurodegeneration, Jornal of Alzheimer's Disease, October 1999, 1(3), p.183-193.

(Document 38) Giasson B I, et al., Interactions of amyloidogenic proteins. NeuroMolecular Medicine, 2003, 4(1-2), p.49-58.

(Document 39) Masliah E, et al., β-amyloid peptides enhance a-synuclein accumulation and neuronal deficits in a transgenic mouse model linking Alzheimer's disease and Parkinson's disease, Proceedings of the National Academy of Sciences of the USA, Oct. 9, 2001, 98(21), p.12245-12250.

(Document 40) Barrachina M, et al., Amyloid-β deposition in the cerebral cortex in Dementia with Lewy bodies is accompanied by a relative increase in AβPP mRNA isoforms containing the Kunitz protease inhibitor, Neurochemistry International, February 2005, 46(3), p.253-260.

(Document 41) Schmidt M L, et al., Amyloid plaques in Guam amyotrophic lateral sclerosis/parkinsonism-dementia complex contain species of Aβ similar to those found in the amyloid plaques of Alzheimer's disease and pathological aging, Acta Neuropathologica (Berl), February 1998, 95(2), p.117-122.

(Document 42) Ito H, et al., Demonstration of β amyloid protein-containing neurofibrillary tangles in parkinsonism-dementia complex on Guam, Neuropathology and applied neurobiology, October 1991, 17(5), p. 365-373.

(Document 43) Rosso S M, et al., Coexistent tau andamyloid pathology in hereditary frontotemporal dementia with tau mutations, Annals of the New York academy of sciences, 2000, 920, p.115-119.

(Document 44) Tolnay M, et al., Low amyloid(Aβ) plaque load and relative predominance of diffuse plaques distinguish argyrophilic grain disease from Alzheimer's disease, Neuropathology and applied neurobiology, August 1999, 25(4), p.295-305.

(Document 45) Jin L W, et al., Intracellular accumulation of amyloidogenic fragments of amyloid-β precursor protein in neurons with Niemann-Pick type C defects is associated with endosomal abnormalities, American Journal of Pathology, March 2004, 164(3), p.975-985.

(Document 46) Sasaki S, et al., Immunoreactivity of β-amyloid precursor protein in amyotrophic lateral sclerosis, Acta Neuropathologica(Berl), May 1999 97(5), p.463-468.

(Document 47) Tamaoka A, et al., Increased amyloid β protein in the skin of patients with amyotrophic lateral sclerosis, Journal of neurology, August 2000, 247(8), p.633-635.

(Document 48) Hamilton R L, et al., Alzheimer disease pathology in amyotrophic lateral sclerosis, Acta Neuropathologica, June 2004, 107(6), p.515-522.

(Document 49) Turner B J, et al., Brain β-amyloidaccumulation in transgenic mice expressing mutant superoxide dismutase 1, Neurochemical Research, December 2004, 29(12), p.2281-2286.

(Document 50) Weller R O, Pathology of cerebrospinal fluid and interstitial fluid of the CNS: Significance for Alzheimer disease, prion disorders and multiple sclerosis, Journal of Neuropathology and Experimental Neurology, October 1998, 57(10), p.885-894.

(Document 51) Silverberg G D, et al., Alzheimer's disease, normal-pressure hydrocephalus, and senescent changes in CSF circulatory physiology: a hypothesis, Lancet neurology, August 2003, 2(8), p.506-511.

(Document 52) Weller R O, et al., Cerebral amyloid angiopathy: Accumulation of Aβ in interstitial fluid drainage pathways in Alzheimer's disease, Annals of the New York academy of sciences, April 2000, 903, p.110-117.

(Document 53) Yow H Y, et al., A role for cerebrovascular disease in determining the pattern of β-amyloid deposition in Alzheimer's disease, Neurology and applied neurobiology, 2002, 28, p.149.

(Document 54) Weller R O, et al., Cerebrovascular disease is a major factor in the failure of elimination of Aβ from the aging human brain, Annals of the New York academy of sciences, November 2002, 977, p.162-168.

(Document 55) Smith M J, et al., Variable phenotype of Alzheimer's disease with spastic paraparesis, Annals of Neurology, 2001, 49(1), p.125-129.

(Document 56) Crook R, et al., A variant of Alzheimer's disease with spastic pararesis and unusual plaques due to deletion of exon 9 of presenilin 1, Nature Medicine, April 1998, 4(4), p.452-455.

(Document 57) Atwood C S, et al., Cerebrovascular requirement for sealant, anti-coagulant and remodeling molecules that allow for the maintenance of vascular integrity and blood supply, Brain Research Reviews, September 2003, 43(1), p.164-78.

(Document 58) Lowenson J D, et al., Protein aging: Extracellular amyloid formation and intracellular repair, Trends in cardiovascular medicine, 1994, 4(1), p.3-8.

(Document 59) Singleton A B, et al., Pathology of early-onset Alzheimer's disease cases bearing the Thr113-114ins presenilin-1 mutation, Brain, December 2000, 123(Pt12), p.2467-2474.

(Document 60) Gattaz W F, et al., Platelet phospholipase A2 activity in Alzheimer's disease and mild cognitive impairment, Journal of Neural Transmission, May 2004, 111(5), p.591-601.

(Document 61) Assini A, et al., Plasma levels of amyloid β-protein 42 are increased in women with mild cognitive impariment, Neurology, Sep. 14, 2004, 63(5), p.828-831.

(Document 62) De Meyer G R, et al., Platelet phagocytosis and processing of β-amyloid precursor protein as a mechanism of macrophage activation in atherosclerosis, Circulation Reserach, Jun. 14, 2002, 90(11), p.1197-1204.

As used herein, the term “treat”, “treating” or “treatment” generally means obtaining a desired pharmacological and/or physiological effect. The effect may be prophylactic in terms of completely or partially preventing a disease and/or symptom thereof, and may be therapeutic in terms of a partial or complete cure for an adverse effect attributable to a disease and/or symptom thereof. The term “treat”, “treating” or “treatment” as used herein covers any treatment of a disease in a mammal, particularly a human, and includes (a) to (c) shown below:
(a) preventing the disease or symptom from occurring in a patient who may be suspected to have a predisposition to the disease or symptom, but has not yet been diagnosed as having it;
(b) inhibiting the disease/symptom, i.e., arresting or slowing its progression; and
(c) relieving the disease/symptom, i.e., causing regression of the disease or symptom, or reversing the progression of the symptom.

The pharmaceutical composition of the present invention, preferably the therapeutic agent for an Aβ-based disease of the present invention, may be administered in various forms to a human or a non-human mammal either by oral route or by parenteral routes (e.g., intravenous injection, intramuscular injection, subcutaneous administration, intrarectal administration, percutaneous administration). Thus, a pharmaceutical composition containing the compound or its equivalent used in the present invention, the peptide or its equivalent used in the present invention or the polynucleotide or its equivalent used in the present invention may be used alone or may be formulated into an appropriate dosage form using pharmaceutically acceptable carriers in a manner commonly used depending on the route of administration.

Examples of preferred dosage forms include tablets, powders, fine granules, granules, coated tablets, capsules, syrups and troches for oral formulations, as well as inhalants, suppositories, injections (including drops), ointments, eye drops, ophthalmic ointments, nose drops, ear drops, poultices, lotions and liposomes for parenteral formulations.

As carriers used to formulate these formulations, for example, commonly-used excipients, binders, disintegrating agents, lubricants, coloring agents and correctives may be used, if necessary, in combination with stabilizing agents, emulsifiers, absorbefacients, detergents, pH adjustors, antiseptics, antioxidants, extenders, humectants, surface active agents, dispersants, buffers, preservatives, solvent aids, soothing agents, etc. In general, these formulations may be formulated in a routine manner by incorporating ingredients used as source materials for pharmaceutical formulations. Examples of such non-toxic ingredients available for use include animal and vegetable oils (e.g., soybean oil, beef tallow, synthetic glycerides); hydrocarbons (e.g., liquid paraffin, squalane, hard paraffin); ester oils (e.g., octyldodecyl myristate, isopropyl myristate); higher alcohols (e.g., cetostearyl alcohol, behenyl alcohol); silicon resins; silicone oil; detergents (e.g., polyoxyethylene fatty acid esters, sorbitan fatty acid esters, glycerine fatty acid esters, polyoxyethylene sorbitan fatty acid esters, polyoxyethylene hydrogenated castor oil, polyoxyethylene-polyoxypropylene block copolymers); water-soluble polymers (e.g., hydroxyethylcellulose, polyacrylic acid, carboxyvinyl polymers, polyethylene glycol, polyvinylpyrrolidone, methylcellulose); lower alcohols (e.g., ethanol, isopropanol); polyhydric alcohols (polyols) (e.g., glycerine, propylene glycol, dipropylene glycol, sorbitol, polyethylene glycol); saccharides (e.g., glucose, sucrose); inorganic powders (e.g., silicic acid anhydride, magnesium aluminum silicate, aluminum silicate); inorganic salts (e.g., sodium chloride, sodium phosphate); and purified water.

Examples of excipients include lactose, fructose, corn starch, sucrose, glucose, mannitol, sorbit, crystalline cellulose, and silicon dioxide. Examples of binders include polyvinyl alcohol, polyvinyl ether, methylcellulose, ethylcellulose, gum arabic, tragacanth, gelatin, shellac, hydroxypropylmethylcellulose, hydroxypropylcellulose, polyvinylpyrrolidone, polypropylene glycol-polyoxyethylene block polymers, and meglumine. Examples of disintegrating agents include starch, agar, powdered gelatin, crystalline cellulose, calcium carbonate, sodium hydrogen carbonate, calcium citrate, dextrin, pectin, and carboxymethyl cellulose calcium. Examples of lubricants include magnesium stearate, talc, polyethylene glycol, silica, and hydrogenated vegetable oils. Examples of coloring agents include those permitted for use in pharmaceutical preparations. Examples of correctives include cocoa powder, menthol, aromatic powder, peppermint oil, borneol, and cinnamon powder. The ingredients mentioned above may be in the form of a salt or a hydrate thereof.

In the case of oral formulations, for example, the compound or its equivalent used in the present invention, the peptide or its equivalent used in the present invention or the polynucleotide or its equivalent used in the present invention may be supplemented with excipients and, if necessary, with additional ingredients such as binders, disintegrating agents, lubricants, coloring agents and/or correctives, followed by formulation in a routine manner into powders, fme granules, granules, tablets, coated tablets, capsules, etc. Of course, tablets and granules may further be coated appropriately with sugar coating and the like, if necessary. In the case of, e.g., syrups and injectable formulations, for example, pH adjustors, solubilizers, isotonizing agents and the like may be incorporated, if necessary, in combination with solvent aids, stabilizing agents and the like, followed by formulation in a routine manner. In the case of external preparations, their manufacture is not limited in any way and they may be manufactured in a routine manner. As base ingredients used for external preparations, various types of materials commonly used in pharmaceutical preparations, quasi drugs, cosmetics and the like may be used, as exemplified by animal and vegetable oils, mineral oils, ester oils, waxes, higher alcohols, fatty acids, silicone oil, detergents, phospholipids, alcohols, polyhydric alcohols, water-soluble polymers, clay minerals, purified water, etc. If necessary, it is also possible to incorporate pH adjustors, antioxidants, chelating agents, antiseptic and antifungal agents, colorants, flavorings, etc. If necessary, it is further possible to incorporate other ingredients such as blood flow stimulators, disinfectants, antiphlogistics, cell-activating agents, vitamins, amino acids, moisturizers and keratolytic agents. In this case, the ratio of active ingredients to carriers may vary between 1% and 90% by weight. When used for the treatment mentioned above, the compound or its equivalent used in the present invention, the peptide or its equivalent used in the present invention or the polynucleotide or its equivalent used in the present invention is desirably purified to at least 90% or higher purity, preferably 95% or higher purity, more preferably 98% or higher purity, and even more preferably 99% or higher purity.

The peptide or its equivalent used in the present invention or the polynucleotide or its equivalent used in the present invention enables gene therapy in a patient with symptoms of an Aβ-based disease by administering an effective amount of the above polynucleotide or its equivalent to the patient in a routine manner and allowing the above peptide to be expressed in vivo. For example, the polynucleotide or its equivalent used in the present invention may be introduced into cells to cause expression of the above peptide in the cells, and these cells may then be transplanted into the patient to treat Aβ-based diseases. Alternatively, in a case where the polynucleotide or its equivalent used in the present invention is used for treatment, the polynucleotide or its equivalent may be used alone or may be linked to an additional sequence such as a signal sequence or a polypeptide-stabilizing sequence or inserted into an appropriate vector such as adenovirus vector or retrovirus vector, for administration to human or a non-human mammal in a routine manner. The polynucleotide or its equivalent used in the present invention may be administered as such or as a formulation together with pharmaceutically acceptable carriers in a routine manner through a catheter or a gene gun.

The above vector, into which the polynucleotide or its equivalent used in the present invention is inserted, may also be formulated in the same manner as described above and may be used, e.g., for parenteral purposes. Variations in dose level can be adjusted using standard empirical optimization procedures well understood in the art.

An effective dose of the pharmaceutical composition of the present invention containing the compound or its equivalent used in the present invention will vary, for example, depending on the severity of symptoms, age, sex, body weight, the intended dosage form, the type of salt, the actual type of disease, etc. In general, the daily dose for adults (body weight: 60 kg) is about 30 μg to 10 g, preferably 100 μg to 5 g, and more preferably 100 μg to 100 mg for oral administration, which may be given as a single dose or in divided doses, and about 30 μg to 1 g, preferably 100 μg to 500 mg, and more preferably 100 μg to 30 mg for injection administration, which may be given as a single dose or in divided doses.

The dosage form and the required dose range of the pharmaceutical composition of the present invention containing the peptide or its equivalent used in the present invention or the polynucleotide or its equivalent used in the present invention will depend on the choice of the peptide or its equivalent or the polynucleotide or its equivalent, a subject to be administered, the route of administration, formulation properties, the condition of a patient, and the doctor's judgment. However, the dose range preferred for appropriate administration is, for example, about 0.1 to 500 μg, preferably about 0.1 to 100 μg, and more preferably 1 to 50 μg per kg of patient's body weight. Taking into account that efficiency varies among administration routes, the required dose is expected to vary over a wide range. For example, oral administration is expected to require a higher dose than if administered by intravenous injection. Such variations in dose level can be adjusted using standard empirical optimization procedures well understood in the art.

9. Combination Therapy

(1) Embodiments

As used herein, the term “combination” includes cases where one of the components to be combined with each other is at least one member selected from the group consisting of the compound or its equivalent used in the present invention, the peptide or its equivalent used in the present invention, the polynucleotide or its equivalent used in the present invention and the pharmaceutical composition of the present invention, and the other component is a pharmaceutical composition containing at least one member selected from the group consisting of a ChE-inhibiting substance, an NMDA receptor antagonist and an AMPA receptor antagonist, or a pharmaceutical composition containing at least one member selected from the group consisting of an NMDA receptor antagonist and an AMPA receptor antagonist. With respect to the pharmaceutical composition of the present invention, i.e., the therapeutic agent for an Aβ-based disease, reference may be made to “8. Pharmaceutical compositions.”

In another embodiment of the present invention, such a combination is provided as a pharmaceutical composition (blended formulation) comprising at least one member selected from the group consisting of the compound or its equivalent used in the present invention, the peptide or its equivalent used in the present invention and the polynucleotide or its equivalent used in the present invention, as well as at least one member selected from the group consisting of a ChE-inhibiting substance, an NMDA receptor antagonist and an AMPA receptor antagonist, or at least one member selected from the group consisting of an NMDA receptor antagonist and an AMPA receptor antagonist.

In another embodiment of the present invention, the term “combination” includes cases where the components to be combined together are the compound or its equivalent used in the present invention and the peptide or its equivalent used in the present invention, or cases where the components to be combined together are the compound or its equivalent used in the present invention and the polynucleotide or its equivalent used in the present invention. Such a combination may be provided as a pharmaceutical composition (blended formulation) comprising the compound used in the present invention and the peptide or its equivalent used in the present invention or as a pharmaceutical composition (blended formulation) comprising the compound used in the present invention and the polynucleotide or its equivalent used in the present invention.

The present invention includes a method for treating an Aβ-based disease by combination therapy (hereinafter also referred to as “the combination therapy of the present invention”).

(2) Pharmaceutical Compositions (Blended Formulations)

(i) The present invention provides a pharmaceutical composition (blended formulation) comprising the compound or its equivalent used in the present invention, i.e., at least one member selected from the group consisting of a compound capable of enhancing Aβ37 production, a compound capable of inhibiting Aβ40/42 production and enhancing Aβ37 production, and salts of the compounds and hydrates thereof, as well as at least one member selected from the group consisting of a ChE-inhibiting substance, an NMDA receptor antagonist and an AMPA receptor antagonist.

(ii) The present invention provides a pharmaceutical composition comprising the peptide or its equivalent used in the present invention or the polynucleotide or its equivalent used in the present invention, i.e., Aβ37 or Aβ38, a mutant thereof, a fragment thereof, a salt thereof, a hydrate thereof or a combination thereof, or a polynucleotide encoding Aβ37 or Aβ38, a homolog thereof, a salt thereof, a hydrate thereof or a combination thereof, as well as at least one member selected from the group consisting of a ChE-inhibiting substance, an NMDA receptor antagonist and an AMPA receptor antagonist.

(iii) The present invention provides a pharmaceutical composition comprising the compound or its equivalent used in the present invention and the peptide or its equivalent used in the present invention.

(iv) The present invention provides a pharmaceutical composition comprising the compound or its equivalent used in the present invention and the polynucleotide or its equivalent used in the present invention.

As used herein, the phrase “pharmaceutical composition used in the combination therapy of the present invention” means the pharmaceutical compositions shown in (i) to (iv) above.

(3) ChE-Inhibiting Substances, NMDA Receptor Antagonists and AMPA Receptor Antagonists

A ChE-inhibiting substance, an NMDA receptor antagonist and an AMPA receptor antagonist are each used or developed as a therapeutic agent for an Aβ-based disease.

(i) ChE-Inhibiting Substances

A ChE-inhibiting substance in the context of the present invention refers to a compound having a ChE-inhibiting effect or its salt or hydrates thereof, which means a substance that reversibly or irreversibly inhibits ChE activity (i.e., ChE-inhibiting effect). In the present invention, ChE includes acetylcholinesterase (AChE) (EC3.1.1.7) and butyrylcholinesterase. ChE-inhibiting substances according to the present invention are preferably characterized by: having higher selectivity to AChE than to butyrylcholinesterase; having the ability to cross the blood-brain barrier; and not causing any sever side effect at a dose required for treatment, etc.

In the pharmaceutical composition used in the combination therapy of the present invention, a preferred compound to be combined or blended with at least one member selected from the group consisting of the compound or its equivalent used in the present invention, the peptide or its equivalent used in the present invention, the polynucleotide or its equivalent used in the present invention and the pharmaceutical composition of the present invention includes at least one member selected from the group consisting of a ChE-inhibiting substance and its salt and hydrates thereof, particularly at least one member selected from the group consisting of an AChE-inhibiting substance and its salt and hydrates thereof.

In the present invention, examples of ChE-inhibiting substances include donepezil (ARICEPT®), galanthamine (Reminyl®), tacrine (Cognex®), rivastigmine (Exelon®), zifrosilone (U.S. Pat. No. 5,693,668), physostigmine (Synapton), ipidacrine (U.S. Pat. No. 4,550,113), quilostigmine, metrifonate (Promem) (U.S. Pat. No. 4,950,658), eptastigmine, velnacrine, tolserine, cymserine (U.S. Pat. No. 6,410,747), mestinon, icopezil (U.S. Pat. No. 5,750,542), TAK-147 (J. Med. Chem., 37(15), 2292-2299, 1994, Japanese Patent No. 2650537, U.S. Pat. No. 5,273,974), huperzine A (Drugs Fut., 24, 647-663, 1999), stacofylline (U.S. Pat. No. 4,599,338), thiatolserine, neostigmine, eseroline or thiacymserine, 8-[3-[1-[(3-fluorophenyl)methyl]-4-piperidinyl]-1-oxopropyl]-1,2,5,6-tetrahydro-4H-pyrrolo[3,2,1-ij]quinolin-4-one (Japanese Patent No. 3512786), phenserine and ZT-1, or derivatives of the above compounds, or salts thereof or hydrates thereof, or prodrugs of the above compounds or derivatives, or salts thereof or hydrates thereof, or combinations thereof.

As a typical example, donepezil hydrochloride can be readily prepared as disclosed in, e.g., JP 01-79151 A, Japanese Patent No. 2578475, Japanese Patent No. 2733203, Japanese Patent No. 3078244 or U.S. Pat. No. 4,895,841. Galanthamine and its derivatives can be found in, e.g., U.S. Pat. No. 4,663,318, International Publication No. WO88/08708, International Publication No. WO97/03987, U.S. Pat. No. 6,316,439, U.S. Pat. No. 6,323,195 and U.S. Pat. No. 6,323,196. Tacrine and its derivatives can be found in, e.g., U.S. Pat. No. 4,631,286, U.S. Pat. No. 4,695,573, U.S. Pat. No. 4,754,050, International Publication No. WO88/02256, U.S. Pat. No. 4,835,275, U.S. Pat. No. 4,839,364, U.S. Pat. No. 4,999,430 and International Publication No. WO97/21681. Rivastigrnine and its derivatives can be found in, e.g., European Patent No. 193926, International Publication No. WO98/26775 and International Publication No. WO98/27055.

In the present invention, further examples of ChE-inhibiting substances include compounds having a ChE-inhibiting effect as described in International Publication No. WO00/18391.

For the above purpose, various types of prodrugs may be exemplified, such as compounds derived from those mentioned above by acylation, alkylation, phosphorylation, boration, carbonation, esterification, amidation or urethanization of amino, hydroxyl and/or carboxyl groups. However, these examples are only illustrative and not comprehensive. Those skilled in the art will be able to prepare various other known prodrugs in a known manner from the compounds mentioned above. Prodrugs prepared from the compounds mentioned above fall within the scope of the present invention.

(ii) NMDA Receptor Antagonists

An NMDA receptor antagonist in the context of the present invention means at least one member selected from the group consisting of a compound that binds to the NMDA receptor and inhibits its function, and a salt of the compound and hydrates thereof. NMDA receptor antagonists according to the present invention include memantine (3,5-dimethyl-adamantan-1-ylamine; CAS#19982-08-2), its derivatives or prodrugs thereof, or salts thereof (preferably hydrochloride salt) or hydrates thereof or combinations thereof. Memantine and derivatives thereof and their manufacturing method can be found in Japanese Patent No. 2821233.

(iii) AMPA Receptor Antagonists

An AMPA receptor antagonist in the context of the present invention means at least one member selected from the group consisting of a compound that binds to the AMPA receptor and inhibits its function, and a salt of the compound and hydrates thereof. AMPA receptor antagonists according to the present invention include talampanel (LY300164; (R)-(−)-1-(4-aminophenyl)-3-acetyl-4-methyl-7,8-methylenedioxy-3,4-dihydro-5H-2,3-benzodiazepine; CAS#161832-65-1), its derivatives or prodrugs thereof, or salts thereof or hydrates thereof or combinations thereof. Manufacturing method of talampanel can be found in J. Chem. Soc. Perkin Trans. I, 1995, p. 1423.

(4) Dosage Forms

When using at least one member selected from the group consisting of the compound or its equivalent used in the present invention, the peptide or its equivalent used in the present invention, the polynucleotide or its equivalent used in the present invention and the pharmaceutical composition of the present invention in combination with a pharmaceutical composition comprising at least one member selected from the group consisting of a ChE-inhibiting substance, an NMDA receptor antagonist and an AMPA receptor antagonist or at least one member selected from the group consisting of an NMDA receptor antagonist and an AMPA receptor antagonist, such a combination is useful in treating Aβ-based diseases. Likewise, a combination of the compound or its equivalent used in the present invention and the peptide or its equivalent used in the present invention, or a combination of the compound or its equivalent used in the present invention and the polynucleotide or its equivalent used in the present invention is also useful in treating Aβ-based diseases. Namely, the pharmaceutical composition used in the combination therapy of the present invention is useful in treating Aβ-based diseases.

In the combination therapy of the present invention, individual components to be combined may be given as such in effective amounts either at the same time or at certain intervals. Alternatively, in the form of separate pharmaceutical compositions formulated in a routine manner, individual components to be combined may be given in effective amounts either at the same time or at certain intervals. Alternatively, in the combination therapy of the present invention, individual components to be combined may be directly blended together into a formulation or may be partially pre-formulated and then blended together into a formulation. In this case, an effective amount of the resulting formulation may be given. Those skilled in the art will be able to formulate these components on the basis of commonly-used techniques (see “8. Pharmaceutical compositions” above).

There is no particular limitation on the dosage form of the pharmaceutical composition used in the combination therapy of the present invention; the pharmaceutical composition can be administered orally or parenterally (see “8. Pharmaceutical compositions” above). At the time of combination or blending, the individual components to be combined or blended may have different dosage forms or different doses.

The dose of the compound or its equivalent used in the present invention will vary, for example, depending on the severity of symptoms, age, sex, body weight, the intended dosage form, the type of salt, the actual type of disease, etc. In general, the daily dose for adults (body weight: 60 kg) is about 30 μg to 10 g, preferably 100 μg to 5 g, and more preferably 100 μg to 100 mg for oral administration and about 30 μg to 1 g, preferably 100 μg to 500 mg, and more preferably 100 μg to 30 mg for injection administration, which may be given as a single dose or in divided doses.

The dosage form and the required dose range of the peptide or its equivalent used in the present invention or the polynucleotide or its equivalent used in the present invention will depend on the choice of the peptide or its equivalent or the polynucleotide or its equivalent, a subject to be administered, the route of administration, formulation properties, the condition of a patient, and the doctor's judgment. However, the dose range preferred for appropriate administration is, for example, about 0.1 to 500 μg, preferably about 0.1 to 100 μg, and more preferably 1 to 50 μg per kg of patient's body weight. Taking into account that efficiency varies among administration routes, the required dose is expected to vary over a wide range. For example, oral administration is expected to require a higher dose than if administered by intravenous injection. Such variations in dose level can be adjusted using standard empirical optimization procedures well understood in the art.

With respect to oral dosage forms of ChE-inhibiting substances, fine granules of donepezil hydrochloride are available under the trade name ARICEPT fine granules (Eisai Co., Ltd.), while tablets of donepezil hydrochloride are available under the trade name ARICEPT tablets (Eisai Co., Ltd.). When administered in the form of a patch through percutaneous absorption, it is preferable to select a ChE-inhibiting substance which is not salt-forming, i.e., in a so-called free form.

The dose of the above-mentioned ChE-inhibiting substance for oral administration is 0.001 to 1000 mg/day, preferably 0.01 to 500 mg/day, and more preferably 0.1 to 300 mg/day, per 60 kg of body weight in adults. Taking donepezil hydrochloride as an example, the dose for oral administration is preferably 0.1 to 300 mg/day, more preferably 0.1 to 100 mg/day, and even more preferably 1.0 to 50 mg/day. Likewise, tacrine is desirably administered at a dose of 0.1 to 300 mg/day, preferably 40 to 120 mg/day, rivastigmine is desirably administered at a dose of 0.1 to 300 mg/day, preferably 3 to 12 mg/day, and galanthamine is desirably administered at a dose of 0.1 to 300 mg/day, preferably 16 to 32 mg/day.

The preferred dose of the above-mentioned ChE-inhibiting substance for parenteral administration is 5 to 50 mg/day, more preferably 10 to 20 mg/day, when administered in the form of a patch. On the other hand, injections may be prepared by dissolving or suspending the ChE-inhibiting substance in a pharmaceutically acceptable carrier such as physiological saline or commercially available injectable distilled water to give a concentration of 0.1 μg/ml of carrier to 10 mg/ml of carrier. The injections thus prepared may be administered to patients in need of treatment at a dose of 0.01 to 5.0 mg/day, more preferably 0.1 to 1.0 mg/day, once to three times a day.

The dosage form and dose of the NMDA receptor antagonist (e.g., memantine) or the AMPA receptor antagonist (e.g., talampanel) will depend on a subject to be administered, the route of administration, formulation properties, the condition of a patient, and the doctor's judgment. For example, although the therapeutic dose preferred for oral administration of memantine is about 5 to 35 mg/day per adult (body weight: 60 kg), memantine is sufficiently permitted for use in treatment at a dose of 100 to 500 mg/day. Likewise, talampanel may be used at a dose of about 20 to 70 mg, preferably about 20 to 50 mg per adult (body weight: 60 kg) twice to four times a day, preferably three times a day.

The doses of the above NMDA receptor antagonist and AMPA receptor antagonist are not limited to those mentioned above, and may vary depending on the type of compound to be administered or its salt or hydrates thereof, differences in efficiency among administration routes, etc. For example, oral administration is expected to require a higher dose than if administered by intravenous injection. Such variations in dose level can be adjusted using standard empirical optimization procedures well understood in the art.

Doses at the time of combination or blending may be appropriately selected among those mentioned above.

10. Kits

The present invention provides a kit comprising the compound or its equivalent used in the present invention, i.e., at least one member selected from the group consisting of a compound capable of enhancing Aβ37 production, a compound capable of inhibiting Aβ40/42 production and enhancing Aβ37 production, and salts of the compounds and hydrates thereof, as well as at least one member selected from the group consisting of the above-mentioned ChE-inhibiting substance, NMDA receptor antagonist and AMPA receptor antagonist. For example, these ChE-inhibiting substance, NMDA receptor antagonist and AMPA receptor antagonist may be donepezil hydrochloride, memantine and talampanel, respectively.

The kit of the present invention may be used for detecting or predicting the effectiveness of the pharmaceutical composition used in the combination therapy of the present invention. For example, the kit of the present invention comprising the same active ingredients as contained in the pharmaceutical composition may be used to analyze the inhibitory activity against Aβ aggregation or nerve cell death by the method of the present invention, thus enabling detection or prediction of the therapeutic effectiveness of the pharmaceutical composition. The kit of the present invention may further comprise additional elements required for detection or prediction of therapeutic effectiveness, including buffers, enzymes, substrates, experimental tools, instructions for use, etc.

Alternatively, the kit of the present invention may be used in a method for screening or identifying a compound suitable for a pharmaceutical composition for use in combination therapy. The kit of the present invention may comprise known compounds, e.g., donepezil hydrochloride, memantine and talampanel mentioned above, which may be used as standards for screening or identification. The kit of the present invention may further comprise additional elements required for screening or identification, including individual reagents, instructions for use, etc.

Alternatively, the kit of the present invention may be used in the combination therapy of the present invention, i.e., treatment of Aβ-based diseases by combination therapy. Namely, a kit comprising the pharmaceutical composition of the present invention and at least one member selected from the group consisting of a ChE-inhibiting substance, an NMDA receptor antagonist and an AMPA receptor antagonist may be used for combination therapy of Aβ-based diseases. Likewise, a kit comprising a pharmaceutical composition containing the compound or its equivalent used in the present invention and at least one member selected from the group consisting of a ChE-inhibiting substance, an NMDA receptor antagonist and an AMPA receptor antagonist may also be used for combination therapy of Aβ-based diseases. With respect to the dose, dosage form and the like required for the kit of the present invention when used for treatment of Aβ-based diseases, reference may be made to “9. Combination therapy” above. Such a kit may further comprise additional elements required for administration, including syringes, injection needles, solvents, catheters, instructions for use, etc.

Moreover, in another embodiment of the kit of the present invention, the compound or its equivalent used in the present invention in the above embodiments may be replaced by the peptide or its equivalent used in the present invention or the polynucleotide or its equivalent used in the present invention. Namely, a kit is provided which uses Aβ37 or Aβ38, a mutant thereof, a fragment thereof, a salt thereof, a hydrate thereof or a combination thereof, or a polynucleotide encoding Aβ37 or Aβ38, a homolog thereof, a salt thereof, a hydrate thereof or a combination thereof.

Likewise, in another embodiment of the kit of the present invention, a kit is provided which uses the peptide or its equivalent used in the present invention or the polynucleotide or its equivalent used in the present invention instead of at least one member selected from the group consisting of a ChE-inhibiting substance, an NMDA receptor antagonist and an AMPA receptor antagonist in the above embodiments.

### EXAMPLES

The present invention will be further described in more detail in the following Examples and Preparation Examples, which are not intended to limit the scope of the invention and are put forth so as to provide those skilled in the art with a complete disclosure. Also, these examples are not intended to mean or imply that the disclosed experiments are all or the only experiments actually performed. Although efforts have been made to ensure accuracy with respect to numbers used here (e.g., amounts, temperatures, concentrations, etc.), some experimental errors and deviations should be accounted for and these numbers may be varied without departing from the scope of the present invention.

### Example 1

### Circular Dichroism (CD) Analysis of Aβ

(1) Treatment of Aβwith Hexafluoroisopropanol (HFIP)

Human-type Aβ1-42 (Peptide Institute, Inc., Prod. # 4349-v), human-type Aβ1-40 (Peptide Institute, Inc., Prod. # 4307-v), human-type Aβ1-38 (SIGMA, A0189) or human-type Aβ1-37 (Peptide Institute, Inc., custom synthesized) was dissolved in HFIP (SIGMA, H8508) at 1 mg/mL and the resulting solution was shaken for 2 hours at 4° C. The solution was then dispensed in 10 to 30 μL aliquots into 500 μL polypropylene microtubes and stored at −80° C. until use.

(2) Washing of Quartz Cells

Quartz cells with optical path lengths of 1 mm (maximum volume: 500 μL) and 2 mm (maximum volume: 1 mL) (JASCO Corporation) were filled with a 2% sodium dodecyl sulfate solution and washed for 20 minutes in an ultrasonic cleaner. The solution in the quartz cells was then discarded and the cells were filled again with a 2% sodium dodecyl sulfate solution, followed by washing for 20 minutes in an ultrasonic cleaner. The solution in the cells was then discarded and the inside of the cells was washed with distilled water. Subsequently, the cells were filled with a saturated solution of sodium hydroxide and washed for 20 minutes in an ultrasonic cleaner. The solution in the cells was then discarded and the inside of the cells was washed sequentially with distilled water and methanol. Finally, the inside of the cells was washed with acetone and air-dried at room temperature.

(3) Redissolution and Incubation of Aβ

The Aβ solutions in HFIP were evaporated using a centrifugal evaporator (Tomy Seiko Co., Ltd., CC-180 and ST-10) to remove HFIP and then dissolved in a solution of 10 mM HEPES containing 0.9% NaCl. Each of the resulting Aβ solution was incubated at 37° C. and measured for their CD at different time points. Further, in order to examine the effect of Aβ1-37, Aβ1-38 or Aβ1-40 on the aggregation ability of Aβ1-42, Aβ1-42 and each Aβ were mixed at a ratio of 1:3 (5 μM: 15 μM) and the resulting mixtures were measured for their CD in the same manner as shown above.

(4) Measurement on Standard Solution

A cylindrical quartz cell with an optical path length of 10 mm (JASCO Corporation) was filled with a 0.06% aqueous solution of ammonium-d-10-camphorsulfonate (Katayama Chemical Industries Co., Ltd, Prod. #05-1251) and measured under the following conditions. The CD measuring instrument used was a JASCO J720WI (JASCO Corporation).
Measurement range: 350 to 220 nm
Data interval: 1 nm
Scanning speed: 50 nm/sec
Number of accumulations: 1
Response: 2 sec
Band width: 1.0 nm
Sensitivity: 200 meg

When the standard solution was confirmed to provide a measured curve of normal distribution-like shape with a maximum around 290 nm, the instrument was judged as correctly functioning.

(5) CD Measurement

The Aβ-containing solutions were injected into washed quartz cells with an optical path length of 1 or 2 mm and measured for their CD. Until measurement, the quartz cells were allowed to stand at 37° C., 100% humidity. The CD measurement was performed under the following conditions.
Measurement range: 260 to 190 nm
Data interval: 1 nm
Scanning speed: 50 nm/sec
Number of accumulations: 2
Response: 2 sec
Band width: 1.0 nm
Sensitivity: 100 meg
   (6) Results

(6A) Secondary Structure of each Aβ

CD was measured for each Aβ solution incubated at 37° C. at different time points. During the period from the initiation of the measurement until 1 day after dissolution, all Aβ fragments showed CD spectra indicative of random structures (FIGS. 1-1 to 1-5). However, from 2 days after dissolution, only Aβ1-42 was detected as showing a CD spectrum indicative of the formation of β-sheet structure (FIG. 1-6). When the CD measurement was further continued until 5 days after dissolution, Aβ1-42 showed CD spectra indicative that Aβ1-42 remained in a β-sheet structure (FIGS. 1-7 to 1-9). In contrast, the other Aβ fragments showed CD spectra indicative of random structures even at 5 days after dissolution (FIGS. 1-1 to 1-9). This suggests that Aβ1-37 is less likely to form a β-sheet structure than Aβ1-42. This property was also found in Aβ1-38 and Aβ1-40, as in the case of Aβ1-37.

(6B) Effect of other Aβs on β-Sheet Structure Formation in Aβ1-42

The effect of Aβ1-37, Aβ1-38 or Aβ1-40 on the aggregation ability of Aβ1-42 was examined by CD measurement on a 1:3 mixture of Aβ1-42 and each Aβ.

During the period from the initiation of the measurement until 8 hours, all Aβ fragments showed CD spectra indicative of random structures (FIGS. 2-1 to 2-5). From 1 day after initiation of incubation, only Aβ1-42+buffer was detected as showing a CD spectrum indicative of the formation of β-sheet structure (FIG. 2-6). When the CD measurement was further continued until 3 days after dissolution, Aβ1-42+buffer showed CD spectra indicative that Aβ1-42 remained in a β-sheet structure (FIGS. 2-7 and 2-8). Likewise, the sample mixed with Aβ1-40 was detected at 2 days after dissolution as showing a CD spectrum indicative of the formation of β-sheet structure (FIG. 2-7). In contrast, the sample mixed with Aβ1-37 was detected at 3 days after dissolution as showing a CD spectrum indicative of the formation of β-sheet structure (FIG. 2-8), suggesting that Aβ1-37 has an effect of slowing the rate of β-sheet structure formation in Aβ1-42. This effect was also found in Aβ1-38 (FIG. 2-8). These results suggest that the inhibitory effect of Aβ1-40 against the formation of β-sheet structure in Aβ1-42 is weaker than that of Aβ1-37 or Aβ1-38.

### Example 2

### Thioflavin T (ThT) Analysis for Aggregation Ability of Aβ

(1) Analysis for Aggregation Ability of Aβ

Each human-type Aβ (Aβ1-37, Aβ1-38, Aβ1-40 or Aβ1-42) prepared in the same manner as shown in Example 1 above was dissolved again respectively in a solution of 10 mM HEPES containing 0.9% NaCl at a final concentration of 10 mM and incubated in a CO₂ incubator at 37° C. for different times. After addition of ThT (SIGMA) at a final concentration of 10 μM, each sample was transferred to a 96-well black plate (Corning) and stirred for 10 seconds, followed by measuring the fluorescence intensity for each sample. Using a fluorospectrometer (LJL Biosystems), the fluorescence intensity at a wavelength of 490 nm was measured with an excitation light of 450 nm wavelength. Next, to examine the effect of Aβ1-37, Aβ1-38 or Aβ1-40 on the aggregation ability of Aβ1-42, Aβ1-42 and each Aβ were mixed at a ratio of 1:3 and the resulting mixtures were measured for the fluorescence intensity of ThT in the same manner as shown above at different time points.

(2) Results

(2A) β-Sheet Structure Formation in each Aβ

In Aβ1-42, the fluorescence intensity of ThT was increased with increasing incubation time (FIG. 3-1, solid square, ▪), whereas Aβ1-37 (solid circle, ●), Aβ1-38 (solid triangle, ▴) or Aβ1-40 (open square, □) showed no increase in the fluorescence intensity.

(2B) Effect of other Aβs on β-Sheet Structure Formation in Aβ1-42

When Aβ1-42 was mixed with Aβ3 1-37 (solid circle, ●), Aβ1-38 (solid triangle, ▪) or Aβ1-40 (open square, □) at a ratio of 1:3, the increase in the fluorescence intensity was inhibited as compared to Aβ1-42 alone (solid square, ▪) (FIG. 3-2). The degree of inhibition was greater in the presence of Aβ1-37 or Aβ1-38 than in the presence of Aβ1-40 (FIG. 3-3). These results were well correlated with the results of CD analysis for β-sheet structure.

### Example 3

### Nerve Cell Toxicity of Aβ

(1) Preparation of Primary Cultured Nerve Cells

Brain cortices were isolated from Wistar rats at 18 days of embryonic age (Charles River Japan) and provided for culture. More specifically, fetuses were aseptically extracted from pregnant rats under ether anesthesia. Brains were extracted from these fetuses and immersed in ice-cold L-15 medium (Invitrogen or SIGMA). Brain cortices were collected from the extracted brains under a stereoscopic microscope. Pieces of brain cortex thus collected were enzymatically treated in an enzyme solution containing 0.25% trypsin (Invitrogen) and 0.01% DNase (SIGMA) at 37° C. for 30 minutes to disperse cells. In this case, the enzymatic reaction was stopped by addition of inactivated horse serum. The resulting enzymatically treated solution was centrifuged at 1500 rotations/minute for 5 minutes to remove the supernatant, followed by addition of 5 to 10 ml medium to the resulting cell pellets. The medium used was Neurobasal medium (Invitrogen Corp. Cat #21103-049, Carlsbad, Calif. USA) supplemented with 2% B-27 supplement (Invitrogen Corp. Cat #17504-044, Carlsbad, Calif. USA), 25 μM 2-mercaptoethanol (2-ME, WAKO. Cat #139-06861, Osaka, Japan), 0.5 mM L-glutamine (Invitrogen Corp. Cat #25030-081, Carlsbad, Calif. USA) and Antibiotics-Antimycotics (Invitrogen Corp. Cat #15240-062, Carlsbad, Calif. USA) (Neurobasal/B27/2ME). After addition of the medium, the cell pellets were gently pipetted to disperse the cells again. The resulting cell dispersion was filtrated through a 40 μm nylon mesh (cell strainer, Becton Dickinson Labware) to remove cell aggregates, thereby obtaining a nerve cell suspension. This nerve cell suspension was diluted with the medium and seeded onto a 96-well plate (BIOCOAT®, Poly-D-lysine coated, Becton Dickinson Labware) at an initial cell density of 1.6×10⁵ cells/100 μl/well. After the seeded cells were cultured for 1 day in an incubator with 5% CO₂, 95% air at 37° C., the medium was entirely replaced by fresh Neurobasal/B27/2ME.

(2) Aβ Addition and MTT Assay

Each Aβ (Aβ1-37, Aβ1-40 or Aβ1-42) was dissolved in a 10 mM NaOH solution at 100 μg/ml and, after 5 minutes, diluted with phosphate buffered saline (PBS) to 500 μM. Each sample was incubated for 3 days in an incubator with 5% CO₂, 95% air at 37° C. At 5 days after initiation of culturing, the medium was replaced and each Aβ was added to the cells. After culturing for an additional 48 hours, the samples were measured for their toxicity by MTT assay. After removing the medium, fresh warmed medium was added in a volume of 100 μl/well, and an 8 mg/ml solution of thiazolyl blue tetrazolium bromide (MTT; SIGMA) in D-PBS (Dulbecco's PBS, SIGMA) was further added in a volume of 5 μl/well. The samples were incubated for 20 minutes in an incubator with 5% CO₂, 95% air at 37° C. After removing the medium, dimethyl sulfoxide (DMSO) was added in a volume of 100 μl/well to sufficiently dissolve the precipitated MTT formazan crystals, followed by measuring the absorbance at 550 nm. The ratio relative to the control group (Aβ-untreated group, CTRL) (% of CTRL) was calculated for each sample and used for comparison and evaluation of cell survival activity.
% of CTRL=(A550_sample)/(A550_CTRL)×100
(wherein A550_sample represents sample well absorbance at 550 nm and A550_CTRL represents control well absorbance at 550 nm)
(3) Results

Cellular MTT activity was measured at 48 hours after addition of each Aβ(Aβ1-37, Aβ1-40 or Aβ1-42), indicating that there was no difference between Aβ1-37 and the control group. Aβ1-40 showed about a 10% decrease in the activity, and Aβ1-42 treatment caused about a 25% decrease in MTT activity (FIG. 4). Each Aβ having a longer C-terminal end showed stronger cell toxicity. It has been believed that the cell toxicity of Aβ is related to its aggregation state (β-sheet structure content); this could also be confirmed by the results of this example. Namely, it was indicated that Aβ1-37 was less likely to form a β-sheet structure and hence had lower cell toxicity when compared to Aβ1-42.

### Example 4

### Compound A

Synthesis of (E)-N-biphenyl-3-ylmethyl-3-[3-methoxy-4-(4-methylimidazol-1-yl)phenyl]acrylamide (represented by the following formula) 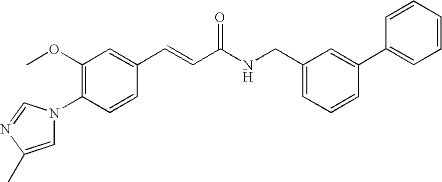
Synthesis of 3-methoxy-4-(4-methylimidazol-1-yl)benzaldehyde and 3-methoxy-4-(5-methylimidazol-1-yl)benzaldehyde

To a solution of 4-fluoro-3-methoxybenzaldehyde (3.00 g) and 4-methylimidazole (3.307 g) in N,N′-dimethylformamide (50 mL), potassium carbonate (4.05 g) was added, and the reaction mixture was stirred overnight at 100° C. The resulting reaction mixture was concentrated under reduced pressure, and the residue was added to water and ethyl acetate and partitioned between them to separate the organic layer. The organic layer was washed with saturated aqueous sodium chloride, dried over anhydrous magnesium sulfate and then concentrated under reduced pressure. The residue was purified by silica gel column chromatography (elution solvent: hexane-ethyl acetate system) to give 3-methoxy-4-(4-methylimidazol-1-yl)benzaldehyde (856 mg) and 3-methoxy-4-(5-methylimidazol-1-yl)benzaldehyde (44 mg).

The physical property data of 3-methoxy-4-(4-methylimidazol-1-yl)benzaldehyde are as shown below.

¹H-NMR (CDCl₃) δ (ppm): 2.31 (s,3H), 3.97 (s,3H), 7.02 (t,J=1.0 Hz,1H), 7.44 (d,J=8.0 Hz,1H), 7.55 (dd,J=1.6 Hz,8.0 Hz,1H), 7.58 (d,J=2.0 Hz,1H), 7.84 (d,J=1.6 Hz,1H), 10.00 (s,1H).

The physical property data of 3-methoxy-4-(5-methylimidazol-1-yl)benzaldehyde are as shown below.

¹H-NMR (CDCl₃) δ (ppm): 2.10 (s,3H), 3.90 (s,3H), 6.91 (t,J=1.0 Hz,1H), 7.40 (d,J=8.0 Hz,1H), 7.50 (d,J=1.2 Hz,1H), 7.57-7.59 (m,2H), 7.84 (s,1H), 10.05 (s,1H).

Synthesis of (E)-3-[3-methoxy-4-(4-methylimidazol-1-yl)phenyl]acrylic acid

To a solution of the thus obtained 3-methoxy-4-(4-methylimidazol-1-yl)benzaldehyde (4.00 g) in tetrahydrofuran (40 mL), diethylphosphonoacetic acid ethyl ester (4.00 mL) and lithium hydroxide monohydrate (932 mg) were added sequentially, and the reaction mixture was stirred overnight. After confirming the disappearance of the starting materials, 2N aqueous sodium hydroxide (30 mL) and ethanol (5 mL) were added to the reaction mixture, which was then stirred overnight at room temperature. The reaction mixture was cooled to 0° C., followed by addition of 2N hydrochloric acid (30 mL). The resulting precipitates were collected using a Kiriyama funnel and washed with water and ethyl acetate to give (E)-3-[3-methoxy-4-(4-methylimidazol-1-yl)phenyl]acrylic acid (4.61 g). The physical property data of the resulting compound are as shown below.

¹H-NMR (DMSO-d6) δ (ppm): 7.81 (s,1H), 7.60 (d,J=16 Hz,1H), 7.56 (s,1H), 7.39 (d,J=8.0 Hz,1H), 7.35 (d,J=8.0 Hz,1H), 7.16 (s,IH), 6.66 (d,J=16 Hz,1H), 3.88 (s,3H), 2.15 (s,3H).

Synthesis of (E)-N-biphenyl-3-ylmethyl-3-[3-methoxy-4-(4-methylimidazol-1-yl)phenyl]acrylamide

To a solution of (E)-3-[3-methoxy-4-(4-methylimidazol-1-yl)phenyl]acrylic acid (2.20 g) in N,N′-dimethylformamide (30 mL), 3-phenylbenzylamine hydrochloride (2.30 g) and diisopropylethylamine (4.57 mL) and 1-ethyl-3-(3-dimethylaminopropyl)carbodiimide hydrochloride (1.96 g) and 1-hydroxybenzotriazole (1.38 g) were added sequentially, and the reaction mixture was stirred overnight at room temperature. After confirming the disappearance of the starting materials, the reaction mixture was added to water and ethyl acetate and partitioned between them to separate the organic layer. The resulting organic layer was washed with saturated aqueous sodium chloride, dried over anhydrous magnesium sulfate and then concentrated under reduced pressure. The residue was purified by silica gel chromatography (elution solvent: ethyl acetate→ethyl acetate: ethanol=10:1) to give (E)-N-biphenyl-3-ylmethyl-3-[3-methoxy-4-(4-methylimidazol-1-yl)phenyl]acrylamide (3.30 g). The physical property data of the resulting compound are as shown below.

¹H-NMR (CDCl₃) δ (ppm): 7.71 (d,J=1.2 Hz,1H), 7.67 (d,J=16 Hz,1H), 7.52-7.60 (m,4H), 7.42-7.46 (m,3H), 7.37 (td,J=1.2,7.6 Hz,1H), 7.33 (brd,J=7.6 Hz,1H), 7.24 (d,J=8.0 Hz,1H), 7.17 (dd,J=1.6 Hz,6.4 Hz,1H), 7.13 (d,J=1.6 Hz,1H), 6.93 (t,J=1.2 Hz,1H), 6.45 (d,J=16 Hz,1H), 6.09 (brs,J=1H), 4.67 (d,J=5.6 Hz,2H), 3.87 (s,3H), 2.29 (s,3H).

### Example 5

### Compound B (CAS#501907-79-5)

Synthesis of N- {[(4-chlorophenyl)amino]iminomethyl}-N′-(4-cyanophenyl)urea (represented by the following formula) 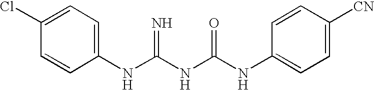
Synthesis of N-(4-chlorophenyl)guanidine p-toluenesulfonate salt

A solution of 4-chloroaniline (5.0 g) and cyanamide (1.91 g) and p-toluenesulfonic acid monohydrate (7.45 g) in toluene (60 mL) was heated under reflux for 12 hours. After the reaction mixture was allowed to cool to room temperature, ice-cold water (300 mL) was added to the reaction mixture, followed by stirring for 30 minutes. The solid matter precipitated in the reaction mixture was collected by suction filtration and air-dried overnight to give N-(4-chlorophenyl)guanidine p-toluenesulfonate salt (11.1 g). The physical property data of the resulting compound are as shown below.

¹H-NMR (DMSO-d₆) δ (ppm): 2.29 (s,3H), 7.12 (d,2H,J=8.0 Hz), 7.25 (d,2H,J=7.2 Hz), 7.31-7.62 (m,8H), 9.45-9.84 (brs,1H).

Synthesis of N-{[(4-chlorophenyl)amino]iminomethyl}-N′-(4-cyanophenyl)urea

To a solution of N-(4-chlorophenyl)guanidine p-toluenesulfonate salt (1.0 g) and 4-cyanophenyl isocyanate (422 mg) in acetone (30 mL), 5N aqueous sodium hydroxide (0.56 mL) was added, and the reaction mixture was stirred at room temperature for 4 hours. Subsequently, the reaction mixture was concentrated and the solid matter precipitated from the reaction mixture was collected by filtration. The resulting solid matter was washed with water (50 mL) and ethyl ether (50 mL) and then air-dried overnight to give N-{[(4-chlorophenyl)amino]iminomethyl}-N′-(4-cyanophenyl)urea (850 mg). The physical property data of the resulting compound were in agreement with the reported values (CAS #501907-79-54).

### Example 6

### Compound C (CAS#670250-40-5)

Synthesis of 5-{2-{3-[(1R)-1-hydroxymethyl-2-oxo-2-piperidin-1-ylethyl]ureido pyridin-4-yloxy}-1H-indole-1-carboxylic acid methylamide (represented by the following formula) 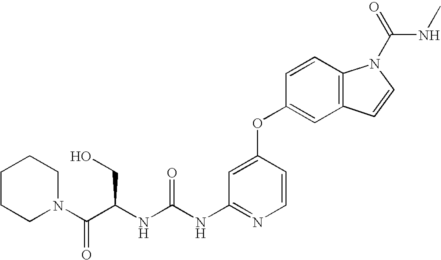
Synthesis of N1-methyl-5-(2-amino-4-pyridyl)oxy-1H-indolecarboxamide

To a DMF suspension of sodium hydride (containing 40% mineral oil, 430 mg), 4-(1H-5-indolyloxy)-2-pyridinamine (2.253 g, CAS #417722-11-3) described in International Publication No. WO02/32872 was slowly added under a nitrogen atmosphere at room temperature. The reaction mixture was stirred for 10 minutes at room temperature and then cooled in an ice-cold water bath, followed by addition of phenyl N-methylcarbamate (1.587 g). The reaction mixture was warmed to room temperature and stirred for 2 hours. The reaction mixture was added to ethyl acetate and water and partitioned between them to separate the organic layer. The resulting organic layer was washed sequentially with water and saturated aqueous sodium chloride, dried over anhydrous magnesium sulfate and then evaporated to remove the solvent. The residue was crystallized from ethyl acetate, and the precipitated crystals were collected by filtration and dried under ventilation to give N1-methyl-5-(2-amino-4-pyridyl)oxy-1H-indolecarboxamide (2.163 g) as a light brown crystal. The physical property data of the resulting compound are as shown below.

¹H-NMR (CDCl₃) δ (ppm): 3.09 (d,J=4.8 Hz,3H), 4.36 (m,2H), 5.49 (m,1H), 5.92 (d,1H,J=2.0 Hz), 6.30 (dd,J=6.0,2.0 Hz,1H), 6.61 (d,J=3.6 Hz,1H), 7.07 (dd,J=8.8,2.4 Hz,1H), 7.30 (d,J=2.4 Hz,1H), 7.45 (d,J=3.6 Hz,1H), 7.92 (d,J=6.0 Hz,1H), 8.17 (d,J=8.8 Hz,1H).

Synthesis of phenyl N-{4-[1-(methylamine)carbonyl-1H-5-indolyloxy]-2-pridyl}-N-(phenoxycarbonyl)carbamate

To a suspension of N1-methyl-5-(2-amino4-pyridyl)oxy-1H-indolecarboxamide (2.0 g) in THF (140 mL) and DMF (1.4 mL), triethylamine (2.2 mL) was added. Under ice cooling, phenyl chloroformate (1.8 mL) was added to this reaction mixture, which was then stirred at room temperature for 1.5 hours. After further addition of phenyl chloroformate (0.5 mL), this reaction mixture was stirred at room temperature for an additional 30 minutes. The reaction mixture was added to ethyl acetate and saturated aqueous sodium chloride and partitioned between them to separate the organic layer. The resulting organic layer was washed with saturated aqueous sodium chloride, dried over anhydrous magnesium sulfate and then evaporated to remove the solvent. Diethyl ether was added to the residue, and the precipitated crystals were collected by filtration, washed with diethyl ether and then dried under ventilation to give phenyl N-{4-[1-(methylamine)carbonyl-1H-5-indolyloxy]-2-pyridyl}-N-(phenoxycarbonyl)carbamate (3.3 g) as a light brown crystal. The physical property data of the resulting compound are as shown below.

¹H-NMR (DMSO-d₆) δ (ppm): 3.30 (d,J=4.4 Hz,3H), 6.66 (d,J=3.6 Hz,1H), 6.95 (dd,J=6.0,2.4 Hz,1H), 7.10 (dd,J=8.8,2.4 Hz,1H), 7.15-7.18 (m,4H), 7.27-7.31 (m,2H), 7.40-7.45 (m,5H), 7.52.(d,J=2.4 Hz,1H), 7.88 (d,J=3.6 Hz,1H), 8.17 (q,J=4.4 Hz,1H), 8.31 (d,J=8.8 Hz,1H), 8.41 (d,J=6.0Hz,1H).

Synthesis of 5-{2-3-[(1R)-1-hydroxymethyl-2-oxo-2-piperidin-1-ylethyl]ureido}pyridin-4-yloxy}-1H-indole-1-carboxylic acid methylamide

To a THF solution of (2R)-benziloxycarbonylamino-3-hydroxypropionic acid (1.91 g) and N-methylmorpholine (809 mg), isobutyl chloroformate (1.09 g) was added dropwise at −15° C. or below, and the reaction mixture was stirred for 30 minutes. After addition of pyrrolidine (1.13 g) at −15° C. or below, the reaction mixture was stirred at 0° C. for 30 minutes. The reaction mixture was added to ethyl acetate and water and partitioned between them to separate the organic layer. The resulting organic layer was washed sequentially with 1N hydrochloric acid, 1N aqueous sodium hydroxide, saturated aqueous sodium bicarbonate and saturated aqueous sodium chloride, dried over anhydrous magnesium sulfate and then evaporated to remove the solvent. The resulting residue was dissolved in methanol (15 mL) and THF (15 mL), followed by addition of 10% palladium-carbon (water-containing product, 300 mg). The reaction mixture was stirred at room temperature for 90 minutes under a hydrogen stream. After completion of the reaction, the reaction mixture was filtered through celite and the filtrate was concentrated under reduced pressure to give (2R)-amino-3-hydroxy-1-(piperidin-1-yl)propan-1-one (684 mg) as a colorless oil. To a solution of phenyl N-{4-[1-(methylamine)carbonyl-1H-5 -indolyloxy]-2-pyridyl}-N-(phenoxycarbonyl)carbamate (157 mg) and triethylamine (1.5 mL) in DMF (3 mL), (2R)-amino-3-hydroxy-1-(piperidin-1-yl)propan-1-one (228 mg) was added. The reaction mixture was stirred at room temperature for 18 hours and then added to ethyl acetate and saturated aqueous ammonium chloride and partitioned between them to separate the organic layer. The resulting organic layer was washed sequentially with water and saturated aqueous sodium chloride, dried over anhydrous magnesium sulfate and then evaporated to remove the solvent. The residue was purified by silica gel column chromatography (elution solvent: ethyl acetate:methanol=50: 1) and crystallized from a mixed solvent of ethyl acetate and hexane. The resulting crystals were collected by filtration and dried under ventilation to give 5-{2-{3-[(1R)-1-hydroxymethyl-2-oxo-2-piperidin-1-ylethyl]ureido}pyridin-4-yloxy}-1H-indole-1-carboxylic acid methylamide (107 mg) as a white crystal. The physical property data of the resulting compound are as shown below.

¹H-NMR (DMSO-d₆) δ (ppm): 1.36-1.61 (m,6H), 2.85 (d,J=4.4 Hz,3H), 3.40-3.53 (m,6H), 4.76 (m,1H), 4.92 (brs,1H), 6.54 (dd,J=6.0,2.4 Hz,1H), 6.69 (d,J=3.6 Hz,1H), 6.97 (d,J=2.4 Hz,1H), 7.06 (dd,J=9.0,2.4 Hz,lH), 7.38 (d,J=2.4 Hz,1H), 7.89 (d,J=3.6 Hz,1H), 8.05 (d,J=6.0 Hz,1H), 8.10-8.26 (m,2H), 8.30 (d,J=9.0 Hz,1H), 9.21 (s,1H).

### Example 7

### MALDI-TOF/MS Analysis of Aβ in Rat Fetal Brain-Derived Nerve Cell Culture Medium

(1) Rat Primary Nerve Cell Culturing

In the same manner as shown in Example 3 above, brain cortex-derived nerve cells were prepared from Wistar rats at 18 days of embryonic age. The nerve cell suspension was diluted with a medium and seeded onto 10 cm polystyrene culture dishes pre-coated with poly-D-lysine (BIOCOAT® cell environments Poly-D-lysine cell culture dish, Becton Dickinson Labware) in a volume of 15 ml/dish so as to give an initial cell density of 3.5×10⁵ cells/cm². After the seeded cells were cultured for 1 day in an incubator with 5% CO₂, 95% air at 37° C., the medium was entirely replaced by fresh Neurobasal/B27/2ME, followed by culturing for an additional 3 days.

(2) Addition of Compounds

At 4 days after initiation of culturing, the test compounds synthesized in the preceding Examples, i.e., Compound A, Compound B (CAS#501907-79-5) or Compound C (CAS#670250-40-5) were added as follows. The medium was entirely removed and replaced by Neurobasal/B27/2ME free from 2-ME (i.e., Neurobasal/B27) in a volume of 11 ml/dish. The test compounds (Compounds A, B and C) in DMSO were diluted with Neurobasal/B27 to 100-fold of their final concentration and added in a volume of 110 μl/dish, followed by sufficient mixing. The final DMSO concentration was kept at 1% or below. The control group received DMSO alone.

(3) Sampling

After addition of the test compounds, the cells were cultured for 3 days and the whole volume of the medium was collected from each dish. The resulting medium was provided as a MALDI-TOF/MS sample.

(4) Evaluation of Cell Survival

Cell survival was evaluated by MTT assay in the following manner. To the dishes after medium collection, warmed medium was added in a volume of 10 ml/dish and an 8 mg/ml MTT solution in D-PBS was added in a volume of 500 μl/dish. The dishes were incubated for 20 minutes in an incubator with 5% CO₂, 95% air at 37° C. After removing the medium, DMSO was added to the dishes in a volume of 10 ml/dish to sufficiently dissolve the precipitated MTT formazan crystals, followed by measuring the absorbance of each dish at 550 nm. The ratio relative to the control group (untreated group, CTRL) (% of CTRL) was calculated for each dish and used for comparison and evaluation of cell survival activity.

(5) Immunoprecipitation

Each sampled culture supernatant was collected in a 15 mL centrifuge tube and supplemented with 400 μL of a 25-fold concentrated solution of protease inhibitor cocktail Complete (Roche Diagnostics GmbH), followed by centrifugation at 4° C. at 3,000 rotations/minute for 5 minutes to sediment cell fragments. The resulting supernatant was transferred to another 15 mL centrifuge tube and supplemented with synthetic Aβ12-28 (Bachem) as an internal standard at a final concentration of 2 nM, followed by addition of 5 μg anti-Aβ monoclonal antibody (clone name: 4G8, Signet Laboratories, Inc). Subsequently, 5 μL of Protein G plus Protein A Agarose (Oncogene Research Products) was added after being blocked at 4° C. with 2% BSA and washed with TBS buffer. Further, 3-[(3-cholamidopropyl)dimethylammonio]-1-propanesulfonate (CHAPS; SIGMA) was added to each tube at a final concentration of 1%, followed by mixing at 4° C. for 4 to 8 hours.

(6) MALDI-TOF/MS [Matrix-Associated Laser Desorption Ionization-Time of Flight/Mass Spectrometry]

The Protein G plus Protein A Agarose holding the immunoprecipitated Aβ fragments adsorbed thereto was collected from each tube by centrifugation at 4° C. at 3,000 rotations/minute for 5 minutes and transferred to a 1.5 mL microtube. The Protein G plus Protein A Agarose was washed twice with 500 μL of 140 mM NaCl, 0.1% N-Octyl Glucoside (NOG; Loche Diagnostics GmbH), 10 mM Tris-HCl, pH 8, once with 500 μL of Tris-HCl, 10 mM, pH 8, and then once with 500 μL of ion exchanged water. After washing with ion exchanged water, as much fluid as possible was removed from each tube and Aβs were eluted with 5 μL of 0.2% NOG, 2.4% trifluoroacetic acid (TFA; PIERCE) and 48.7% acetonitrile (HPLC grade, Wako Pure Chemical Industries, Ltd.). Independently of this, α-cyano-4-hydroxy-cinnamic acid (CHCA; BRUKER DALTONICS) was dissolved in 0.2% NOG, 0.1% TFA and 33% acetonitrile at a saturating concentration and supplemented with human insulin (Peptide Institute, Inc.) and angiotensin III (Peptide Institute, Inc.) as mass standards at fmal concentrations of 167 nM and 56 nM, respectively, for use as a matrix solution. Each Aβ eluate (0.5 μL) and the matrix solution (0.5 μL) were spotted at the same position on a sample plate for mass spectrometry and air-dried at room temperature, followed by analysis with a mass spectrometer Voyager DE (Applied Biosystems). All mass data detected were corrected for the mass of human insulin and angiotensin III (5807.6 and 931.1, respectively). The normalization of the detected Aβ intensity between samples was performed assuming that the detected intensity of internal standard Aβ12-28 was the same in all samples.

Rat-type Aβ (SEQ ID NO: 16 and SEQ ID NO: 22) differs from human-type Aβ (SEQ ID NO: 12 and SEQ ID NO: 18) in amino acids at positions 5 (R→G), 10 (Y→F) and 13 (H→R) in the amino acid sequence, and is known to produce not only Aβ1-Y, but also Aβ11-Y as its major products (wherein Y is an integer of 32 to 42) (J. Neurochem. 71, 1920-1925, 1998).

On the other hand, among products from human-type APP, Aβ1-40 has been observed as the most major peak, while Aβ1-37, Aβ1-38 and Aβ1-42 have been observed as minor peaks (J. Biol. Chem. 271(50), 31894-31902, 1996). This pattern closely resembles that of rat primary cultured nerve cells observed in this example (provided that Aβ1-Y and Aβ11-Y are regarded as the same fragment) and, moreover, the sequence downstream of amino acid 14 is identical between rat-type and human-type. Namely, in relation to γ-site cleavage, findings obtained with rat-type Aβ can be adapted to human-type Aβ; this can be readily understood by those skilled in the art. Thus, data analysis in this example was performed on Aβ1-Y and Aβ11-Y (wherein Y is an integer of 32 to 42).

(7) Results

The results of matrix-associated laser desorption ionization-time of flight/mass spectrometry (MALDI-TOF/MS) analysis for each Aβ fragment in nerve cell culture supernatant in the absence of a test compound are as shown in FIG. 5-1, and FIG. 5-2 shows a magnified view of FIG. 5-1 in the molecular weight range between 2421 and 4565. For these results, the intensity of individual peaks is scored based on their height and area. Since the results of MALDI-TOF/MS analysis in nerve cell culture supernatant in the presence of a test compound were also obtained in the same format, peak area data were used as peak intensity values and normalized to the intensity of internal standard Aβ12-28 before being compared. FIGS. 6-1 to 6-3 show changes in the intensity of individual Aβ fragments examined at various concentrations of each test compound.

Compound A (FIG. 6-1)

Although no detectable change could be observed for Aβ1-42 or Aβ11-42, the figure indicated that Aβ1-40 or Aβ11-40 production was inhibited in a manner dependent on the concentration of Compound A. In contrast, Aβ1-37 or Aβ11-37 production and Aβ1-38 or Aβ11-38 production were found to be enhanced in a manner dependent on the concentration of Compound A.

Compound B (CAS#501907-79-5) (FIG. 6-2)

Although no detectable change could be observed for Aβ1-42 or Aβ11-42, the figure indicated that Aβ1-40 or Aβ11-40 production was inhibited in a manner dependent on the concentration of Compound B. In contrast, Aβ1-37 or Aβ11-37 production and Aβ1-38 or Aβ11-38 production were found to be enhanced in a manner dependent on the concentration of Compound B.

Compound C (CAS#670250-40-5) (FIG. 6-3)

Although no detectable change could be observed for Aβ1-42 or Aβ11-42, the figure indicated that Aβ1-40 or Aβ11-40 production tended to be inhibited. In contrast, Aβ1-37 or Aβ11-37 production and Aβ1-39 or Aβ11-39 production were found to be enhanced in a manner dependent on the concentration of Compound C.

### Example 8

### ELISA Quantification of Aβ in Rat Fetal Brain-Derived Nerve Cell Culture Medium

(1) Samples for ELISA Measurement

A part of each medium collected in Example 7(3) aforementioned was used as an ELISA sample. Each sample was not diluted for Aβ42 measurement, while it was diluted 5-fold for Aβ40 measurement with a diluent attached to an ELISA kit before being subjected to ELISA.

(2) Aβ ELISA

Aβ ELISA was performed using a Human Amyloid beta (1-42) Assay Kit (#17711, IBL Co., Ltd.) and a Human Amyloid beta (1-40) Assay Kit (#17713, IBL Co., Ltd.) in accordance with the kit's recommended protocol (the procedures described in the attached document), provided that a calibration curve for each Aβ was prepared using beta-amyloid peptide 1-42 (rat) or beta-amyloid peptide 1-40 (rat) (Calbiochem. #171596[Aβ_{4 2}] or #171593[Aβ_{4 0}]). The results were expressed as percentages (% of Control) relative to the Aβ concentration in the medium from the control group (untreated group, Control).

(3) Results

The results indicated that all of Compounds A, B and C inhibited Aβ40 (open square, □) and Aβ42 (solid square, ▪) production in a concentration-dependent manner (FIGS. 7-1 to 7-3).

The technical terms used herein are used only for the purpose of illustrating particular embodiments and are not intended for limiting purposes.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are as described above.

All publications mentioned herein are, for instance, incorporated by reference in their entirety for the purpose of describing and disclosing the cell lines, constructs, and methodologies which are reported in the publications used in connection with the invention described herein or are incorporated by reference for disclosure of the inventive methods for identifying and screening a compound as well as methods and compositions for use in these techniques; they can be used for practicing the present invention.

1: A method for inhibiting Aβ40 and Aβ42 production, which comprises using at least one member selected from the group consisting of a compound capable of enhancing Aβ37 production in the living body or a part thereof, and a salt of the compound and hydrates thereof to enhance Aβ37 production.

2: A method for inhibiting Aβ40 and Aβ42 production and enhancing Aβ37 production, which comprises using at least one member selected from the group consisting of a compound capable of inhibiting Aβ40 and Aβ42 production and enhancing Aβ37 production in the living body or a part thereof, and a salt of the compound and hydrates thereof.

3: A method for inhibiting Aβ aggregation, which comprises allowing Aβ37 and/or Aβ38 to act on Aβ42 in the living body or a part thereof.

4: A method for inhibiting Aβ aggregation, which comprises using at least one member selected from the group consisting of a compound capable of enhancing Aβ37 production in the living body or a part thereof, and a salt of the compound and hydrates thereof to enhance Aβ37 production.

5: A method for inhibiting Aβ aggregation, which comprises using at least one member selected from the group consisting of a compound capable of inhibiting Aβ40 and Aβ42 production and enhancing Aβ37 production in the living body or a part thereof, and a salt of the compound and hydrates thereof.

6: A method for preventing nerve cell death, which comprises allowing Aβ37 and/or Aβ38 to act on Aβ42 in the living body or a part thereof.

7: A method for preventing nerve cell death, which comprises using at least one member selected from the group consisting of a compound capable of enhancing Aβ37 production in the living body or a part thereof, and a salt of the compound and hydrates thereof to enhance Aβ37 production.

8: A method for preventing nerve cell death, which comprises using at least one member selected from the group consisting of a compound capable of inhibiting Aβ40 and Aβ42 production and enhancing Aβ37 production in the living body or a part thereof, and a salt of the compound and hydrates thereof.

9: The method according to claim 1, wherein the part of the living body is the brain.

10: An Aβ aggregation inhibitor which comprises at least one member selected from the group consisting of a compound capable of enhancing Aβ37 production, a compound capable of inhibiting Aβ40 and Aβ42 production and enhancing Aβ37 production, and salts of the compounds and hydrates thereof.

11: A nerve cell death inhibitor which comprises at least one member selected from the group consisting of a compound capable of enhancing Aβ37 production, a compound capable of inhibiting Aβ40 and Aβ42 production and enhancing Aβ37 production, and salts of the compounds and hydrates thereof.

12: A pharmaceutical composition which comprises at least one member selected from the group consisting of a compound capable of enhancing Aβ37 production, a compound capable of inhibiting Aβ40 and Aβ42 production and enhancing Aβ37 production, and salts of the compounds and hydrates thereof.

13: The pharmaceutical composition according to claim 12, which is used for treating an Aβ-based disease.

14: The pharmaceutical composition according to claim 13, wherein the Aβ-based disease is any one selected from the group consisting of Alzheimer's disease, senile dementia of the Alzheimer's type, mild cognitive impairment, senile dementia, Down's syndrome and amyloidosis.

15: An Aβ aggregation inhibitor which comprises at least one member selected from the group consisting of the following peptides (a) and (b), and fragments thereof:
(a) a peptide which contains the amino acid sequence shown in any one of SEQ ID NO: 12, SEQ ID NO: 14, SEQ ID NO: 16, SEQ ID NO: 18, SEQ ID NO: 20 and SEQ ID NO: 22; and
(b) a peptide which contains an amino acid sequence derived from the amino acid sequence shown in any one of SEQ ID NO: 12, SEQ ID NO: 14, SEQ ID NO: 16, SEQ ID NO: 18, SEQ ID NO: 20 and SEQ ID NO: 22 by deletion, substitution or addition, or a combination thereof, of one or several amino acids and which has an inhibitory activity against Aβ aggregation.

16: A nerve cell death inhibitor which comprises at least one member selected from the group consisting of the following peptides (a) and (b), and fragments thereof:
(a) a peptide which contains the amino acid sequence shown in any one of SEQ ID NO: 12, SEQ ID NO: 14, SEQ ID NO: 16, SEQ ID NO: 18, SEQ ID NO: 20 and SEQ ID NO: 22; and
(b) a peptide which contains an amino acid sequence derived from the amino acid sequence shown in any one of SEQ ID NO: 12, SEQ ID NO: 14, SEQ ID NO: 16, SEQ ID NO: 18, SEQ ID NO: 20 and SEQ ID NO: 22 by deletion, substitution or addition, or a combination thereof, of one or several amino acids and which has an inhibitory activity against Aβ aggregation.

17: A pharmaceutical composition which comprises at least one member selected from the group consisting of the following peptides (a) and (b), and fragments thereof:
(a) a peptide which contains the amino acid sequence shown in any one of SEQ ID NO: 12, SEQ ID NO: 14, SEQ ID NO: 16, SEQ ID NO: 18, SEQ ID NO: 20 and SEQ ID NO: 22; and
(b) a peptide which contains an amino acid sequence derived from the amino acid sequence shown in any one of SEQ ID NO: 12, SEQ ID NO: 14, SEQ ID NO: 16, SEQ ID NO: 18, SEQ ID NO: 20 and SEQ ID NO: 22 by deletion, substitution or addition, or a combination thereof, of one or several amino acids and which has an inhibitory activity against Aβ aggregation.

18: The pharmaceutical composition according to claim 17, which is used for treating an Aβ-based disease.

19: The pharmaceutical composition according to claim 18, wherein the Aβ-based disease is any one selected from the group consisting of Alzheimer's disease, senile dementia of the Alzheimer's type, mild cognitive impairment, senile dementia, Down's syndrome and amyloidosis.

20: An Aβ aggregation inhibitor which comprises a polynucleotide encoding at least one member selected from the group consisting of the following peptides (a) and (b), and fragments thereof:
(a) a peptide which contains the amino acid sequence shown in any one of SEQ ID NO: 12, SEQ ID NO: 14, SEQ ID NO: 16, SEQ ID NO: 18, SEQ ID NO: 20 and SEQ ID NO: 22; and
(b) a peptide which contains an amino acid sequence derived from the amino acid sequence shown in any one of SEQ ID NO: 12, SEQ ID NO: 14, SEQ ID NO: 16, SEQ ID NO: 18, SEQ ID NO: 20 and SEQ ID NO: 22 by deletion, substitution or addition, or a combination thereof, of one or several amino acids and which has an inhibitory activity against Aβ aggregation.

21: An Aβ aggregation inhibitor which comprises at least one member selected from the group consisting of the following polynucleotides (a) and (b):
(a) a polynucleotide which contains the nucleotide sequence shown in any one of SEQ ID NO: 11, SEQ ID NO: 13, SEQ ID NO: 15, SEQ ID NO: 17, SEQ ID NO: 19 and SEQ ID NO: 21; and
(b) a polynucleotide which hybridizes, under stringent conditions, to a polynucleotide consisting of a nucleotide sequence complementary to a polynucleotide consisting of the nucleotide sequence shown in any one of SEQ ID NO: 11, SEQ ID NO: 13, SEQ ID NO: 15, SEQ ID NO: 17, SEQ ID NO: 19 and SEQ ID NO: 21 and which encodes a peptide having an inhibitory activity against Aβ aggregation.

22: A nerve cell death inhibitor which comprises a polynucleotide encoding at least one member selected from the group consisting of the following peptides (a) and (b), and fragments thereof:
(a) a peptide which contains the amino acid sequence shown in any one of SEQ ID NO: 12, SEQ ID NO: 14, SEQ ID NO: 16, SEQ ID NO: 18, SEQ ID NO: 20 and SEQ ID NO: 22; and
(b) a peptide which contains an amino acid sequence derived from the amino acid sequence shown in any one of SEQ ID NO: 12, SEQ ID NO: 14, SEQ ID NO: 16, SEQ ID NO: 18, SEQ ID NO: 20 and SEQ ID NO: 22 by deletion, substitution or addition, or a combination thereof, of one or several amino acids and which has an inhibitory activity against Aβ aggregation.

23: A nerve cell death inhibitor which comprises at least one member selected from the group consisting of the following polynucleotides (a) and (b):
(a) a polynucleotide which contains the nucleotide sequence shown in any one of SEQ ID NO: 11, SEQ ID NO: 13, SEQ ID NO: 15,SEQ ID NO: 17, SEQ ID NO: 19 and SEQ ID NO: 21; and
(b) a polynucleotide which hybridizes, under stringent conditions, to a polynucleotide consisting of a nucleotide sequence complementary to a polynucleotide consisting of the nucleotide sequence shown in any one of SEQ ID NO: 11, SEQ ID NO: 13, SEQ ID NO: 15, SEQ ID NO: 17, SEQ ID NO: 19 and SEQ ID NO: 21 and which encodes a peptide having an inhibitory activity against Aβ aggregation.

24: A pharmaceutical composition which comprises a polynucleotide encoding at least one member selected from the group consisting of the following peptides (a) and (b), and fragments thereof:
(a) a peptide which contains the amino acid sequence shown in any one of SEQ ID NO: 12, SEQ ID NO: 14, SEQ ID NO: 16, SEQ ID NO: 18, SEQ ID NO: 20 and SEQ ID NO: 22; and
(b) a peptide which contains an amino acid sequence derived from the amino acid sequence shown in any one of SEQ ID NO: 12, SEQ ID NO: 14, SEQ ID NO: 16, SEQ ID NO: 18, SEQ ID NO: 20 and SEQ ID NO: 22 by deletion, substitution or addition, or a combination thereof, of one or several amino acids and which has an inhibitory activity against Aβ aggregation.

25: A pharmaceutical composition which comprises at least one member selected from the group consisting of the following polynucleotides (a) and (b):
(a) a polynucleotide which contains the nucleotide sequence shown in any one of SEQ ID NO: 11, SEQ ID NO: 13, SEQ ID NO: 15, SEQ ID NO: 17, SEQ ID NO: 19 and SEQ ID NO: 21; and
(b) a polynucleotide which hybridizes, under stringent conditions, to a polynucleotide consisting of a nucleotide sequence complementary to a polynucleotide consisting of the nucleotide sequence shown in any one of SEQ ID NO: 11, SEQ ID NO: 13, SEQ ID NO: 15, SEQ ID NO: 17, SEQ ID NO: 19 and SEQ ID NO: 21 and which encodes a peptide having an inhibitory activity against Aβ aggregation.

26: The pharmaceutical composition according to claim 24, which is used for treating an Aβ-based disease.

27: The pharmaceutical composition according to claim 26, wherein the Aβ-based disease is any one selected from the group consisting of Alzheimer's disease, senile dementia of the Alzheimer's type, mild cognitive impairment, senile dementia, Down's syndrome and amyloidosis.

28: A method for treating an Aβ-based disease, which comprises administering to a mammal in need of treatment of the disease, an effective amount of at least one member selected from the group consisting of a compound capable of enhancing Aβ37 production, a compound capable of inhibiting Aβ40 and Aβ42 production and enhancing Aβ37 production, and salts of the compounds and hydrates thereof.

29: A method for treating an Aβ-based disease, which comprises administering to a mammal in need of treatment of the disease, an effective amount of the pharmaceutical composition according to claim 12.

30: The method according to claim 28, wherein the Aβ-based disease is any one selected from the group consisting of Alzheimer's disease, senile dementia of the Alzheimer's type, mild cognitive impairment, senile dementia, Down's syndrome and amyloidosis.

31: The method according to claim 28, wherein the mammal is a human.

32: A method for identifying a compound capable of enhancing Aβ37 production, which comprises:
(a) contacting a candidate compound with a biological composition;
(b) measuring the amount of Aβ37 in the biological composition contacted with the candidate compound and the amount of Aβ37 in a biological composition not contacted with the candidate compound;
(c) selecting a candidate compound that produces an increase in the amount of Aβ37 in the biological composition contacted with the candidate compound when compared to the amount of Aβ37 in the biological composition not contacted with the candidate compound; and
(d) identifying the candidate compound obtained in (c) above as a compound capable of enhancing Aβ37 production.

33: A method for identifying a compound capable of inhibiting Aβ40 and Aβ42 production and enhancing Aβ37 production, which comprises:
(a) contacting a candidate compound with a biological composition;
(b) measuring the amounts of Aβ40, Aβ42 and Aβ37 in the biological composition contacted with the candidate compound and the amounts of Aβ40, Aβ42 and Aβ37 in a biological composition not contacted with the candidate compound;
(c) selecting a candidate compound that causes reductions in the amounts of Aβ40 and Aβ42 and also produces an increase in the amount of Aβ37 in the biological composition contacted with the candidate compound when compared to the amounts of Aβ40, Aβ42 and Aβ37 in the biological composition not contacted with the candidate compound; and
(d) identifying the candidate compound obtained in (c) above as a compound capable of inhibiting Aβ40 and Aβ42 production and enhancing Aβ37 production.

34: A method for screening a compound capable of enhancing Aβ37 production, which comprises:
(a) contacting a candidate compound with a biological composition;
(b) measuring the amount of Aβ37 in the biological composition contacted with the candidate compound and the amount of Aβ37 in a biological composition not contacted with the candidate compound;
(c) selecting a candidate compound that produces an increase in the amount of Aβ37 in the biological composition contacted with the candidate compound when compared to the amount of Aβ37 in the biological composition not contacted with the candidate compound; and
(d) identifying the candidate compound obtained in (c) above as a compound capable of enhancing Aβ37 production.

35: A method for screening a compound capable of inhibiting Aβ40 and Aβ42 production and enhancing Aβ37 production, which comprises:
(a) contacting a candidate compound with a biological composition;
(b) measuring the amounts of Aβ40, Aβ42 and Aβ37 in the biological composition contacted with the candidate compound and the amounts of Aβ40, Aβ42 and Aβ37 in a biological composition not contacted with the candidate compound;
(c) selecting a candidate compound that causes reductions in the amounts of Aβ40 and Aβ42 and also produces an increase in the amount of Aβ37 in the biological composition contacted with the candidate compound when compared to the amounts of Aβ40, Aβ42 and Aβ37 in the biological composition not contacted with the candidate compound; and
(d) identifying the candidate compound obtained in (c) above as a compound capable of inhibiting Aβ40 and Aβ42 production and enhancing Aβ37 production.

36: The method according to claim 32, wherein the biological composition comprises β-amyloid precursor protein-expressing cells.

37: The method according to claim 32, wherein the biological composition comprises mammalian cells.

38: The method according to claim 32, wherein the biological composition comprises nerve cells.

39: A pharmaceutical composition which comprises at least one member selected from the group consisting of a compound capable of enhancing Aβ37 production, a compound capable of inhibiting Aβ40 and Aβ42 production and enhancing Aβ37 production, and salts of the compounds and hydrates thereof, as well as at least one member selected from the group consisting of a cholinesterase-inhibiting substance, an NMDA receptor antagonist and an AMPA receptor antagonist.

40: The pharmaceutical composition according to claim 39, wherein the cholinesterase-inhibiting substance is donepezil or a salt thereof.

41: The pharmaceutical composition according to claim 39, wherein the NMDA receptor antagonist is memantine.

42: The pharmaceutical composition according to claim 39, wherein the AMPA receptor antagonist is talampanel.

43: The pharmaceutical composition according to claim 39, which is a therapeutic agent for an Aβ-based disease.

44: The pharmaceutical composition according to claim 43, wherein the Aβ-based disease is any one selected from the group consisting of Alzheimer's disease, senile dementia of the Alzheimer's type, mild cognitive impairment, senile dementia, Down's syndrome and amyloidosis.

45: A method for treating an Aβ-based disease, which comprises administering to a mammal in need of treatment of the disease, an effective amount of at least one member selected from the group consisting of a compound capable of enhancing Aβ37 production, a compound capable of inhibiting Aβ40 and Aβ42 production and enhancing Aβ37 production, and salts of the compounds and hydrates thereof, as well as an effective amount of at least one member selected from the group consisting of a cholinesterase-inhibiting substance, an NMDA receptor antagonist and an AMPA receptor antagonist.

46: The method according to claim 45, wherein the cholinesterase-inhibiting substance is donepezil or a salt thereof.

47: The method according to claim 45, wherein the NMDA receptor antagonist is memantine.

48: The method according to claim 45, wherein the AMPA receptor antagonist is talampanel.

49: The method according to claim 45, which is a therapeutic agent for an Aβ-based disease.

50: The method according to claim 49, wherein the Aβ-based disease is any one selected from the group consisting of Alzheimer's disease, senile dementia of the Alzheimer's type, mild cognitive impairment, senile dementia, Down's syndrome and amyloidosis.

51: The method according to claim 45, wherein the mammal is a human.

52: A kit which comprises at least one member selected from the group consisting of a compound capable of enhancing Aβ37 production, a compound capable of inhibiting Aβ40 and Aβ42 production and enhancing Aβ37 production, and salts of the compounds and hydrates thereof, as well as at least one member selected from the group consisting of a cholinesterase-inhibiting substance, an NMDA receptor antagonist and an AMPA receptor antagonist.

53: The kit according to claim 52, wherein the cholinesterase-inhibiting substance is donepezil or a salt thereof.

54: The kit according to claim 52, wherein the NMDA receptor antagonist is memantine.

55: The kit according to claim 52, wherein the AMPA receptor antagonist is talampanel.

56: The inhibitor according to claim 15, wherein the peptides (a) and (b) and fragments thereof are in the form of a salt or a hydrate thereof.

57: The inhibitor according to claim 16, wherein the peptides (a) and (b) and fragments thereof are in the form of a salt or a hydrate thereof.

58: The pharmaceutical composition according to claim 17, wherein the peptides (a) and (b) and fragments thereof are in the form of a salt or a hydrate thereof.

59: The inhibitor according to claim 20, wherein the polynucleotide(s) is/are in the form of a salt or a hydrate thereof.

60: The inhibitor according to claim 22, wherein the polynucleotide(s) is/are in the form of a salt or a hydrate thereof.

61: The pharmaceutical composition according to claim 24, wherein the polynucleotide(s) is/are in the form of a salt or a hydrate thereof.

62: The method according to claim 2, wherein the part of the living body is the brain.

63: The method according to claim 3, wherein the part of the living body is the brain.

64: The method according to claim 4, wherein the part of the living body is the brain.

65: The method according to claim 5, wherein the part of the living body is the brain.

66: The method according to claim 6, wherein the part of the living body, is the brain.

67: The method according to claim 7, wherein the part of the living body is the brain.

68: The method according to claim 8, wherein the part of the living body is the brain.

69: The pharmaceutical composition according to claim 25, which is used for treating an Aβ-based disease.

70: A method for treating an Aβ-based disease, which comprises administering to a mammal in need of treatment of the disease, an effective amount of the pharmaceutical composition according to claim 17.

71: A method for treating an Aβ-based disease, which comprises administering to a mammal in need of treatment of the disease, an effective amount of the pharmaceutical composition according to claim 24.

72: A method for treating an Aβ-based disease, which comprises administering to a mammal in need of treatment of the disease, an effective amount of the pharmaceutical composition according to claim 25.

73: The method according to claim 29, wherein the Aβ-based disease is any one selected from the group consisting of Alzheimer's disease, senile dementia of the Alzheimer's type, mild cognitive impairment, senile dementia, Down's syndrome and amyloidosis.

74: The method according to claim 29, wherein the mammal is a human.

75: The method according to claim 33, wherein the biological composition comprises β-amyloid precursor protein-expressing cells.

76: The method according to claim 34, wherein the biological composition comprises β-amyloid precursor protein-expressing cells.

77: The method according to claim 35, wherein the biological composition comprises β-amyloid precursor protein-expressing cells.

78: The method according to claim 33, wherein the biological composition comprises mammalian cells.

79: The method according to claim 34, wherein the biological composition comprises mammalian cells.

80: The method according to claim 35, wherein the biological composition comprises mammalian cells.

81: The method according to claim 33, wherein the biological composition comprises nerve cells.

82: The method according to claim 34, wherein the biological composition comprises nerve cells.

83: The method according to claim 35, wherein the biological composition comprises nerve cells.

84: The inhibitor according to claim 21, wherein the polynucleotide(s) is/are in the form of a salt or a hydrate thereof.

85: The inhibitor according to claim 23, wherein the polynucleotide(s) is/are in the form of a salt or a hydrate thereof.

86: The pharmaceutical composition according to claim 25, wherein the polynucleotide(s) is/are in the form of a salt or a hydrate thereof.


SEQUENCE LISTING (22 sequences)

SEQ ID NO: 1 - DNA, 2088 bp, Homo sapiens
  CDS (1)..(2085)
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 2 - protein, 695 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 3 - DNA, 2256 bp, Homo sapiens
  CDS (1)..(2256)
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccattcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 4 - protein, 751 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAIPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 5 - DNA, 2313 bp, Homo sapiens
  CDS (1)..(2313)
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 6 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 7 - DNA, 2088 bp, Mus musculus
  CDS (1)..(2088)
atgctgcccagcttggcactgctcctgctggccgcctggacggttcgggctctggaggtacccactgatggcaacgccgggctgctggcagaaccccagatcgccatgttctgtggtaaactcaacatgcacatgaatgtgcagaatggaaagtgggagtcagacccgtcagggaccaaaacctgcattggcaccaaggagggcatcttgcagtactgccaagaggtctaccctgaactgcagatcacaaacgtggtggaagccaaccagccagtgaccatccagaactggtgcaagcggggccgcaagcagtgcaagacacacacccacatcgtgattccttaccgttgcctagttggtgagtttgtgagcgacgcccttctcgtgcccgacaagtgcaagttcctacaccaggagcggatggatgtttgtgagacccatcttcactggcacaccgtcgccaaagagacatgcagcgagaagagcactaacttgcacgactatggcatgctgctgccctgcggcatcgacaagttccgaggggtagagtttgtatgctgcccgttggccgaggaaagcgacagcgtggattctgcggatgcagaggaggatgactctgatgtctggtggggtggagcggacacagactacgctgatggcggtgaagacaaagtagtagaagtcgccgaagaggaggaagtggctgatgttgaggaagaggaagctgatgatgatgaggatgtggaggatggggacgaggtggaggaggaggccgaggagccctacgaagaggccaccgagagaacaaccagcactgccaccaccaccacaaccaccactgagtccgtggaggaggtggtccgagttcccacgacagcagccagcacccccgacgccgtcgacaagtacctggagacacccggggacgagaacgagcatgcccatttccagaaagccaaagagaggctggaagccaagcaccgagagagaatgtcccaggtcatgagagaatgggaagaggcagagcgtcaagccaagaacttgcccaaagctgacaagaaggccgttatccagcatttccaggagaaagtggaatctctggaacaggaagcagccaatgagagacagcagcttgtagagacacacatggccagagttgaagccatgctcaatgaccgccgccgcctggccctcgagaattacatcactgcactgcaggcggtgcccccaaggcctcatcatgtgttcaacatgctgaagaagtacgtccgtgcggagcagaaagacagacagcacaccctaaagcattttgaacatgtgcgcatggtggaccccaagaaagctgctcagatccggtcccaggttatgacacacctccgtgtgatctacgagcgcatgaaccagtctctgtccctgctctacaatgtccctgcggtggctgaggagattcaagatgaagtcgatgagctgcttcagaaggagcagaactactccgacgatgtcttggccaacatgatcagtgagcccagaatcagctacggaaacgacgctctcatgccttcgctgacggaaaccaagaccaccgtggagctccttcccgtgaatggggaattcagcctggatgacctccagccgtggcacccttttggggtggactctgtgccagccaataccgaaaatgaagtcgagcctgttgacgcccgccccgctgctgaccgaggactgaccactcgaccaggttctgggctgacaaacatcaagacggaagagatctcggaagtgaagatggatgcagaattcggacatgattcaggatttgaagtccgccatcaaaaactggtgttctttgctgaagatgtgggttcgaacaaaggcgccatcatcggactcatggtgggcggcgttgtcatagcaaccgtgattgtcatcaccctggtgatgttgaagaagaaacagtacacatccatccatcatggcgtggtggaggtcgacgccgccgtgaccccagaggagcgccatctctccaagatgcagcagaacggatatgagaatccaacttacaagttctttgagcaaatgcagaactaa

SEQ ID NO: 8 - protein, 695 aa, Mus musculus
MLPSLALLLLAAWTVRALEVPTDGNAGLLAEPQIAMFCGKLNMHMNVQNGKWESDPSGTKTCIGTKEGILQYCQEVYPELQITNVVEANQPVTIQNWCKRGRKQCKTHTHIVIPYRCLVGEFVSDALLVPDKCKFLHQERMDVCETHLHWHTVAKETCSEKSTNLHDYGMLLPCGIDKFRGVEFVCCPLAEESDSVDSADAEEDDSDVWWGGADTDYADGGEDKVVEVAEEEEVADVEEEEADDDEDVEDGDEVEEEAEEPYEEATERTTSTATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPHHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHPFGVDSVPANTENEVEPVDARPAADRGLTTRPGSGLTNIKTEEISEVKMDAEFGHDSGFEVRHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 9 - DNA, 2313 bp, Rattus norvegicus
  CDS (1)..(2313)
atgctgcccagcctggcactgctcctgctggccgcctggacggttcgggctctggaggtgcccactgatggcaatgccggtctgctggcagaaccccagatcgccatgttctgtggtaaactcaacatgcacatgaatgtgcagaatggaaaatgggagtcagacccatcagggaccaaaacctgcattggcaccaaggagggaatcctgcagtactgccaagaggtctaccctgaactgcagatcacaaacgtggtggaagccaaccagccagtgaccatccagaactggtgcaagcggggccgcaagcagtgcaagacgcacacccacatcgtgattccttaccggtgcctagttggtgagtttgtaagcgatgcccttctcgtgcccgacaagtgcaagtttctacaccaggagcggatggacgtttgtgagacccatcttcactggcatactgttgccaaagagacatgcagtgagaagagcactaacttgcacgactatggcatgctgctgccctgtggcatcgacaagttccgaggggtcgagttcgtgtgctgcccgttggcggaggagagcgacagcatcgattctgcggacgcagaggaggacgactccgatgtctggtggggtggagcggacacagactatgctgatggcggtgaagacaaagtcgtagaagtagccgaagaggaggaagtggccgatgttgaggaagaagaagctgaggatgacgaggatgtggaggatggggatgaggtggaggaggaggccgaggagccctacgaagaggccacagagagaacaaccagcattgccaccactaccacaactaccactgagtctgtggaggaggtagtccgagaggtgtgctctgaacaagctgagaccgggccatgccgtgcaatgatctcccgctggtactttgatgtcactgaaggaaagtgcgccccattcttttacggcggatgtggcggcaacaggaacaactttgacactgaagagtactgcatggcggtgtgtggcagcgtgtcatcccaaagtttactcaagactaccagtgaacctcttccccaagatccggttaaacttcccacgacggcagccagcacccctgacgcagtcgacaagtacctggagacccccggagatgagaacgagcacgcccatttccagaaagccaaagagaggttggaagccaagcaccgagagagaatgtcccaggtcatgagagaatgggaggaggcagaacgtcaagccaagaatttgcccaaagctgacaagaaggccgttatccagcatttccaggagaaagtggaatctctggaacaggaagcagccaacgagaggcagcagcttgtagagacacacatggccagagttgaagccatgctcaatgatcgccgtcgcctggccctcgagaattacatcaccgcactgcaggcggtgcctccaaggcctcatcatgtgttcaacatgctgaagaagtacgtccgtgcagagcagaaggacagacagcacaccctaaagcattttgaacatgtgcgcatggtggaccccaagaaagctgctcagatccggtcccaggttatgacacacctccgtgtgatctacgagcgcatgaaccagtctctgtccctgctctacaacgtccctgccgtggctgaggagattcaagatgaagttgacgagctgcttcagaaggagcagaactactccgacgacgtcttagccaacatgatcagtgaacccagaatcagttacggcaacgatgctctcatgccttctttgactgaaacgaagaccactgtggagctccttcccgtgaatggcgaattcagcctggatgatctccaaccgtggcatccttttggggtggactctgtgccagccaatacagaaaatgaagttgagcctgtcgacgcccgccccgctgctgaccgaggactgaccactcgaccagggtctgggttgacaaacatcaagacagaagagatctcagaagtgaagatggatgcggagttcggacatgattcaggcttcgaagtccgccatcaaaaactggtgttctttgcagaagatgtgggttcaaacaaaggtgccatcattggactcatggtgggtggcgttgtcatagcaacagtgattgtcatcaccttggtgatgctgaagaagaaacagtacacatccatccatcatggcgtggtggaggttgacgctgctgtgaccccggaggagcgccacctctccaagatgcagcagaatggatatgagaatccaacatacaagttctttgagcagatgcagaactaa

SEQ ID NO: 10 - protein, 770 aa, Rattus norvegicus
MLPSLALLLLAAWTVRALEVPTDGNAGLLAEPQIAMFCGKLNMHMNVQNGKWESDPSGTKTCIGTKEGILQYCQEVYPELQITNVVEANQPVTIQNWCKRGRKQCKTHTHIVIPYRCLVGEFVSDALLVPDKCKFLHQERMDVCETHLHWHTVAKETCSEKSTNLHDYGMLLPCGIDKFRGVEFVCCPLAEESDSIDSADAEEDDSDVWWGGADTDYADGGEDKVVEVAEEEEVADVEEEEAEDDEDVEDGDEVEEEAEEPYEEATERTTSIATTTTTTTESVEEVVREVCSEQAETGPCRAMISRWYFDVTEGKCAPFFYGGCGGNRNNFDTEEYCMAVCGSVSSQSLLKTTSEPLPQDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPHHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHPFGVDSVPANTENEVEPVDARPAADRGLTTRPGSGLTNIKTEEISEVKMDAEFGHDSGFEVRHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 11 - DNA, 111 bp, Homo sapiens
  CDS (1)..(111)
gatgcagaattccgacatgactcaggatatgaagttcatcatcaaaaattggtgttctttgcagaagatgtgggttcaaacaaaggtgcaatcattggactcatggtgggc

SEQ ID NO: 12 - protein, 37 aa, Homo sapiens
DAEFRHDSGYEVHHQKLVFFAEDVGSNKGAIIGLMVG

SEQ ID NO: 13 - DNA, 111 bp, Mus musculus
  CDS (1)..(111)
gatgcagaattcggacatgattcaggatttgaagtccgccatcaaaaactggtgttctttgctgaagatgtgggttcgaacaaaggcgccatcatcggactcatggtgggc

SEQ ID NO: 14 - protein, 37 aa, Mus musculus
DAEFGHDSGFEVRHQKLVFFAEDVGSNKGAIIGLMVG

SEQ ID NO: 15 - DNA, 111 bp, Rattus norvegicus
  CDS (1)..(111)
gatgcggagttcggacatgattcaggcttcgaagtccgccatcaaaaactggtgttctttgcagaagatgtgggttcaaacaaaggtgccatcattggactcatggtgggt

SEQ ID NO: 16 - protein, 37 aa, Rattus norvegicus; identical to SEQ ID NO: 14

SEQ ID NO: 17 - DNA, 114 bp, Homo sapiens
  CDS (1)..(114)
gatgcagaattccgacatgactcaggatatgaagttcatcatcaaaaattggtgttctttgcagaagatgtgggttcaaacaaaggtgcaatcattggactcatggtgggcggt

SEQ ID NO: 18 - protein, 38 aa, Homo sapiens
DAEFRHDSGYEVHHQKLVFFAEDVGSNKGAIIGLMVGG

SEQ ID NO: 19 - DNA, 114 bp, Mus musculus
  CDS (1)..(114)
gatgcagaattcggacatgattcaggatttgaagtccgccatcaaaaactggtgttctttgctgaagatgtgggttcgaacaaaggcgccatcatcggactcatggtgggcggc

SEQ ID NO: 20 - protein, 38 aa, Mus musculus
DAEFGHDSGFEVRHQKLVFFAEDVGSNKGAIIGLMVGG

SEQ ID NO: 21 - DNA, 114 bp, Rattus norvegicus
  CDS (1)..(114)
gatgcggagttcggacatgattcaggcttcgaagtccgccatcaaaaactggtgttctttgcagaagatgtgggttcaaacaaaggtgccatcattggactcatggtgggtggc

SEQ ID NO: 22 - protein, 38 aa, Rattus norvegicus; identical to SEQ ID NO: 20